(19) United States
(12) Patent Application Publication
(10) Pub. No.: US 2024/0279466 A1
(43) Pub. Date: Aug. 22, 2024

(54) AN AMINOSQUARAMIDE POLYMER

(71) Applicants: UNIVERSITAT DE LES ILLES BALEARS, PALMA DE MALLORCA (ES); CHARIOT INNOVATIONS LIMITED, LONDON (GB)

(72) Inventors: MARIA DEL CARMEN ROTGER PONS, PALMA (ES); ANTONIO COSTA TORRES, PALMA (ES); PRIAM DE VILLALONGA SMITH, PALMA (ES); SILVIA FERNÁNDEZ DE MATTOS; FRANCISCA ORVAY PINTOS, PALMA (ES); FRANCISCO OLMO ARÉVALO, LONDON (GB); JOHN MORRISON KELLY, HERTFORDSHIRE (GB); BARTOMEU GALMÉS SUREDA, PALMA (ES)

(21) Appl. No.: 18/579,047

(22) PCT Filed: Jul. 14, 2022
(86) PCT No.: PCT/EP2022/069806

§ 371 Date: Jan. 12, 2024

(30) Foreign Application Priority Data
Jul. 15, 2021 (EP) 21382639.9

(51) Int. Cl. C08L 79/02; A61K 47/34; C08G 73/02

(52) U.S. Cl. CPC C08L 79/02; A61K 47/34; C08G 73/0206; C08L 2203/02

(57) ABSTRACT

The present invention relates to an aminosquaramide polymer comprising a backbone of repeating structural units, being these repeating structural units equal or different and selected from the group consisting of: monomer of formula (A) (or a salt thereof), and monomer of formula (B) (or a salt thereof); and a conjugate comprising the aminosquaramide polymer and one or more molecules of interest. It also refers to compositions containing the polymer or the conjugate; and processes for their preparation, as well as their uses as a carrier, in therapy, in diagnostic, in cosmetic and as a transfecting agent.

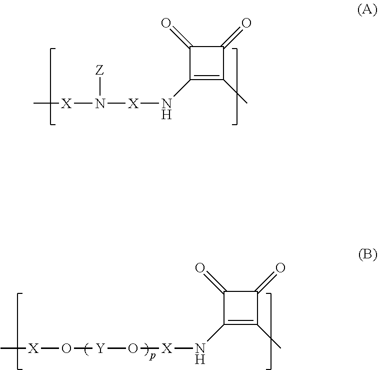

This application claims the benefit of European Patent Application EP21382639.9 filed on Jul. 15, 2021.

This invention relates to the field of polymers. In particular to an aminosquaramide polymer, a conjugate containing it, compositions containing them and their preparation processes. The present invention also relates to their use as a carrier and also in therapy, cosmetic, diagnostic and as a transfecting agent.

### BACKGROUND ART

The delivery of (exogenous) nucleotides into cells is essential for cell and molecular biology research and biotechnology, being a promising approach for the treatment of diseases related to the malfunction of defective genes. The delivery of nucleotides such as DNA, RNA or oligonucleotides into cells is called transfection/transformation and enables the study of gene function and gene products in cells. Depending on both, the nature of the genetic materials (DNAs and RNAs) introduced into the cell and the persistence of the genetic material, transfection can be stable or transient. Stable transfection introduces genetic materials (DNAs) that are integrated into the host genome and sustain transgene expression even after host cell replication. In contrast, transient transfection introduces genetic materials (RNAs) that are only expressed for a limited period of time or DNAs that are not integrated into the genome but can be maintained by selective drug markers.

Therefore, transfection is commonly used for a plethora of applications such as studies on gene function or expression of proteins of interest as well as in RNA interference experiments. Transfection can also be applied to in vivo experiments to validate in vitro gene silencing or gene overexpression experiments. Transfection is also used in applications and methods of high-throughput cell-based screening, and can also be adapted to bioproduction at small or large scale of gene delivery, virus, and protein production, including antibody production through DNA vaccines. The need for the development of targeted therapies has revealed human gene therapy as a promising approach of treatment.

Gene therapy aims to treat a disease by transferring one or more therapeutic nucleic acids to a patient's cells, or by correcting defective genes through gene editing mechanisms or small interfering RNA (siRNA) to specifically inhibit the expression of complementary RNA transcripts. Therefore, gene delivery relies on methodologies that facilitate the internalization of nucleic acids to reach the appropriate intracellular place at the appropriate time. Depending on the host characteristics, the delivery of nucleic acids is a non-straightforward task. For these reasons, many different techniques have been developed and disclosed in the state of the art.

Gene delivery methods can be categorized as biological, physical, or chemical approaches. Firstly, biological methods include the use of recombinant viruses (i.e. adenovirus, adeno associated virus, and lentivirus) and conjugation. Delivery of foreign DNA using viral vectors is effective but must deal with several problems such as the small size of DNA that can be transported and its immunogenicity, which can cause fatal adverse reactions, abrogate their activity, or require additional immunosuppressive therapy. Additionally, there are limitations in large-scale production of viruses and their potential to induce undesired insertional mutagenesis strongly reduces their suitability for gene therapy. A few modified viral vectors have been approved for the treatment of human disease, such as Glybera for lipoprotein lipase deficiency, Gendicine for cancer and Luxturna for retinal dystrophy. However, Glybera was withdrawn from the market due to the high production costs and the lack of demand.

Secondly, there are several physical methods that can be used for gene delivery into cells. Physical approaches include microinjection, optical transfection, particle guns (ballistic gene delivery), electroporation, sonoporation, magnetofection, and electric field induced molecular vibration. Some methods have been established for cell lines considered hard-to-transfect, both in suspension and adherent, for primary isolated cells, and to some extent in tissue. Due to their intrinsic characteristics physical transfection methods are useful for ex vivo clinical applications such as chimeric antigen receptor (CAR) T cell therapy. However, the in vivo applications of physical transfection methods are limited to cutaneous treatments or local hydrodynamic gene delivery to liver or muscle. In general, physical transfection methods have many drawbacks since they are technologically demanding methodologies, with some of them like μ-tool based thermoporation only found in specialized labs. Furthermore, they facilitate transfection of a limited number of cells and some, such as electroporation require, specific adaptations and optimization of the parameters for particular cell types and tends to generate high levels of damaged cells.

Thirdly, as an alternative to physical transfection methods and biological methods, the use of chemical vectors for gene delivery has become a promising approach that tries to overcome the several critical issues that are bound to physical methods and their inability for in vivo use. Chemical vectors are safer than viral vectors, cheaper, more reproducible and do not present DNA size limit. However, the main limitation of non-viral systems is their low transfection efficiency, although this has been improved by different strategies and the efforts are still ongoing; actually, advances in non-viral delivery have led to an increased number of products entering into clinical trials. Unfortunately, the transfection efficiency with standard non-viral vectors is low due to low cell selectivity and endosomal entrapment. The fact that most of the gene delivery studies have been carried out in monolayer cultures of immortalized cells lines have overestimated the in vivo efficiency of gene delivery methods.

Nonetheless, important advances in the concept and design of chemical vectors have been made in the past few years. As examples, cationic lipid based non-viral vectors, polymeric-based non-viral vectors and cell penetrating peptides (CPPs) have been disclosed in the state of the art.

Firstly, lipofection has been one of the most popular methods of transfection since the discovery that cationic lipids condense DNA and fuse with cell membranes. Many efforts have been made to enhance the fusogenic properties of the liposomes and minimize the toxicity. Lipid nanoparticles (LNPs) of approximately 100 nm, when mixed with polyanionic oligonucleotides, represent the most advanced delivery platforms for systemic administration of siRNA therapeutics. However, studies on structure-activity relationship of cationic lipids have shown that permanently charged lipids bearing quaternary amine groups in the hydrophilic head region were less effective in in vivo gene knockdown experiments. For this reason, complex mixtures have been developed by adding fusogenic peptides into the lipid particles to enhance their cell internalization properties or by incorporating into the lipid particles formulation the transfection helper lipid dioleoyl phosphatidylethanolamine (DOPE), whose conic shape tends to assemble into the less stable hexagonal phase rather than the lamellar phase adopted by other lipids. These types of particles need additional stabilization, compromising their future applications in vivo.

Secondly, polymeric-based non-viral vectors has been also disclosed in the state of the art. In particular, artificial “self-delivering” oligonucleotides are oligomers of chemically-modified nucleotides that improve stability and uptake efficiency while retaining their target recognition ability. This sort of nucleotides includes antisense phosphonothioates, and the inclusion of additional hydrophobic carbon atoms between the C5′ and C3′ positions of the nucleotide. However, nucleotide chemical modifications have adverse effects such as complement activation, thrombocytopenia, increased off-target interactions or the intrinsic toxicity of metabolites. Alternatively, esterification and phosphotriester formation reduces the highly negative charged phosphate backbone responsible for blocking their passage through the nonpolar membrane.

Thirdly, cell penetrating peptides (CPPs) are positively charged short peptides (5-30 aa long) with high internalization efficiency and low to moderate cytotoxicity that form covalent or non-covalent conjugates. CPPs have been used for nucleic acids and other cargoes delivery into cells. Amphiphilic and arginine rich-CPPs internalize well into cells due their high electrostatic interaction with negatively charged cellular membranes, but efficiency depends on parameters such size and complexity of the cargo-CPP, nature of CPP, and the type of peptide sequence. Extensive research has recently focused on the development of synthetic CPPs. However, covalent conjugation to the CPPs requires time consuming processes and tailor-made linkers and conjugation, depending on the cargo, while non-covalent conjugates rely mainly on electrostatic interactions to bind the oligonucleotide or cargo to the peptide. Despite their advantages, such as high transduction efficiency and low cytotoxicity, CPPs have some limitations such as the lack of cell specificity, and most importantly, their uptake into intracellular endosomes from which they must be released using auxiliary compounds such as charged polymers that may be toxic to other organelles. Additionally, CPPs are inactivated by serum proteases. To prevent this, CPPs are stabilized by the incorporation of non-proteinogenic amino acids or cyclization to avoid protease-mediated degradation.

As it is disclosed in the state of the art, polymers have been widely used as carriers in a large number of applications, including biomedical applications (molecular biology and gene therapy). These polymers require a suitable solubility in the physiological media and an appropriate degradation profile to retain their stability during targeting, and to allow release of the appropriate amount of the molecule of interest in the target sites.

As it is disclosed above, polymers has been used to improve methodologies for genetic modification, through increased transfection efficiency, enhanced genome editing, controllable expression, DNA repair, and gene activation, inhibition, or regulation. Thus, polymers are structurally versatile compounds that can be obtained from units that contain the functionalities required for nucleic acid interaction, membrane recognition and translocation. This, together with their potential synthetic scalability, converts polymers into one of the best non-natural materials for gene delivery. Recent efforts into the development of polymers for gene delivery are focused on improving nucleic acid condensation capability, reducing toxicity, and enhancing endosomal escape. To achieve these goals, the new polymers are designed to disassemble in response to external stimuli such as pH, reducing agents, temperature, and enzymes.

In particular, in the United States patent application number US2011294772, the use of oligo-squaramide-based cyclic compounds containing up to 18 squaramide equal structural repeating units are disclosed as inhibiting agent of a series of kinases and as anti-tumorous agents. However, it is not disclosed squaramide-based cyclic compounds comprising different monomers having different backbone structure; and neither the use as a carrier of squaramide-based cyclic compounds nor conjugates or compositions comprising squaramide-based polymers.

Further, it is also known in the state of the art macrocyclization of pre-organized palindromic oligo-squaramide having a maximum of six repeating units (cf. Rotger et al. “Efficient macrocyclization preorganized palindromic oliosquaramides”, Angewandte Chemie International Edition, Verlag Chemie, 2006, vol. 45 (41), pp. 6844-6848). And Chasák et al. discloses oligo-aminosquaramide cyclic compounds having up to seven repeating units having equal backbone. However, none of them disclose aminosquaramide polymers having 10 or more repeating units, neither a conjugate comprising them (cf. Squaric acid analogues in medicinal chemistry”, European journal of Medicinal Chemistry. 2020, vol. 209).

Unfortunately, despite these efforts, the major drawback that still hinders gene delivery based on chemical-mediated methods, and particularly with the use of polymers, is their variable efficacy and relatively high toxicity. Thus, there is still the need to develop a stable and effective polymer-based carrier/vector systems that have low toxicity; particularly for use in gene therapy.

### SUMMARY OF INVENTION

The inventors have surprisingly found that a polymer based on aminosquaramide repeating units is useful as a carrier; and particularly useful as an artificial chemical-mediated vector for the delivery of molecules of interest, such as nucleotides for gene delivery; or alternatively as an active ingredient delivery system. As is demonstrated in the experimental section, the aminosquaramide-based polymer of the present invention, are suited to delivery (biologically active) molecules into a wide variety of cells, such as cell lines commonly used in research laboratory settings, cultivable protozoan parasites, free-living protists, bacteria, and in addition, cells lines and lineages that are typically considered difficult to transfect. In fact, molecules of interest retain activity after delivery.

In fact, the aminosquaramide-based polymer of the present invention is a versatile carrier for the delivery of any biologically active molecule of interest through the rapid formation of a conjugate between the polymer and the molecule of interest by non-covalent bonds. Examples of molecules of interest include, but is not limited to, active ingredients (such as pharmaceutically, veterinary and cosmetic active ingredients); amino-acid containing compounds (such as polypeptides and proteins; including antibodies and fragments thereof); and nucleic acid-containing compounds (such as oligonucleotides and modified versions thereof, including single strand oligonucleotides DNA, RNA (siRNA, miRNA, sgRNA, PNA, LNA and their analogues), double-strand oligonucleotides (siRNA, shRNA, decoy DNA), oDNA, plasmids and other varieties thereof.

In fact, the use of the aminosquaramide-based polymer of the present invention allows the simultaneous delivery of several molecules of interest conjugated to the polymer. In particular, the polymer of the invention allows transfection of up to about 10 nucleic acid expression sequences that encode distinct polypeptides. In addition, non-coding RNA molecules such as siRNA, miRNA, or single guide RNA (sgRNA) molecules used in genome editing experiments can also be introduced. Further, with CRISPR-mediated genome editing, sgRNA can be transfected in combination with the relevant DNA molecules.

In addition, the backbone of the aminosquaramide-based polymer of the invention can be further modified by including in the structural repeating units one or more active ingredients (such as pharmaceutically, veterinary, and cosmetic active ingredients), and/or detection agents (such as chromophore moiety, fluorescent moiety, phosphorescent moiety, luminescent moiety, light absorbing moiety, radioactive moiety, transition metal and isotope mass tag moiety). Therefore, the aminosquaramide-based polymer can be used in lab research, therapy, cosmetic or diagnostic circumstances.

In fact, the use of the aminosquaramide-based polymer of the present invention allows the simultaneous delivery of several molecules of interest conjugated to the polymer by non-covalent bonds, in combination with active ingredients or detection agents bonded to the same structure of the polymer.

The aminosquaramide polymer of the present invention is thermally and chemically stable under the manufacturing, storage and use conditions. In particular, the polymer of the invention is also easily handled, adaptable and cost effective. Further, the polymer of the present invention is obtained by a simple and non-expensive polycondensation reaction under mild conditions that are easily scalable to an industrial level, and the delivery/transfection process involves a simple protocol that does not require any special/costly material or equipment.

Thus, the aminosquaramide polymer of the present invention is effective for use as a carrier/vector for the delivery of molecules of interest to the target site; particularly for the transfection of nucleic acid containing compounds into eukaryote cells. As demonstrated in the experimental section, the aminosquaramide polymer of the present invention is capable of successfully transfecting a wide range of mammalian cell lines, including non-transformed murine fibroblasts (NIH3T3) and human embryonic kidney cells (HEK293), together with human xeroderma pigmentosum cells (XP2YO)) and human cancer cell lines (PC-3, metastatic prostate cancer; SW480, colorectal cancer). In addition, it can be applied to many types of unicellular organisms, and to multicellular organisms.

Therefore, the first aspect of the invention refers to an aminosquaramide polymer comprising a backbone of repeating structural units, being these repeating structural units equal or different and selected from the group consisting of:
a monomer of formula A, or a salt thereof

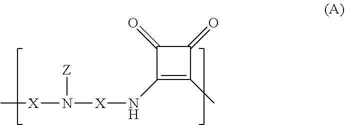

and a monomer of formula B, or a salt thereof

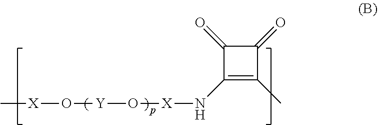

or a salt thereof,
wherein:
X is selected from the group consisting of —(CH₂)_{q}—, —(CHR₁)_{q}— and —(CR₁R₂)_{q}—;
Y is selected from the group consisting of —(CH₂)ᵣ—, —(CHR₃)ᵣ— and —(CR₃R₄)ᵣ—;
R₁ and R₂ are independently selected from the group consisting of H and Z′;
R₃ and R₄ are independently selected from the group consisting of H and Z′;
each Z and Z′ are independently selected from the group consisting of an inert moiety, a detection moiety, and an active moiety;
the inert moiety is selected from the group consisting of —(C₁-C₁₂)alkyl; —(C₂-C₁₂)alkenyl; —(C₂-C₁₂)alkylene-O—(Y—O)_{w}—(C₁-C₁₂)alquil, —(C₂-C₁₂)alkylene-O—(Y—O)_{w}—(C₂-C₁₂)alkylene-NR₁₁R₁₃, —(C₂-C₆)alkynyl; -Cy1; —(C₁-C₁₂)alkylene-Cy1; —(C₂-C₁₂)alkenylene-Cy1; —(C₂-C₁₂)alkynylene-Cy1; -Cy2-(C₁-C₁₂)alkyl; -Cy2-(C₂-C₁₂)alkenyl; and -Cy2-(C₂-C₁₂)alkynyl; being Cy1 a known ring system independently selected from the group consisting of 5- to 7-membered saturated or partially unsaturated carbocyclic or heterocyclic ring; and 5- to 7-membered carbocyclic or heterocyclic aromatic ring; Cy2 a bivalent known ring system independently selected from the group consisting of 5- to 7-membered saturated or partially unsaturated carbocyclic or heterocyclic ring; and 5- to 7-membered carbocyclic or heterocyclic aromatic ring; and being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl; being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl;
R₅ and R₆ are independently selected from the group consisting of —(C₁-C₁₂)alkyl and an amine protecting group;
R₁₁ and R₁₃ are independently selected from the group consisting of H, —(C₁-C₁₂)alkyl and an amine protecting group;
Halogen is selected from the group consisting of F, Cl, Br, and I;
the detection moiety is selected from the group consisting of chromophore moiety, fluorescent moiety, phosphorescent moiety, luminescent moiety, light absorbing moiety, radioactive moiety, transition metal and isotope mass tag moiety; and
the active moiety is selected from the group consisting of a pharmaceutically active ingredient, a veterinary active ingredient, and a cosmetic active ingredient;
p is an integer from 1 to 10;
q is an integer from 2 to 6;
r is an integer from 2 to 4;
the percentage by weight of the monomer of formula A or a salt thereof is from 30% to 100%;
the percentage by weight of the monomer of formula B or a salt thereof is from 0% to 70%;
being the sum of the percentages equal to 100%; and
the aminosquaramide polymer has a weight average molecular weight (Mw) from 2000 to 91000 Da; with the proviso that when the percentage by weight of monomer A is 100%, then the weight average molecular weight (Mw)) of the polymer is from 4500 to 91000 Da;
being:
the weight average molecular weight (Mw) from 2000 to 30000 Da measured by Gel Permeation Chromatography-Visible Ultraviolet (GPC-UV); and
the weight average molecular weight (Mw) from higher than 30000 to 91000 Da measured by analytical size-exclusion chromatography coupled with multi-angle light scattering and refractive index detector (SEC/MALS/RI).

The second aspect of the invention refers to a conjugate comprising a aminosquaramide polymer comprising a backbone of repeating structural units, being these repeating structural units equal or different and selected from the group consisting of:
A monomer of formula A, or a salt thereof

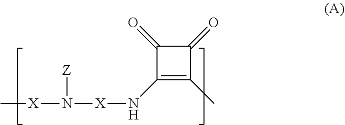

and a monomer of formula B, or a salt thereof

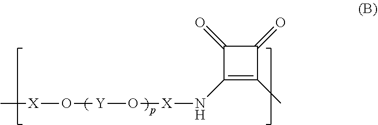

wherein:
X is selected from the group consisting of —(CH₂)_{q}—, —(CHR₁)_{q}— and —(CR₁R₂)_{q}—;
Y is selected from the group consisting of —(CH₂)ᵣ—, —(CHR₃)ᵣ— and —(CR₃R₄)ᵣ—;
R₁ and R₂ are independently selected from the group consisting of H and Z′;
R₃ and R₄ are independently selected from the group consisting of H and Z′;
each Z and Z′ are independently selected from the group consisting of an inert moiety, a detection moiety, and an active moiety;
the inert moiety is selected from the group consisting of —(C₁-C₁₂)alkyl; —(C₂-C₁₂)alkenyl; —(C₂-C₁₂)alkylene-O—(Y—O)_{w}—(C₁-C₁₂)alquil, —(C₂-C₁₂)alkylene-O—(Y—O)—(C₂-C₁₂)alkylene-NR₁₁R₁₃, —(C₂-C₆)alkynyl; -Cy1; —(C₁-C₁₂)alkylene-Cy1; —(C₂-C₁₂)alkenylene-Cy1; —(C₂-C₁₂)alkynylene-Cy1; -Cy2-(C₁-C₁₂)alkyl; -Cy2-(C₂-C₁₂)alkenyl; and -Cy2-(C₂-C₁₂)alkynyl; being Cy1 a known ring system independently selected from the group consisting of 5- to 7-membered saturated or partially unsaturated carbocyclic or heterocyclic ring; and 5- to 7-membered carbocyclic or heterocyclic aromatic ring; Cy2 a bivalent known ring system independently selected from the group consisting of 5- to 7-membered saturated or partially unsaturated carbocyclic or heterocyclic ring; and
5- to 7-membered carbocyclic or heterocyclic aromatic ring; and being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl; being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl;
R₅ and R₆ are independently selected from the group consisting of —(C₁-C₁₂)alkyl and an amine protecting group;
R₁₁ and R₁₃ are independently selected from the group consisting of H, —(C₁-C₁₂)alkyl and an amine protecting group;
Halogen is selected from the group consisting of F, Cl, Br, and I;
the detection moiety is selected from the group consisting of chromophore moiety, fluorescent moiety, phosphorescent moiety, luminescent moiety, light absorbing moiety, radioactive moiety, transition metal and isotope mass tag moiety; and
the active moiety is selected from the group consisting of a pharmaceutically active ingredient, a veterinary active ingredient, and a cosmetic active ingredient;
p is an integer from 1 to 10;
q is an integer from 2 to 6;
r is an integer from 2 to 4;
the percentage by weight of the monomer of formula A, or a salt thereof is from 30% to 100% by weight;
the percentage by weight of the monomer of formula B, or a salt thereof is from 0% to 70% by weight; being the sum of the percentages equal to 100%; and
the aminosquaramide polymer has a weight average molecular weight (Mw) from 2000 to 91000 Da being:
being:
the weight average molecular weight (Mw) from 2000 to 30000 Da measured by Gel Permeation Chromatography-Visible Ultraviolet (GPC-UV); and
the weight average molecular weight (Mw) from higher than 30000 to 91000 Da measured by analytical size-exclusion chromatography coupled with multi-angle light scattering and refractive index detector (SEC/MALS/RI);
and one or more molecules of interest.

The third aspect of the invention refers to a composition comprising the polymer as defined in the second aspect of the invention; or alternatively the conjugate of the second aspect of the invention, together with one or more appropriate excipients or carriers.

The fourth aspect of the invention refers to the use of the polymer as defined in the second aspect of the invention, or the composition of the third aspect of the invention, as a carrier, wherein Z and Z′ are inert moieties.

The fifth aspect of the invention refers to a polymer as defined in the second aspect of the invention, a conjugate of the second aspect of the invention; or a composition of the third aspect of the invention for use in therapy wherein the polymer comprises at least one or more therapeutically or veterinary active moieties and/or the conjugate comprises at least a molecule of interest selected from the group consisting of a pharmaceutically or veterinary active ingredient, an amino acid-containing compound, a nucleic acid-containing compound, or a mixture thereof.

The sixth aspect of the invention refers to a polymer as defined in the second aspect of the invention, a conjugate of the second aspect of the invention; or a composition of the third aspect of the invention for use in diagnostic wherein the polymer comprises at least one detection moieties.

The seventh aspect of the invention refers to a conjugate of the second aspect of the invention; or a composition of the third aspect of the invention for use as a transfecting agent, wherein the molecule of interest is at least a nucleic acid-containing compound.

The eighth aspect of the invention refers to the use of a polymer as defined in the second aspect of the invention, a conjugate of the second aspect of the invention; or a composition of the third aspect of the invention in cosmetics, wherein the polymer comprises at least a cosmetically active moiety and/or the conjugate wherein the molecule of interest is at least one cosmetically active ingredient.

It is also an aspect of the invention a kit comprising the polymer as defined in the second aspect of the invention, or a conjugate of the second aspect of the invention; or a composition of the third aspect of the invention; and means for their use.

### BRIEF DESCRIPTION OF DRAWINGS

FIG. 1. Section A corresponds to the ¹HNMR spectrum (recorded in H₂O containing 0.1% v/v HCOOH) of the high molecular weight fraction of mixture 1 of the present invention obtained by method B showing the four relatively broad peaks assigned to the four distinct proton types present in the aliphatic portion of the compounds forming part of mixture 1. (i.e. about 2.07 ppm, about 2.90 ppm, about 3.22 ppm and about 3.64 ppm); and the structural features of the inner aminosquaramide units and a diagnostic triplet at about 3.08 ppm (enlarged area), assigned to the terminal protonated amino methylene groups.

Section B corresponds to the ¹³C NMR spectrum (recorded in H₂O containing 0.1% v/v HCOOH using DSS as an internal reference) of the high molecular weight fraction of mixture 1 obtained by method B of the present invention showing the corresponding carbon resonances of the aliphatic portion of the compounds forming part of mixture 1 (i.e. about 28.23 ppm, about 42.91 ppm, about 44.10 ppm and about 56.22 ppm); and the structural features of the inner aminosquaramide units and a diagnostic peak at about 39.39 ppm assigned to the terminal amino methylene groups.

FIG. 2(a) shows the Green fluorescent protein (GFP) expression relative to Lipofectamine 2000 in comparison with the aminosquaramide treated cells (C+) and (b) shows cell viability relative to untreated cells in comparison with the aminosquaramide treated cells (C−). Grid bars correspond to Lipofectamine treated cells and solid black bars to aminosquaramide treated cells.

FIG. 3 shows the Green fluorescent protein (GFP) expression achieved by electroporation (physical transfection method) (dotted bars), and with the aminosquaramide treated cells (solid bars).

FIG. 4 shows the epi-fluorescence microscopy images after transfection of the plasmid codifying DsRed protein with mixture 3 of the present invention, which comprises bodipy as detection moieties in 25% of the repeating structural units of the polymer. The green areas (viewed with the green light channel) and marked with the letter (a) show the presence of the aminosquaramide polymer mixture 3 of the present invention. And the red areas (viewed with the red light channel) and marked with the letter (b) show the presence of the red fluorescent protein (i.e. DsRed protein) synthetized as a result of transfection of the plasmid encoding the DsRed protein with the squaramide polymer mixture 3 of the present invention.

### DETAILED DESCRIPTION OF THE INVENTION

As it is mentioned above, the first aspect of the invention refers to an aminosquaramide polymer comprising a backbone of repeating structural units equal or different of monomer of formula (A), or a salt thereof; or alternatively, the polymer comprises a backbone of repeating structural units equal or different of at least 30% of monomer of formula (A), or a salt thereof; and up to 100% of monomer of formula (B), or a salt thereof.

In an embodiment, the aminosquaramide polymer is one wherein the repeating structural units are equal or different of formula (II) or a salt thereof,

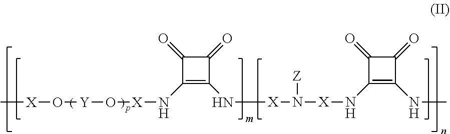

wherein:
m is 0 or 1;
with the proviso that;
when m is 0, then the % by weight of the monomer of formula A, or a salt thereof is 100% and n is an integer from 20 to 400; and
when m is 1, then the percentage of formula A or a salt thereof is 50%, and the percentage of formula B or a salt thereof is 50% and n is an integer from 10 to 400.

In an embodiment, the aminosquaramide polymer is one wherein the repeating structural units are equal or different of formula (II) or a salt thereof,

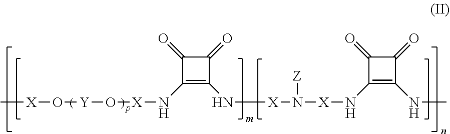

wherein:
X is selected from the group consisting of —(CH₂)_{q}—, —(CHR₁)_{q}— and —(CR₁R₂)_{q}—;
Y is selected from the group consisting of —(CH₂)ᵣ—, —(CHR₃)ᵣ— and —(CR₃R₄)ᵣ—;
R₁ and R₂ are independently selected from the group consisting of H and Z′;
R₃ and R₄ are independently selected from the group consisting of H and Z′;
each Z and Z′ are independently selected from the group consisting of an inert moiety, a detection moiety, and an active moiety;
the inert moiety is selected from the group consisting of —(C₁-C₁₂)alkyl; —(C₂-C₁₂)alkenyl; —(C₂-C₆)alkynyl; -Cy1; —(C₁-C₁₂)alkylene-Cy1; —(C₂-C₁₂)alkenylene-Cy1; —C₂-C₁₂)alkynylene-Cy1; -Cy2-(C₁-C₁₂)alkyl; -Cy2-(C₂-C₁₂)alkenyl; and -Cy2-(C₂-C₁₂)alkynyl; being Cy1 a known ring system independently selected from the group consisting of 5- to 7-membered saturated or partially unsaturated carbocyclic or heterocyclic ring; and 5- to 7-membered carbocyclic or heterocyclic aromatic ring; Cy2 a bivalent known ring system independently selected from the group consisting of 5- to 7-membered saturated or partially unsaturated carbocyclic or heterocyclic ring; and 5- to 7-membered carbocyclic or heterocyclic aromatic ring; and being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl; being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl;
R₅ and R₆ are independently selected from the group consisting of —(C₁-C₁₂)alkyl and an amine protecting group;
Halogen is selected from the group consisting of F, Cl, Br, and I;
the detection moiety is selected from the group consisting of chromophore moiety, fluorescent moiety, phosphorescent moiety, luminescent moiety, light absorbing moiety, radioactive moiety, transition metal and isotope mass tag moiety; and
the active moiety is selected from the group consisting of a pharmaceutically active ingredient, a veterinary active ingredient, and a cosmetic active ingredient;
n is an integer from 20 to 180;
m is 0 or 1;
p is an integer from 1 to 10;
q is an integer from 2 to 6; and
r is an integer from 2 to 4.

Therefore, the polymer comprises a backbone of repeating structural units, being these repeating structural units equal or different of formula (II) or a salt thereof.

The salts of the repeating units of monomer (A) and monomer (B); and particularly of formula (II), encompasses salts formed from acceptable non-toxic acids including inorganic or organic acids. There is no limitation regarding the salts, except that they must be therapeutically (pharmaceutically or veterinary), diagnostic or cosmetic acceptable when they are used for therapeutic (pharmaceutical or veterinary), diagnostic or cosmetic purposes, respectively. Most of the acceptable salts are commercially available. If not, these salts can be prepared following the processes disclosed in the state of the art, which involves starting from acceptable non-toxic acids, including inorganic and organic acids. Such acids include for instance acetic, benzenesulfonic, benzoic, camphor sulfonic, citric, ethanesulfonic, fumaric, gluconic, glutamic, hydrobromic, hydrochloric, lactic, maleic, malic, mandelic, methanesulfonic, phosphoric, succinic, sulfuric, tartaric, p-toluenesulfonic acid, and formic acid. For instance, they can be prepared from the parent compound, which contains a basic or acidic moiety, by conventional chemical methods. Generally, such salts are, for example, prepared by reacting the free acid or base forms of these compounds with a stoichiometric amount of the appropriate acceptable base or acid in water or in an organic solvent or in a mixture of them.

For the purpose of the present invention, the term “pharmaceutically acceptable salts” used herein encompasses any salt formed from pharmaceutically acceptable non-toxic acids as defined above. The term “veterinary acceptable salts” used herein encompasses any salt formed from veterinary acceptable non-toxic acids as defined above. The term “diagnostic acceptable salts” used herein encompasses any salt formed from diagnostic acceptable non-toxic acids as defined above. The term “cosmetic acceptable salts” used herein encompasses any salt formed from cosmetic acceptable non-toxic acids as defined above.

For the purpose of the invention, the term “moiety” refers to a specific segment or functional group of a molecule or compound.

In particular, the moieties Z and Z′ can be selected from an inert moiety, a detection moiety, and an active moiety. The term “inert moiety” refers to a moiety that does not interfere substantially with the physico-chemical characteristics of the polymer and is selected from the group consisting of: —(C₁-C₁₂)alkyl; —(C₂-C₁₂)alkenyl; —(C₂-C₁₂)alkylene-O—(Y—O)—(C₁-C₁₂)alquil, —(C₂-C₁₂)alkylene-O—(Y—O)—(C₂-C₁₂)alkylene-NR₁₁R₁₃, —(C₂-C₆)alkynyl; -Cy1; —(C₁-C₁₂)alkylene-Cy1; —(C₂-C₁₂)alkenylene-Cy1; —(C₂-C₁₂)alkynylene-Cy1; -Cy2-(C₁-C₁₂)alkyl; -Cy2-(C₂-C₁₂)alkenyl; and -Cy2-(C₂-C₁₂)alkynyl; being Cy1 a known ring system independently selected from the group consisting of 5- to 7-membered saturated or partially unsaturated carbocyclic or heterocyclic ring; and 5- to 7-membered carbocyclic or heterocyclic aromatic ring; Cy2 a bivalent known ring system independently selected from the group consisting of 5- to 7-membered saturated or partially unsaturated carbocyclic or heterocyclic ring; and 5- to 7-membered carbocyclic or heterocyclic aromatic ring;
and being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl. Particularly selected from the group consisting of: —(C₁-C₁₂)alkyl; —(C₂-C₁₂)alkenyl; —(C₂-C₆)alkynyl; -Cy1; —(C₁-C₁₂)alkylene-Cy1; —(C₂-C₁₂)alkenylene-Cy1; —(C₂-C₁₂)alkynylene-Cy1; -Cy2-(C₁-C₁₂)alkyl; -Cy2-(C₂-C₁₂)alkenyl; and -Cy2-(C₂-C₁₂)alkynyl; being Cy1 a known ring system independently selected from the group consisting of 5- to 7-membered saturated or partially unsaturated carbocyclic or heterocyclic ring; and 5- to 7-membered carbocyclic or heterocyclic aromatic ring; Cy2 a bivalent known ring system independently selected from the group consisting of 5- to 7-membered saturated or partially unsaturated carbocyclic or heterocyclic ring; and 5- to 7-membered carbocyclic or heterocyclic aromatic ring; and being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl.

The term “alkyl” refers to a saturated straight, or branched hydrocarbon chain which contains the number of carbon atoms specified in the description or claims. Examples include, among others, the group methyl, ethyl, propyl, isopropyl, butyl, isobutyl, sec-butyl, and tert-butyl. As disclosed above, the alkenyl groups can be optionally substituted.

The term “alkenyl” refers to a straight or branched hydrocarbon chain which contains the number of carbon atoms specified in the description or claims and having at least one carbon-carbon double bond. Examples include, among others, ethenyl, 2-propenyl, and 3-hexenyl. As disclosed above, the alkenyl groups can be optionally substituted.

The term “alkynyl” refers to a straight or branched hydrocarbon chain which contains the number of carbon atoms specified in the description or claims and having at least one carbon-carbon triple bond. Examples include, among others, ethynyl, 2-propynyl, and 3-hexynyl. As disclosed above, the alkynyl groups can be optionally substituted.

For the purpose of the invention, the term “bivalent” refer to a moiety that is bond to two other moieties.

The term “—(C₁-C₁₂)alkylene-” refers to a bivalent saturated straight, or branched hydrocarbon chain which contains the number of carbon atoms specified in the description or claims.

The term “—(C₂-C₁₂)alkenylene-” refers to a bivalent straight or branched hydrocarbon chain which contains the number of carbon atoms specified in the description or claims and having at least one carbon-carbon double bond.

The term “—(C₂-C₁₂)alkynylene-” refers to a bivalent straight or branched hydrocarbon chain which contains the number of carbon atoms specified in the description or claims and having at least one carbon-carbon triple bond.

The term “carbocyclic ring” refers to a known saturated, partially unsaturated; or aromatic ring system comprising one or more rings and having the number of carbon atoms specified in the description or claims, wherein all ring members are carbon atoms. As disclosed above, the ring members can be optionally substituted.

The term “heterocyclic ring” refers to a known saturated, partially unsaturated; or aromatic ring system comprising one or more rings and having the number of carbon atoms specified in the description or claims, wherein one or more of the ring members, preferably 1, 2, 3, or 4 ring members, are selected from NH, N, O, and S, and are chemically possible; and the remaining members of the ring are carbon atoms. In the case of a ring system containing a CH member and a NH member, the ring may be attached to the rest of the molecule through the C or the N atom. As disclosed above, the ring members can be optionally substituted.

Non limiting examples of Cy1 include, but not limited to, phenyl and naphthyl, furan-2-yl, furan-3-yl, thiophen-2-yl, thiophen-2-yl, indol-2-yl, imidazolyl, isothiazolyl, isoxazolyl, oxazolyl, pyrazolyl, 2-pyridyl, 3-pyridyl, pyrrolyl, tetrazolyl, thiadiazolyl, thiazolyl, and triazolyl.

The terms “amine protecting group” and “nitrogen protecting group” have the same meaning and are used interchangeable. They refer to a chemical compound which, when bound to an amino group of the polymer or of a starting material (reagent) for its preparation, prevents undesired reactions from occurring at this amino group and which can be removed by conventional chemical or enzymatic steps to re-establish the amino group. Examples of amine protecting groups include carbamate protecting group including, without limitation, t-butyl carbamate, methyl carbamate, ethyl carbamate, 2,2,2-trichloroethyl carbamate, 2-(trimethylsilyl)ethyl carbamate, 1,1-dimethyl-2,2,2-trichloroethyl carbamate, benzyl carbamate, p-methoxybenzyl carbamate, p-nitrobenzylcarbamate, p-bromobenzyl carbamate, p-chlorobenzyl carbamate, and 2,4-dichlorobenzyl carbamate; -Fluorenylmethyl Carbamate (“Fmoc”), formyl, acetyl, trifluoroacetyl, benzyl, benzyloxycarbonyl (“Cbz”), t-butoxycarbonyl (“BOC”), trimethylsilyl (“TMS”), 2-trimethylsilyethanesulfonyl, (“SES”), trityl and substituted trityl groups, allyloxycarbonyl, nitroveratryloxycarbonyl (“NVOC”), and allyloxycarbonyl (Alloc). (cf. Greene and Wuts, Protecting Groups in Organic Synthesis, chapter: “Protection of amines”. Third Edition, John Wiley & Sons (1999) pages 494-653).

The terms “alcohol protecting group” and “oxygen protecting group” have the same meaning and are used interchangeable. They refer to a chemical compound which, when bound to an alcohol group of the polymer or of a starting material (reagent) for its preparation, prevents undesired reactions from occurring at this alcohol group and which can be removed by conventional chemical or enzymatic steps to re-establish the alcohol group. Examples of alcohol protecting groups include 9-Fluorenylmethyl, methoxymethyl, methylthiomethyl, tetrahydrofuranyl, Methoxyethoxymethyl, 2-(Trimethylsilyl)ethoxymethyl, Benzyloxymethyl, Phenylacetoxymethyl, Triisopropylsilylmethyl, Cyanomethyl, Phenacyl, 2,2,2-Trichloroethyl, 2-(Trimethylsilyl)ethyl, 2-Methylthioethyl, 2-(pNitrophenylsulfenyl)ethyl, 2-(pToluenesulfonyl)ethyl, t-Butyl, 2,6-Dimethylphenyl, p(Methylthio)phenyl, Pentafluorophenyl, Benzyl, Nitrobenzyl, p-Nitrobenzyl, p-Methoxybenzyl, 4-Sulfobenzyl, Trimethylsilyl, t-Butyldimethylsilyl, i-Propyldimethylsilyl, Phenyldimethylsilyl, Di-t-butylmethylsilyl, 2-Alkyl-I,3-oxazoline, and Tetraalkylammonium salts. (cf. Greene and Wuts, Protecting Groups in Organic Synthesis, chapter: “Protection of carboxyl group”. Third Edition, John Wiley & Sons (1999) pages 369-453).

Further, the term “detection moiety” refers to a moiety possessing a property or function which can be used for detection purposes. This term encompasses moieties selected from the group consisting of chromophore moiety, fluorescent moiety, phosphorescent moiety, luminescent moiety, light absorbing moiety, radioactive moiety, and transition metal isotope mass tag moiety. The term “chromophore” or “chromophore moiety” refers to a moiety that exhibits a detectable absorption of light such as for example (4,4-difluoro-4-bora-3a,4a-diaza-s-indacene) (BOPIPY). Suitable fluorescent moieties are those known from the art of immunofluorescence technologies, e.g., flow cytometry or fluorescence microscopy, wherein, the compound labelled with this detection moiety is detected by exciting the detection moiety and detecting the resulting emission (photoluminescence). Useful fluorescent moieties for the present invention include protein-based, such as phycobiliproteins, polymeric, such as polyfluorenes, small organic molecule dyes, such as xanthene, like fluorescein, or rhodamines, cyanine, oxazines, coumarins, acridines, oxadiazoles, pyrenes, pyrromethene, or metallo-organic complexes, such as Ru, Eu, Pt complexes. Besides single molecule entities, clusters of fluorescent proteins or small organic molecule dyes, as well as nanoparticles, such as quantum dots, upconverting nanoparticles, gold nanoparticles, dyed polymer nanoparticles can also be used as fluorescent moieties. Another group of photoluminescent detection moieties are phosphorescent moieties with time-delayed emission of light after excitation. Phosphorescent moieties include metallo-organic complexes, such as Pd, Pt, Tb, Eu complexes, or nanoparticles with incorporated phosphorescent pigments such as lanthanide doped SrAl₂O₄. Other group of detection moiety is a radioactive label, wherein the compound labelled with this detection moiety is detected without prior excitation by irradiation. They can be in the form of radioisotope labelling by exchanging nonradioactive isotopes for their radioactive counterparts, such as tritium, ³²P, ³⁵S or ¹⁴C, or introducing covalently bound labels, such as ¹²⁵I, which is bound to tyrosine, ¹⁸F within fluorodeoxyglucose, or metallo-organic complexes, i.e. ⁹⁹Tc-DTPA. Other group is a detection moiety capable of causing chemiluminescence, i.e. horseradish peroxidase label in the presence of luminol. In other group of detection moiety, the labelled compound is detected by absorption of UV, visible light, or NIR radiation. Suitable light-absorbing detection moieties are light absorbing dyes without fluorescence emission, such as small organic molecule quencher dyes like N-aryl rhodamines, azo dyes, and stilbenes. Other detection moiety appropriate for the present invention are light-absorbing detection capable of generating a photoacoustic signal after irradiation by pulsed laser light. In other group of detection moiety, the labelled compound is detected by mass spectrometric detection of a transition metal isotope. Known in the art are isotope tags of lanthanides and adjacent late transition elements.

For the purpose of the invention, the term active moiety is a moiety possessing therapeutically (pharmaceutical or veterinary) or cosmetic activity. This term encompasses moieties selected from the group consisting of a pharmaceutically active ingredient, a veterinary active ingredient, and a cosmetic active ingredient. The term “active ingredient” refers to any chemical compound or substance that has activity in the pharmaceutical, veterinary or cosmetic field. The term “pharmaceutically active ingredient” refers to any substance or combination of substances used in a finished pharmaceutical product, intended to furnish pharmacological activity or to otherwise have direct effect in the cure, mitigation, treatment, or prevention of disease, or to have direct effect in restoring, correcting, or modifying physiological functions in human beings. The term “veterinary active ingredient” refers to any substance or combination of substances used in a finished veterinary product, intended to furnish pharmacological activity or to otherwise have direct effect in the cure, mitigation, treatment, or prevention of disease, or to have direct effect in restoring, correcting, or modifying physiological functions in animals. The term “cosmetic active ingredient” refers to any substance or combination of substances used in a finished cosmetic product, intended to improve its appearance or to beautify, preserve, condition, cleanse, color or protect the skin, nails, or hair without non-medical application.

In an embodiment, the polymer is one wherein one or more of Z and Z′ is selected from an active ingredient and a detection moiety, particularly as defined above. In an embodiment, the polymer is one which the active ingredient and/or the detection moiety are directly bound to the nitrogen atom of the backbone, or alternatively, the polymer is one which the active ingredient and/or the detection moiety bound to the nitrogen atom of the backbone through a linker (positioned between the backbone of the polymer and the detection/active moiety). Examples of appropriate linkers include, but are not limited to, alkylene, anhydrides, alcohols, acids, amines, epoxies, isocyanates, silanes, halogenated groups, and polymerizable groups. The use of a linker and the type of the linker, as well as the specific reaction conditions for bounding the detection and/or active moiety can be readily determined by those skilled in the art according to the type of polymer being prepared. For example, a direct reaction of an activated group on the detection/active agent with the linker (attached to the backbone) or either directly to the atom of the backbone is possible; or vice versa.

As it is mentioned above, the first aspect of the invention is a polymer whose structure is composed of multiple repeating units. The term “polymer” encompasses homopolymers and copolymers regarding if all the repeating units are equal or different, respectively. The term “homopolymer” refers to polymers obtained by polymerization of only one kind of monomer, thereby the repeating units are equal. And the term “copolymer” refers to polymers obtained by polymerization of two or more different kinds of monomers, thereby the repeating units are differents. Particularly, the copolymers can be divided into “block copolymers” or “random copolymers” the term “block copolymer” refers to a polymer comprising two or more homopolymer subunits linked by covalent bonds. Therefore, a block copolymer is made of blocks of different polymerized monomers. Meanwhile, the “random copolymer” refers to a polymer comprising two or more monomers that are distributed randomly throughout the polymer without forming blocks.

In an embodiment, the polymer of the present invention is one wherein the repeating structural units are equal or different of formula (II) or a salt thereof, and n is an integer from n is an integer from 20 to 400. In an embodiment, the polymer of the present invention is one wherein the repeating structural units are equal or different of formula (II) or a salt thereof, and n is an integer from n is an integer from 20 to 230. In an embodiment, the polymer of the present invention is one wherein the repeating structural units are equal or different of formula (II) or a salt thereof, and n is an integer from n is an integer from 20 to 180. In an embodiment, the polymer of the present invention is one wherein the repeating structural units are equal or different of formula (II) or a salt thereof, and n is an integer from n is an integer from 20 to 133. In an embodiment, the polymer of the present invention is one wherein the repeating structural units are equal or different of formula (II) or a salt thereof, and n is an integer from n is an integer from 26 to 133. In an embodiment, the polymer of the present invention is one wherein the repeating structural units are equal or different of formula (II) or a salt thereof, and n is an integer from n is an integer from 20 to 100. In an embodiment, the polymer of the present invention is one wherein the repeating structural units are equal or different of formula (II) or a salt thereof, and n is an integer from n is an integer from 12 to 100. In an embodiment, the polymer of the present invention is one wherein the repeating structural units are equal or different of formula (II) or a salt thereof, and n is an integer from n is an integer from 10 to 150.

For the purpose of the first aspect of the present invention, when m is 0, then the % by weight of the monomer of formula A or a salt thereof is 100% and n is an integer from 20 to 400; and when m is 1, then the percentage of formula A or a salt thereof is 50%, and the percentage of formula B or a salt thereof is 50% and n is an integer from 10 to 400.

For the purpose of the polymer of the first aspect of the invention, the polymer has a weight average molecular weight (Mw) from 2000 to 91000 Da; with the proviso that when the % by weight of monomer A is 100%, then the weight average molecular weight (Mw) of the polymer is from 4500 to 91000 Da. In an embodiment, the aminosquaramide polymer of the first aspect of the invention has a weight average molecular weight (Mw) from 5000 to 70000 Da. In an embodiment, the aminosquaramide polymer of the first aspect of the invention has a weight average molecular weight (Mw) from 5000 to 50000 Da. In an embodiment, the aminosquaramide polymer of the first aspect of the invention has a weight average molecular weight (Mw) from 5000 to 30000 Da. In an embodiment, the aminosquaramide polymer of the first aspect of the invention has a weight average molecular weight (Mw) from 6000 to 30000 Da. In an embodiment, the aminosquaramide polymer of the first aspect of the invention has a weight average molecular weight (Mw) from 6000 to 20000 Da. In an embodiment, the aminosquaramide polymer of the first aspect of the invention has a weight average molecular weight (Mw) from 6000 to 30000 Da.

The terms “weight average molecular weight”, “weight average molar weight” and the abbreviature “Mw” have the same meaning and they are used interchangeable. Mw is a way of determining the molecular weight of a polymer. Polymer molecules, even ones of the same type, come in different sizes (chain lengths, for linear polymers), so the average molecular weight will depend on the method of averaging. It is determined by summing all molecular weights of the fractions of the polymer multiplied by their weight fractions:

$$\overline{{M}_{w}} = \frac{{Σ}_{i = 1}^{N} {N}_{i} {M}_{i}^{2}}{{Σ}_{i = 1}^{N} {N}_{i} {M}_{i}} = \frac{{Σ}_{i = 1}^{N} {w}_{i} {M}_{i}}{{Σ}_{i = 1}^{N} {w}_{i}} = {\sum }_{i = 1}^{N} {w}_{i} {M}_{i}$$

wherein wᵢ is the number of molecules having the molecular weight Mi.

The weight average molecular weight of a polymer can be determined by any method known in the state of the art for instance gel permeation chromatography (GPC), viscometry via the (Mark-Houwink equation), colligative methods such as vapor pressure osmometry, analytical size-exclusion chromatography, and refractive index detector (SEC/MALS/RI), and end-group determination or proton NMR. For the purpose of the present invention, the measurement of the weight average molecular weight (Mw) of aminosquaramide polymers having from 2000 to 30000 Da was performed by Gel Permeation Chromatography-Visible Ultraviolet (GPC-UV); and of aminosquaramide polymers having from higher than 30000 to 91000 Da was performed by analytical size-exclusion chromatography coupled with multi-angle light scattering and refractive index detector (SEC/MALS/RI).

The terms “number average molecular weight”, “number average molar mass” and the abbreviature Mn” have the same meaning and they are used interchangeable. Mn is a way of determining the molecular mass of a polymer. Polymer molecules, even ones of the same type, come in different sizes (chain lengths, for linear polymers), so the average molecular mass will depend on the method of averaging. The number average molecular mass is the ordinary arithmetic means or average of the molecular masses of the individual macromolecules. It is determined by measuring the molecular mass of n polymer molecules, summing the masses, and dividing by n. The Mn is calculated by the following formula:

$${\overline{M}}_{n} = \frac{{\sum }_{i} {N}_{i} {M}_{i}}{{\sum }_{i} {N}_{i}}$$

wherein Nᵢ is the number of molecules of molecular mass Mi.

The number average molecular mass of a polymer can be determined by gel permeation chromatography (GPC), viscometry via the (Mark-Houwink equation), colligative methods such as vapor pressure osmometry, end-group determination or proton NMR. For the purpose of the invention the Mn is measured by GPC.

In an embodiment, the aminosquaramide polymer comprising a backbone of repeating structural units, being these repeating structural units equal or different of formula (II) or a salt thereof, wherein m is 0; thereby the repeating units are equal or different of formula (II)-A

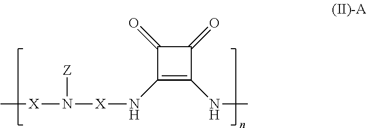

being Z and n as defined in formula (II).

In an embodiment, the aminosquaramide polymer comprising a backbone of repeating structural units, being these repeating structural units equal of formula (II)-A. In an embodiment, the aminosquaramide polymer comprising a backbone of repeating structural units, being these repeating structural units different of formula (II)-A, distributed by block or randomly.

In an embodiment, the aminosquaramide polymer is one wherein: in the repeating structural units, being these repeating structural units equal or different of formula (II) or a salt thereof, m is 0 and thereby the aminosquaramide polymer is an aminosquaramide polymer (I) selected from the group consisting of:
a polymer of formula (I)-1A or a salt thereof;

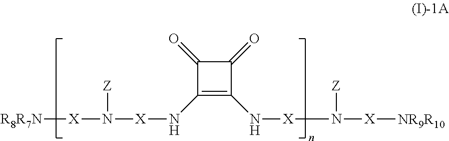

a polymer of formula (I)-1B, or a salt thereof;

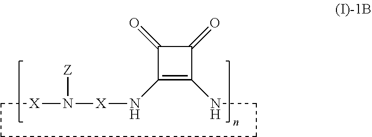

and
a mixture of one or more polymers of formula (I)-1A, or a salt thereof; and one or more polymers of formula (I)-1B, or a salt thereof;
wherein:
X, Y, Z, Z′, R₁, R₂, R₃, R₄, R₅, R₆, halogen, n, p, q, and r are as defined above;
dashed line [symbol] is a single bond that bonds X and NH;
R₇, R₈, R₉ and R₁₀ are independently selected from the group consisting of H, an amine protecting group, a detection moiety, an active moiety, and a moiety of formula (III):

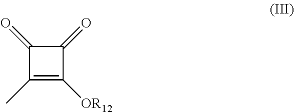

being:
the detection moiety as defined in claim 1;
R₁₂ is selected from the group consisting of —(C₁-C₆)alkyl, H and an alcohol protecting group;
with the proviso that:
when one of R₇ and R₈ is a detection moiety, or an active moiety, the other is selected from H and alcohol protecting group; and
when one of R₉ and R₁₀ is a detection moiety, or an active moiety, the other is selected from H and alcohol protecting group.

In an embodiment, the aminosquaramide polymer is one wherein the aminosquaramide polymer is an aminosquaramide polymer (I) selected from the group consisting of a polymer of formula (I)-1A, or a salt thereof; a polymer of formula (I)-1B, or a salt thereof; and a mixture of one or more polymers of formula (I)-1A, or a salt thereof; and one or more polymers of formula (I)-1B, or a salt thereof; wherein all repeating units are equal.

In an embodiment, the aminosquaramide polymer is one wherein the aminosquaramide polymer is an aminosquaramide polymer (I) selected from the group consisting of a polymer of formula (I)-1A, or a salt thereof; a polymer of formula (I)-1B, or a salt thereof; and a mixture of one or more polymers of formula (I)-1A, or a salt thereof; and one or more polymers of formula (I)-1B, or a salt thereof; wherein: all repeating units are equal; and X is —(CH₂)_{q}—; particularly Z is an inert moiety such as —CH₃.

In an embodiment, the aminosquaramide polymer is one wherein the aminosquaramide polymer is an aminosquaramide polymer (I) selected from the group consisting of a polymer of formula (I)-1A, or a salt thereof; a polymer of formula (I)-1B, or a salt thereof; and a mixture of one or more polymers of formula (I)-1A, or a salt thereof; and one or more polymers of formula (I)-1B, or a salt thereof; wherein: all repeating units are equal; and —(CH₂)₃—; particularly Z is an inert moiety such as —CH₃.

In an embodiment, the aminosquaramide polymer comprising a backbone of repeating structural units, being these repeating structural units equal or different of formula (II) or a salt thereof, wherein m is 1; thereby the repeating units are equal or different of formula (II)-B

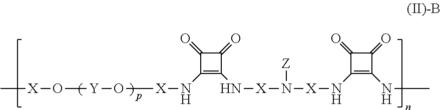

being Z and n as defined in formula (II).

In an embodiment, the aminosquaramide polymer comprising a backbone of repeating structural units, being these repeating structural units equal of formula (II)-B. In an embodiment, the aminosquaramide polymer (I) comprising a backbone of repeating structural units, being these repeating structural units different of formula (II)-B, distributed by block or randomly.

In an embodiment, the aminosquaramide polymer (I) is one wherein: in the repeating structural units, being these repeating structural units equal or different of formula (II) or a salt thereof, m is 1 and thereby the aminosquaramide polymer is an aminosquaramide polymer (I) selected from the group consisting of:
a polymer of formula (I)-2A, or a salt thereof;

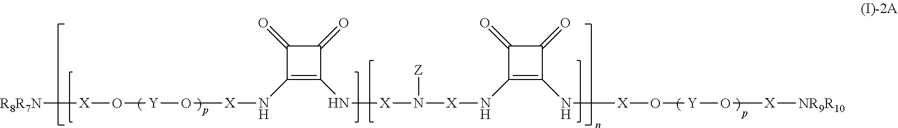

a polymer of formula (I)-2B, or a salt thereof;

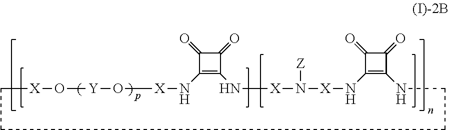

and
a mixture of one or more polymers of formula (I)-2A, or a salt thereof; and one or more polymers of formula
(I)-2B, or a salt thereof;
wherein:
X, Y, Z, Z′, R₁, R₂, R₃, R₄, R₅, R₆, halogen, n, p, q, and r are as defined in claim 1;
dashed line [symbol] is a single bond that bonds X and NH;
R₇, R₈, R₉ and R₁₀ are independently selected from the group consisting of H, an amine protecting group, a detection moiety, an active moiety, and a moiety of formula (III):

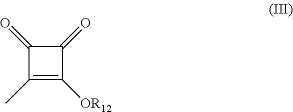

being:
the detection moiety as defined in claim 1;
R₁₂ is selected from the group consisting of —(C₁-C₆)alkyl, H and an alcohol protecting group;
with the proviso that:
when one of R₇ and R₈ is a detection moiety, or an active moiety, the other is selected from H and alcohol protecting group; and
when one of R₉ and R₁₀ is a detection moiety, or an active moiety, the other is selected from H and alcohol protecting group.

In an embodiment, the aminosquaramide polymer is an aminosquaramide polymer (I) selected from the group consisting of a polymer of formula (I)-2A, or a salt thereof; a polymer of formula (I)-2B, or a salt thereof; and a mixture of one or more polymers of formula (I)-2A, or a salt thereof; and one or more polymers of formula (I)-2B, or a salt thereof; wherein all repeating units are equal.

In an embodiment, the aminosquaramide polymer is an aminosquaramide polymer (I) selected from the group consisting of a polymer of formula (I)-2A, or a salt thereof; a polymer of formula (I)-2B, or a salt thereof; and a mixture of one or more polymers of formula (I)-2A, or a salt thereof; and one or more polymers of formula (I)-2B, or a salt thereof; wherein: all repeating units are equal; X is —(CH₂)_{q}—; and Y is —(CH₂)ᵣ—; particularly Z is an inert moiety such as —CH₃.

In an embodiment, the aminosquaramide polymer an aminosquaramide polymer (I) selected from the group consisting of a polymer of formula (I)-2A, or a salt thereof; a polymer of formula (I)-2B, or a salt thereof; and a mixture of one or more polymers of formula (I)-2A, or a salt thereof; and one or more polymers of formula (I)-2B, or a salt thereof; wherein: all repeating units are equal; X is —(CH₂)₃—; Y is —(CH₂)₂—; R₅ and R₆ are H, and p is 2; particularly Z is an inert moiety such as —CH₃.

In an embodiment, the aminosquaramide polymer an aminosquaramide polymer (I) selected from the group consisting of a polymer of formula (I)-1A, or a salt thereof; a polymer of formula (I)-1B, or a salt thereof; and a mixture of one or more polymers of formula (I)-1A, or a salt thereof; and one or more polymers of formula (I)-1B, or a salt thereof; wherein the repeating units are different; distributed by block or randomly.

In an embodiment, the aminosquaramide polymer is an aminosquaramide polymer (I) selected from the group consisting of a polymer of formula (I)-1A, or a salt thereof; a polymer of formula (I)-1B, or a salt thereof; and a mixture of one or more polymers of formula (I)-1A, or a salt thereof; and one or more polymers of formula (I)-1B, or a salt thereof; wherein: the repeating units are different distributed by block or randomly; X is —(CH₂)_{q}—; Z is —CH₃ in a percentage of repeating structural units from 50 to 90% in relation to the total weight of the polymer (I) or a salt thereof; and Z is other than —CH₃ in a percentage of repeating structural units from 10 to 50% in relation to the total weight of the polymer (I) or a salt thereof; particularly the Z other than —CH₃ is a detection or active moiety.

In an embodiment, the aminosquaramide polymer is an aminosquaramide polymer (I) selected from the group consisting of a polymer of formula (I)-1A, or a salt thereof; a polymer of formula (I)-1B, or a salt thereof; and a mixture of one or more polymers of formula (I)-1A, or a salt thereof; and one or more polymers of formula (I)-1B, or a salt thereof; wherein: the repeating units are different distributed by block or randomly; X is —(CH₂)₃—; Z is —CH₃ in a percentage of repeating structural units from 50 to 90% in relation to the total weight of the polymer (I) or a salt thereof; and Z is other than —CH₃ in a percentage of repeating structural units from 10 to 50% in relation to the total weight of the polymer (I) or a salt thereof; particularly the Z other than —CH₃ is a detection or active moiety.

In an embodiment, the aminosquaramide polymer is an aminosquaramide polymer (I) selected from the group consisting of a polymer of formula (I)-2A, or a salt thereof; a polymer of formula (I)-2B, or a salt thereof; and a mixture of one or more polymers of formula (I)-2A, or a salt thereof; and one or more polymers of formula (I)-2B, or a salt thereof; wherein: the repeating units are different distributed by block or randomly; X is —(CH₂)_{q}—; and Y is —(CH₂)ᵣ—; Z is —CH₃ in a percentage of repeating structural units from 50 to 90% in relation to the total weight of the polymer (I) or a salt thereof; and Z is other than —CH₃ in a percentage of repeating structural units from 10 to 50% in relation to the total weight of the polymer (I) or a salt thereof; particularly the Z other than —CH₃ is a detection or active moiety.

In an embodiment, the aminosquaramide polymer is an aminosquaramide polymer (I) selected from the group consisting of a polymer of formula (I)-2A, or a salt thereof; a polymer of formula (I)-2B, or a salt thereof; and a mixture of one or more polymers of formula (I)-2A, or a salt thereof; and one or more polymers of formula (I)-2B, or a salt thereof; wherein: the repeating units are different distributed by block or randomly; X is —(CH₂)₃—; Y is —(CH₂)₂—; R₅ and R₆ are H, and p is 2; Z is —CH₃ in a percentage of repeating structural units from 50 to 90% in relation to the total weight of the polymer (I) or a salt thereof; and Z is other than —CH₃ in a percentage of repeating structural units from 10 to 50% in relation to the total weight of the polymer (I) or a salt thereof; particularly the Z other than —CH₃ is a detection or active moiety.

In an embodiment, the aminosquaramide polymer is an aminosquaramide polymer of formula (I)-1A and of formula (I)-2A as defined in the present invention wherein: R₇, R₈, R₉ and R₁₀ are independently selected from the group consisting of H and an amine protecting group. In an embodiment, the aminosquaramide polymer is an aminosquaramide polymer of formula (I)-1A and of formula (I)-2A as defined in the present invention wherein: all R₇, R₈, R₉ and R₁₀ are H.

In an embodiment, the aminosquaramide polymer is an aminosquaramide polymer of formula (I)-1A and of formula (I)-2A as defined in the present invention wherein: R₇, R₈, R₉ and R₁₀ are independently selected from the list consisting of a detection moiety and an active moiety as defined in the present invention, with the proviso that: when one of R₇ and R₈ is a detection moiety, an active moiety, the other is selected from H and alcohol protecting group; and when one of R₉ and R₁₀ is a detection moiety or an active moiety, the other is selected from H and alcohol protecting group.

In an embodiment, the aminosquaramide polymer of the invention is one wherein: the percentage of monomer of formula B or a salt thereof is different than zero, and the monomers of formula A or a salt thereof and of formula B or a salt thereof are randomly distributed. In an embodiment, the aminosquaramide polymer of the invention is one wherein: the percentage of monomer of formula B or a salt thereof is different than zero, the monomers of formula A or a salt thereof and of formula B or a salt thereof are randomly distributed; and the percentage by weight of the monomer of formula A or a salt thereof is equal to or higher than 80%; being the sum equal to 100%. In an embodiment, the aminosquaramide polymer of the invention is one wherein: the percentage of monomer of formula B or a salt thereof is different than zero, the monomers of formula A or a salt thereof and of formula B or a salt thereof are randomly distributed; and the percentage by weight of the monomer of formula A or a salt thereof is equal to or higher than 85%; being the sum equal to 100%. In an embodiment, the aminosquaramide polymer of the invention is one wherein: the percentage of monomer of formula B or a salt thereof is different than zero, the monomers of formula A or a salt thereof and of formula B or a salt thereof are randomly distributed; and the percentage by weight of the monomer of formula A or a salt thereof is equal to or higher than 90%; being the sum equal to 100%. In an embodiment, the aminosquaramide polymer of the invention is one wherein: the percentage of monomer of formula B or a salt thereof is different than zero, the monomers of formula A or a salt thereof and of formula B or a salt thereof are randomly distributed; and the percentage by weight of the monomer of formula A or a salt thereof is equal to or higher than 95%; being the sum equal to 100%.

It is also a part of the invention a process for the preparation of the aminosquaramide polymers of the first aspect of the invention. The appropriate reagents and theirs amounts as well as the reaction conditions (for example temperature, time, and solvents), can be determined by those skilled in the art according to the aminosquaramide polymer being prepared. The above mentioned aminosquaramide polymers can be prepared according to polymerizing methods well known in the state of the art.

As example, aminosquaramide polymers of formula (I)-1A-1 and of formula (I)-1B-1 of the present invention wherein m is 0 and the respecting units of formula (II) are equal, thereby all X are equal and all Z are equal being an inert moiety can be obtained by the following process comprising reacting the reagent 1 and the reagent 2 under such reaction conditions to obtain the aminosquaramide polymer of formula (I)-1A-1 Ia (I)-1B-1:

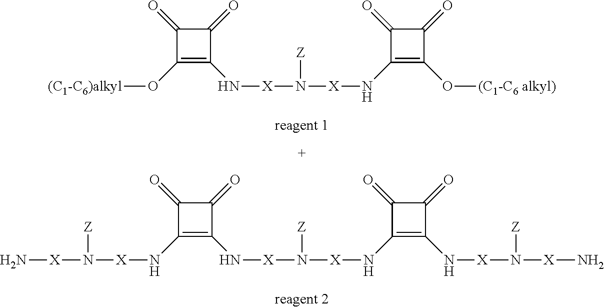

or alternatively, by reacting reagent 3 and reagent 4 as disclosed herein below, under such reaction conditions to obtain the aminosquaramide polymer of formula (I)-1A-1 and of formula (I)-1B-1:

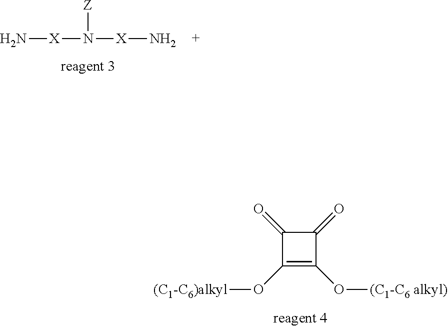

As other example, aminosquaramide polymers of formula (I)-1A-2 and of formula (I)-1B-2 wherein m is 0 and the repeating units are different, being X as defined in the first aspect of the invention; Z1 and Z2 are different; and Z1 and Z2 are as defined in the first aspect of the invention for Z or Z′, can be obtained by the following process comprising reacting reagent 5 and reagent 6 herein below, under such reaction conditions to obtain the aminosquaramide polymer of formula (I)-1A-2 and (I)-1B-2:

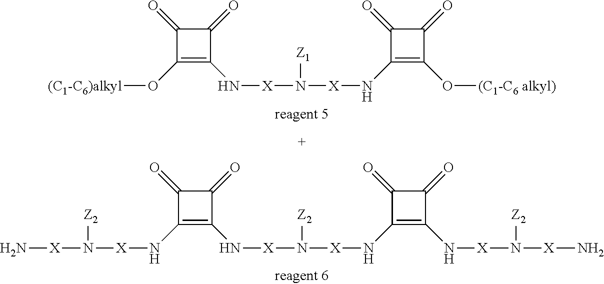

or alternatively, by reacting reagent 4 and reagent 7; and the resulting mixture thus obtained is mixed with reagent 8 as disclosed herein below under such reaction conditions to obtain the aminosquaramide polymer of formula (I)-1A-2 and (I)-1B-2:

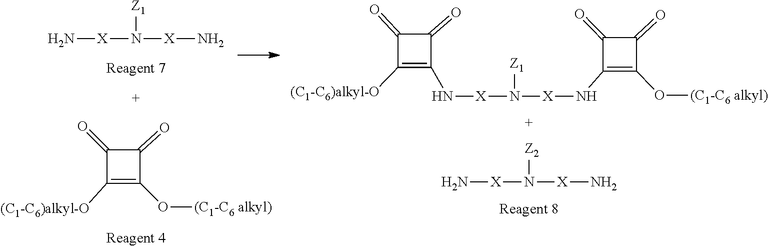

As other example, aminosquaramide polymers of formula (I)-1A-1 and of formula (I)-1B-1 of the present invention wherein m is 0 and the repeating units of formula (II) are equal, thereby all X are equal and all Z are equal being an inert moiety can be obtained by the following process comprising reacting the reagent 7 and the reagent 5 under such reaction conditions to obtain the aminosquaramide polymer of formula (I)-1A-1 Ia (I)-1B:

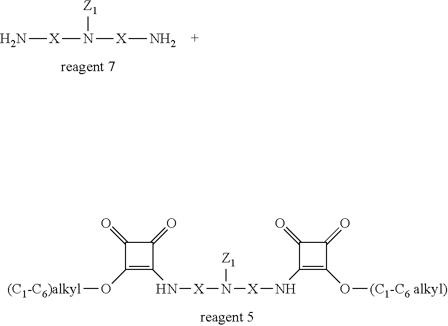

In an embodiment, wherein one or more of Z1 and Z2 are selected from a detection moiety or an active moiety then the detection moiety and the active moiety (as aminosquaramide polymers of formula (I)-1A-3 and (1)-1B-3) can already be present in the starting materials (reagents 5-8) disclosed above; or alternatively can be introduced in one or more additional subsequent steps. In an embodiment, when the detection moiety and/or active moiety are introduced in a subsequent step, the process comprises, first reacting a reagent 5 with a reagent 6 wherein Z1 and Z2 are inert moieties (or alternatively reacting reagent 7 and reagent 4 and subsequent with reagent 8 wherein Z1 and Z2 are inert moieties) and then contacting the mixture thus obtained with the detection compound and/or the active compound under such reaction conditions to achieve the bounding of the active or detection compound to the aminosquaramide polymer.

As other example, polymers of formula (I)-1A-4 and of formula (I)-1B-4, wherein m is 0 and the repeating units are different, being X is as defined in the first aspect of the invention; Z1 and Z2 are different; and Z1 and Z2 are as defined in Z, can be obtained by the following process comprising reacting reagent 7 and reagent 8 with reagent 4 as disclosed herein below under such reaction conditions to obtain the aminosquaramide polymer of formula (I)-1A-4 and (I)-1B-4:

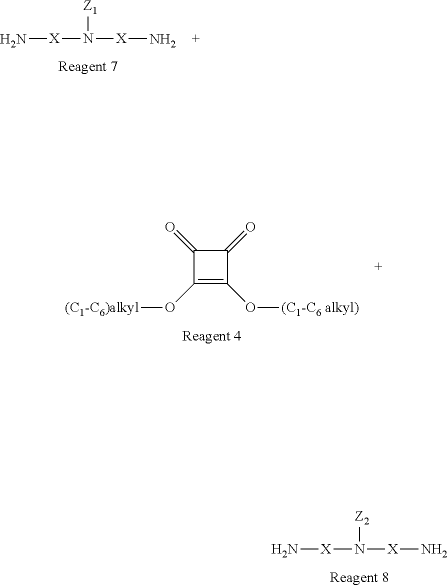

In an embodiment, wherein one or more of Z1 and Z2 are selected from a detection moiety or an active moiety then the detection moiety and the active moiety can already be present in the starting materials (reagent 7 and/or reagent 8) disclosed above; or alternatively can be introduced in one or more additional subsequent steps. In an embodiment, when the detection moiety and/or active moiety are introduced in a subsequent step, the process comprises, first reacting reagent 7, reagent 8 with reagent 4 wherein Z1 and Z2 are inert moieties and subsequent reacting the mixture thus obtained with the detection compound and/or the active compound under such reaction conditions to achieve the bounding of the active or detection compound to the aminosquaramide polymer.

As other example, the polymers of formula (I)-2A-1 and of formula (I)-2B-1, wherein m is 1 and the repeating units are equal, being X and Z as defined in the first aspect of the invention can be obtained by the following process comprising reacting reagent 9 and reagent 10 as disclosed herein below under such reaction conditions to obtain the aminosquaramide polymer of formula (I)-2A-1 and of formula (I)-2A-1:

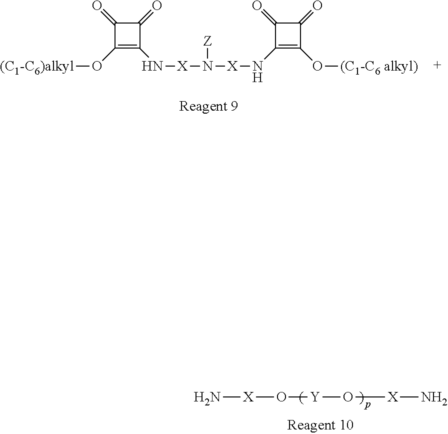

In an embodiment, the processes disclosed herein above for the preparation of the aminosquaramide polymers of the present invention further comprises a previous step of deprotecting the amino-protecting groups of amine moieties of reagents 1-10.

In an embodiment, the processes disclosed herein above for the preparation of the aminosquaramide polymers of the present invention further comprises an additional purifying step and/or fractionating step. The purifying and fractionating steps can be performed by any method in the state of the art for polymers. For the purpose of the present invention, the purification and/or fractioning step can be performed by repeating cycles of a re-dissolution/precipitation-filtration process (at pH 3 for the re-dissolution steps and from 7.4-8.1 for the re-precipitation steps) or centrifugal filters of 3 and 10 kDa MW cut-offs for the medium and high molecular weight fractions, respectively.

Examples of appropriate solvents for performing the preparation processes disclosed herein above and below for the aminosquaramide polymers of the present invention include, but it is not limited to, (C₁-C₅)alcohols such as ethanol and methanol; dimethylsulphoxide (DMSO) and dimethylformamide (DMF). The term “alcohol” refers to an “alkane” wherein at least one hydrogen atom is substituted by a hydroxyl group and which contains the number of carbon atoms specified in the description or claims. The term “alkane” refers to a saturated, branched, or linear hydrocarbon which contains the number of carbon atoms specified in the description or claims.

These processes can be performed at a temperature from room temperature to below the boiling point of the solvent. The term “room temperature” refers to a temperature of the environment, without heating or cooling, and it is generally comprised from 20° C. to 25° C. For example, the processes disclosed in the present invention can be performed using ethanol as solvent at a temperature from 20 to 78° C.; or in the sinus of dimethyl sulfoxide at a temperature from 20-140° C., for the appropriate time for obtaining the aminosquaramide polymer of the present invention. By way of example, a process for the preparation of an aminosquaramide polymer of the present invention having a weight average molecular weight (Mw) from 2000 to 30000 Da can be performed following any one of the methods A-C as defined above using; particularly in the sinus of (C₁-C₅) alcohol (particularly ethanol) as a solvent and at a temperature from 20-78° C. And a process for the preparation of an aminosquaramide polymer of the present invention having a weight average molecular weight (Mw) from 30000 to 91000 Da can be performed following the method D as defined above using dimethyl sulfoxide as a solvent and at a temperature from 20-140° C.

It is also part of the invention an aminosquaramide polymer characterized by its preparation process. Therefore, the aminosquaramide polymers of the present invention obtainable by the processes disclosed above in the present application are also part of the invention. For the purposes of the invention the expressions “obtainable”, “obtained” and equivalent expressions are used interchangeably, and in any case, the expression “obtainable” encompasses the expression “obtained”.

The second aspect of the invention is a conjugate comprising the aminosquaramide polymer comprising a backbone of repeating structural units, being these repeating structural units equal or different and selected from the group consisting of: monomer of formula A or a salt thereof as defined herein above and below and monomer of formula B or a salt thereof as defined herein above and below, wherein:
X is selected from the group consisting of —(CH₂)_{q}—, —(CHR₁)_{q}— and —(CR₁R₂)_{q}—;
Y is selected from the group consisting of —(CH₂)ᵣ—, —(CHR₃)ᵣ— and —(CR₃R₄)ᵣ—;
R₁ and R₂ are independently selected from the group consisting of H and Z′;
R₃ and R₄ are independently selected from the group consisting of H and Z′;
each Z and Z′ are independently selected from the group consisting of an inert moiety, a detection moiety, and an active moiety;
the inert moiety is selected from the group consisting of —(C₁-C₁₂)alkyl; —(C₂-C₁₂)alkenyl; —(C₂-C₁₂)alkylene-O—(Y—O)_{w}—(C₁-C₁₂)alquil, —(C₂-C₁₂)alkylene-O—(Y—O)_{w}—(C₂-C₁₂)alkylene-NR₁₁R₁₃, —(C₂-C₆)alkynyl; -Cy1; —(C₁-C₁₂)alkylene-Cy1; —(C₂-C₁₂)alkenylene-Cy1; —(C₂-C₁₂)alkynylene-Cy1; -Cy2-(C₁-C₁₂)alkyl; -Cy2-(C₂-C₁₂)alkenyl;
and -Cy2-(C₂-C₁₂)alkynyl; being Cy1 a known ring system independently selected from the group consisting of 5- to 7-membered saturated or partially unsaturated carbocyclic or heterocyclic ring; and 5- to 7-membered carbocyclic or heterocyclic aromatic ring; Cy2 a bivalent known ring system independently selected from the group consisting of 5- to 7-membered saturated or partially unsaturated carbocyclic or heterocyclic ring; and 5- to 7-membered carbocyclic or heterocyclic aromatic ring; and being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl; being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl;
R₅ and R₆ are independently selected from the group consisting of —(C₁-C₁₂)alkyl and an amine protecting group;
R₁₁ and R₁₃ are independently selected from the group consisting of H, —(C₁-C₁₂)alkyl and an amine protecting group;
Halogen is selected from the group consisting of F, Cl, Br, and I;
the detection moiety is selected from the group consisting of chromophore moiety, fluorescent moiety, phosphorescent moiety, luminescent moiety, light absorbing moiety, radioactive moiety, transition metal and isotope mass tag moiety; and
the active moiety is selected from the group consisting of a pharmaceutically active ingredient, a veterinary active ingredient, and a cosmetic active ingredient;
p is an integer from 1 to 10;
q is an integer from 2 to 6;
r is an integer from 2 to 4;
the percentage by weight of the monomer of formula A or a salt thereof is from 30% to 100% by weight;
the percentage by weight of the monomer of formula B or a salt thereof is from 0% to 70% by weight;
being the sum of the percentages equal to 100%; and
the aminosquaramide polymer has a weight average molecular weight (Mw) from 2000 to 91000 Da; and
one or more molecules of interest;
being:
the weight average molecular weight (Mw) from 2000 to 30000 Da measured by Gel Permeation Chromatography-Visible Ultraviolet (GPC-UV); and
the weight average molecular weight (Mw) from higher than 30000 to 91000 Da measured by analytical size-exclusion chromatography coupled with multi-angle light scattering and refractive index detector (SEC/MALS/RI).

In an embodiment, the conjugate comprises an aminosquaramide polymer comprising repeating structural units equal or different of formula (II) or a salt thereof,

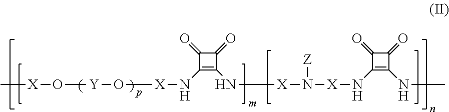

wherein: m is 0 or 1; and n is an integer from 10 to 400; and one or more molecules of interest.

In an embodiment, the conjugate comprises an aminosquaramide polymer comprising repeating structural units equal or different of formula (II) or a salt thereof,

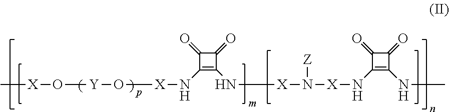

wherein: m is 0 or 1; and n is an integer from 20 to 180; and one or more molecules of interest.

In an embodiment, the conjugate comprises the polymer of the first aspect of the invention and one or more molecules of interest.

For the purpose of the invention, the terms “conjugate” and “complex” have the same meaning and are used interchangeable. They refers to the union of an aminosquaramide polymer of the first aspect of the invention and one or more molecules of interest linked together. The union between the aminosquaramide polymer and the molecule of interest is “non-covalent”. The term “non-covalent” refers to the bond between the aminosquaramide polymer and the molecule of interest that involves weak interactions such as for example ionic interactions, electrostatic interactions, hydrogen bonding and/or van der Waals interactions. The type of interaction (union) mainly depends on the molecule of interest. In particular, the type of union between the aminosquaramide polymer and the molecule of interest can be readily determined by those skilled in the art according to the chemical structure and the physico-chemical properties of the molecule of interest used for the preparation of the conjugate. For the purpose of the invention, the term “conjugate” encompasses the term “polyplex” which refers to a specific conjugate of the present invention comprising an aminosquaramide polymer of the invention and one or more nucleic acid-containing compound as a molecule of interest (also called “cargo”).

For the purpose of the present invention, the term “molecule of interest” encompasses active ingredients, amino acid-containing compounds, nucleic acid-containing compounds, and mixtures thereof.

In an embodiment, the conjugate of the invention is one wherein the one or more molecules of interest comprises at least one active ingredient as defined above.

In an embodiment, the conjugate of the invention is one wherein the one or more molecules of interest comprises at least one amino acid-containing compound. In an embodiment, the conjugate of the invention is one wherein the one or more molecules of interest comprises at least one amino acid-containing compound selected from the group consisting of a polypeptide, a protein and a mixture thereof. The terms “peptide” and “polypeptide” have the same meaning and are used interchangeably. They refer to chains having from 2 to 50 amino acid residues, and the term “protein” refers to chains of more than 50 amino acid residues. In an embodiment, the conjugate of the invention is one wherein the one or more molecules of interest comprises at least an antibody or a fragment thereof as amino acid-containing compound. The term “antibody or a fragment thereof” refers to any immunoglobulin or fragment thereof suitable to bind an epitope of the target protein. It includes monoclonal and polyclonal antibodies. The term “fragment thereof” encompasses any part of an antibody having the size and conformation suitable to bind an epitope of the target protein. Suitable fragments include F(ab), F(ab′) and Fv. An “epitope” is the part of the antigen being recognized by the immune system (B-cells, T-cells or antibodies). Particularly, the antibodies used for specific detection can be polyclonal or monoclonal. There are well known means in the state of the art for preparing and characterizing antibodies. Methods for generating polyclonal antibodies are well known in the prior art. Briefly, one prepares polyclonal antibodies by immunizing an animal with the protein; then, serum from the immunized animal is collected and the antibodies isolated. A wide range of animal species can be used for the production of the antiserum. Typically the animal used for production of antisera can be a rabbit, mouse, rat, hamster, guinea pig or goat. Moreover, monoclonal antibodies (MAbs) can be prepared using well-known techniques. Typically, the procedure involves immunizing a suitable animal with the protein associated with the disease. The immunizing composition can be administered in an amount effective to stimulate antibody producing cells. Methods for preparing monoclonal antibodies are initiated generally following the same lines as the polyclonal antibody preparation. The immunogen is injected into animals as antigen. The antigen may be mixed with adjuvants such as complete or incomplete Freund's adjuvant. At intervals of two weeks, approximately, the immunization is repeated with the same antigen.

For the purpose of the present invention, the terms “nucleic acid-containing compound” and “cargo” have the same meaning and are used interchangeable. The cargo is capable of introducing into cells utilizing the aminosquaramide polymer of the present invention as means of transfection. In an embodiment, the conjugate of the invention is one wherein the one or more molecules of interest comprises at least one nucleic acid-containing compound. In an embodiment, the conjugate of the invention is one wherein the one or more molecules of interest comprises at least one nucleic acid-containing compound selected from the group consisting of single strand oligonucleotides such as DNA, RNA, PNA, LNA and analogues thereof; double-strand oligonucleotides such as siRNA, shRNA, decoy DNA; plasmids and analogues thereof. As it is demonstrated in the experimental section, the aminosquaramide polymer of the present invention can be used as transfecting agent. In fact, it is shown that a polyplex comprising the aminosquaramide polymer of the present invention is capable of conjugate until ten molecules of nucleic acid of interest for transfecting into cells.

All embodiments disclosed above for the aminosquaramide polymer also applies for the conjugate of the second aspect of the invention.

It is also a part of the invention a process for the preparation of the conjugates of the second aspect of the invention. The appropriate reagents and theirs amounts as well as the reaction conditions, can be determined by those skilled in the art according to the conjugate being prepared. Commonly, the process for the preparation of the conjugates comprises contacting the aminosquaramide polymer with the molecule of interest under such reaction conditions that allows the union between them.

Further, it is also part of the invention a conjugate characterized by its preparation process. Therefore, the conjugate of the second aspect of the invention obtainable by the processes disclosed above in the present application are also part of the invention. All embodiments disclosed above for the aminosquaramide polymer of the first aspect of the invention, and the conjugate of the second aspect of the invention also apply here for the conjugate obtainable by its preparation process.

The third aspect of the present invention refers to a composition comprising an aminosquaramide polymer comprising a backbone of repeating structural units, being these repeating structural units equal or different and selected from the group consisting of: monomer of formula A or a salt thereof as defined herein above and below, and monomer of formula B or a salt thereof as defined above and below, wherein:
X is selected from the group consisting of —(CH₂)_{q}—, —(CHR₁)_{q}— and —(CR₁R₂)_{q}—;
Y is selected from the group consisting of —(CH₂)ᵣ—, —(CHR₃)ᵣ— and —(CR₃R₄)ᵣ—;
R₁ and R₂ are independently selected from the group consisting of H and Z′;
R₃ and R₄ are independently selected from the group consisting of H and Z′;
each Z and Z′ are independently selected from the group consisting of an inert moiety, a detection moiety and an active moiety;
the inert moiety is selected from the group consisting of —(C₁-C₁₂)alkyl; —(C₂-C₁₂)alkenyl; —(C₂-C₁₂)alkylene-O—(Y—O)_{w}—(C₁-C₁₂)alquil, —(C₂-C₁₂)alkylene-O—(Y—O)_{w}—(C₂-C₁₂)alkylene-NR₁₁R₁₃, —(C₂-C₆)alkynyl; -Cy1; —(C₁-C₁₂)alkylene-Cy1; —(C₂-C₁₂)alkenylene-Cy1; —(C₂-C₁₂)alkynylene-Cy1; -Cy2-(C₁-C₁₂)alkyl; -Cy2-(C₂-C₁₂)alkenyl; and -Cy2-(C₂-C₁₂)alkynyl; being Cy1 a known ring system independently selected from the group consisting of 5- to 7-membered saturated or partially unsaturated carbocyclic or heterocyclic ring; and 5- to 7-membered carbocyclic or heterocyclic aromatic ring; Cy2 a bivalent known ring system independently selected from the group consisting of 5- to 7-membered saturated or partially unsaturated carbocyclic or heterocyclic ring; and 5- to 7-membered carbocyclic or heterocyclic aromatic ring; and being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl; being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl;
R₅ and R₆ are independently selected from the group consisting of —(C₁-C₁₂)alkyl and an amine protecting group;
R₁₁ and R₁₃ are independently selected from the group consisting of H, —(C₁-C₁₂)alkyl and an amine protecting group;
Halogen is selected from the group consisting of F, Cl, Br and I;
the detection moiety is selected from the group consisting of chromophore moiety, fluorescent moiety, phosphorescent moiety, luminescent moiety, light absorbing moiety, radioactive moiety, transition metal and isotope mass tag moiety; and
the active moiety is selected from the group consisting of a pharmaceutically active ingredient, a veterinary active ingredient and a cosmetic active ingredient;
p is an integer from 1 to 10;
q is an integer from 2 to 6;
r is an integer from 2 to 4;
the percentage by weight of the monomer of formula A or a salt thereof is from 30% to 100% by weight;
the percentage by weight of the monomer of formula B or a salt thereof is from 0% to 70% by weight;
being the sum of the percentages equal to 100%; and
the aminosquaramide polymer has a weight average molecular weight (Mw) from 2000 to 91000 Da
being:
the weight average molecular weight (Mw) from 2000 to 30000 Da measured by Gel Permeation Chromatography-Visible Ultraviolet (GPC-UV); and
the weight average molecular weight (Mw) from higher than 30000 to 91000 Da measured by analytical size-exclusion chromatography coupled with multi-angle light scattering and refractive index detector (SEC/MALS/RI);
or alternatively, the conjugate as defined in the second aspect of the invention,
together with one or more appropriate excipients or carriers.

In an embodiment, the conjugate comprises an aminosquaramide polymer comprising repeating structural units equal or different of formula (II) or a salt thereof,

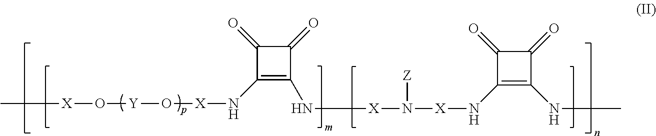

wherein: m is 0 or 1; and n is an integer from 10 to 400, and one or more molecules of interest.

In an embodiment, the conjugate comprises an aminosquaramide polymer comprising repeating structural units equal or different of formula (II) or a salt thereof,

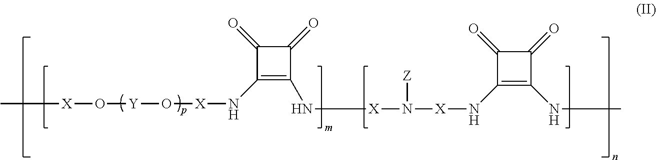

wherein: m is 0 or 1; and n is an integer from 20 to 180, and one or more molecules of interest.

In an embodiment, the conjugate comprises the aminosquaramide polymer as defined in the first aspect of the invention, and one or more molecules of interest.

The appropriate excipients and carriers and their amounts can readily be determined by those skilled in the art according to the type of composition being prepared.

In an embodiment, the composition is a therapeutic composition comprising a therapeutically (pharmaceutical or veterinary) effective amount of one or more active ingredients; and one or more therapeutically acceptable excipients or carriers. In an embodiment, the composition is a pharmaceutical composition comprising a pharmaceutical effective amount of one or more active ingredients; and one or more pharmaceutically acceptable excipients or carriers. The term “pharmaceutical composition” refers to a composition suitable for use in the pharmaceutical technology with medical use. The term “therapeutically effective amount of an active ingredient” as used herein, refers to the amount of a pharmaceutical active ingredient that, when administered, is sufficient to prevent development of, or alleviate to some extent, one or more of the symptoms of the disease which is addressed. The dose of the pharmaceutically active ingredient administered will of course be determined by the particular circumstances surrounding the case, including the compound administered, the route of administration, the particular condition being treated, and the similar considerations. The pharmaceutical compositions of the present invention comprise one or more pharmaceutically acceptable excipients or carriers. The term “pharmaceutically acceptable excipients or carriers” refers to that excipients or carriers suitable for use in the pharmaceutical technology for preparing compositions with medical use. In an embodiment, the compositions of the present invention comprise one or more pharmaceutically acceptable excipients and/or carriers selected from the group consisting of diluent, binder, glidant, disintegrant, lubricant and mixtures thereof. Additionally, the pharmaceutical compositions of the present invention may contain other ingredients, such as fragrances, colorants, and other components known in the state of the art.

In an embodiment, the composition is a diagnostic composition comprising a diagnostically effective amount of one or more detection (diagnostic) agents; and one or more diagnostically acceptable excipients of carriers. The term “diagnostic composition” refers to a composition suitable for use in diagnostic, particularly in imaging diagnostic technology. The term “diagnostically effective amount of a detection moiety” as used herein, refers to the effective amount of a detection compound that, when administered, is sufficient for the diagnosis of a disease or disorder; particularly as imaging diagnostic use as contrast imaging agent. The dose of the detection compound administered will of course be determined by the particular circumstances surrounding the case, including the compound administered, the route of administration, the particular condition being diagnosticated, and the similar considerations. The diagnostic composition of the present invention comprise one or more diagnostically acceptable excipients or carriers. The term “diagnostically acceptable” refers to that excipients or carriers suitable for use in the diagnosing technology for preparing compositions with diagnostic use; particularly by imaging diagnostic use. The detection of these diagnostic agents in the body of the patient can be carried out by the well-known techniques used such as in imaging diagnostic with magnetic resonance imaging (MRI) and X-ray.

In an embodiment, the composition is a cosmetic composition comprising a cosmetically effective amount of one or more cosmetic active ingredients; and one or more cosmetically acceptable excipients of carriers. The term “cosmetic composition” refers to a composition suitable for use in cosmetic for the body care. The term “cosmetically effective amount” as used herein, refers to the effective amount of a cosmetic active agent that, when administered, is intended to improve its appearance or to beautify, preserve, condition, cleanse, color or protect the skin, nails or hair without non-medical application. The dose of the cosmetic active ingredient administered will of course be determined by the particular circumstances surrounding the case, including the compound administered, the route of administration, the particular condition and the similar considerations. The cosmetic composition of the present invention comprise one or more cosmetically acceptable excipients or carriers. The term “cosmetically acceptable” which is herein refers to that excipients or carriers suitable for use in contact with human skin, nail or hair without undue toxicity, incompatibility, instability, allergic response, among others.

The composition can be in form of topical composition, oral composition, and injectable composition.

In an embodiment, the composition is an oral composition; particularly selected form liquid or solid oral composition. In an embodiment, the composition of the invention is a solid oral composition. The oral solid compositions of the invention can be formulated in any form that includes any single unit dosage form and any multiple unit dosage forms. The term “single unit” encompasses one entity such as a single tablet, a single granule, and a single pellet. The term “single unit dosage form” defines a dosage form which consists only of one unit which contains the effective amount of the aminosquaramide polymer of the present invention. The term “multiple unit dosage form” defines a dosage from which consists of more than one unit which contains the effective amount of aminosquaramide polymer of the present invention. Usually, the multiple unit dosage forms are based on subunits such as granules, pellets or minitablets. They are usually delivered in hard gelatine capsules or transformed into tablets. Thus, it is also part of the invention a unit dosage from which comprises the composition of the present invention. In an embodiment, the unit dosage from which comprises the composition of the present invention is a single unit dosage form. In an embodiment, the unit dosage from which comprises the composition of the present invention is a multiple unit dosage form.

In an embodiment, the composition is a topical composition. The topical compositions of the invention can be formulated in several forms that include, but are not limited to, solutions, aerosols, and non-aerosol sprays, shaving creams, powders, mousses, lotions, gels, sticks, ointments, pastes, creams, shampoos, shower gel, body washes or face washes.

In an embodiment, the composition is an injectable composition; particularly selected from the group consisting of intramuscular, subcutaneous, or intravenous application. In an embodiment, the compositions of the present invention are in form of parenteral compositions suitable for their injection, infusion, or implantation into the body. The parenteral compositions defined above should be sterile, and pyrogen-free, and they can be in form of liquid such as solutions, emulsions, or suspensions, or in solid form packaged in either single-dose or multidose containers suitably diluted before use. Parenteral compositions can comprise appropriate excipients or carriers for parenteral administration that can be pharmaceutical or cosmetic excipients, including, but not limited to, solvents, suspending agents, buffering agents, substances to make the preparation isotonic with blood, stabilizers, or antimicrobial preservatives. The addition of excipients should be kept to a minimum. When excipients are used, they should not adversely affect the stability, bioavailability, safety, or efficacy of the components, or cause toxicity or undue local irritation. There should not be any incompatibility between any of the components of the dosage form.

The appropriate form of the composition can be readily determinate by those skilled in the art according to its intended use. Furthermore, the excipients and/or carriers, and their amounts, can readily be determined by those skilled in the art according to the type of formulation being prepared.

It is also a part of the invention a kit comprising an aminosquaramide polymer of the first aspect of the invention, or alternatively a conjugate of the second aspect of the invention; or alternatively a composition of the third aspect of the invention; and optionally means for its use.

In an embodiment, the kit of the present invention comprises:
An aminosquaramide polymer of the first aspect of the invention or a composition containing the aminosquaramide polymer;
optionally means to prepare the conjugate; and
optionally means to administrate the conjugate.

In an embodiment, the kit of the present invention comprises: a conjugate of the second aspect of the invention or a composition containing the conjugate; and optionally means to administrate the conjugate.

Examples of appropriate means for the preparation of the conjugate include the one or more molecules of interest, reagents for its preparation such as for example solvents, and instructions for its preparation. Examples of appropriate means for their administration include reagents and/or solvents for its use, as well as equipment (such as syringe) and instructions for its use.

As it is mentioned above, the aminosquaramide polymers of the present invention are suitable carriers for including active moieties and/or for conjugating them. Therefore, the fourth aspect of the invention refers to the use of an aminosquaramide polymer wherein Z and Z′ are inert moieties as carrier; or alternatively a composition containing it.

The fifth aspect of the invention refers to an aminosquaramide polymer selected from the group consisting of an aminosquaramide polymer comprising a backbone of repeating structural units, being these repeating structural units equal or different and selected from the group consisting of monomer of formula A or a salt thereof as defined herein above and below, and monomer of formula B or a salt thereof as defined above and below, wherein:
X is selected from the group consisting of —(CH₂)_{q}—, —(CHR₁)_{q}— and —(CR₁R₂)_{q}—;
Y is selected from the group consisting of —(CH₂)ᵣ—, —(CHR₃)ᵣ— and —(CR₃R₄)ᵣ—;
R₁ and R₂ are independently selected from the group consisting of H and Z′;
R₃ and R₄ are independently selected from the group consisting of H and Z′;
each Z and Z′ are independently selected from the group consisting of an inert moiety, a detection moiety, and an active moiety;
the inert moiety is selected from the group consisting of —(C₁-C₁₂)alkyl; —(C₂-C₁₂)alkenyl; —(C₂-C₁₂)alkylene-O—(Y—O)_{w}—(C₁-C₁₂)alquil, —(C₂-C₁₂)alkylene-O—(Y—O)_{w}—(C₂-C₁₂)alkylene-NR₁₁R₁₃, —(C₂-C₆)alkynyl; -Cy1; —(C₁-C₁₂)alkylene-Cy1; —(C₂-C₁₂)alkenylene-Cy1; —(C₂-C₁₂)alkynylene-Cy1; -Cy2-(C₁-C₁₂)alkyl; -Cy2-(C₂-C₁₂)alkenyl; and -Cy2-(C₂-C₁₂)alkynyl; being Cy1 a known ring system independently selected from the group consisting of 5- to 7-membered saturated or partially unsaturated carbocyclic or heterocyclic ring; and 5- to 7-membered carbocyclic or heterocyclic aromatic ring; Cy2 a bivalent known ring system independently selected from the group consisting of 5- to 7-membered saturated or partially unsaturated carbocyclic or heterocyclic ring; and 5- to 7-membered carbocyclic or heterocyclic aromatic ring; and being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl; being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl;
R₅ and R₆ are independently selected from the group consisting of —(C₁-C₁₂)alkyl and an amine protecting group;
R₁₁ and R₁₃ are independently selected from the group consisting of H, —(C₁-C₁₂)alkyl and an amine protecting group;
Halogen is selected from the group consisting of F, Cl, Br, and I;
the detection moiety is selected from the group consisting of chromophore moiety, fluorescent moiety, phosphorescent moiety, luminescent moiety, light absorbing moiety, radioactive moiety, transition metal and isotope mass tag moiety; and
the active moiety is selected from the group consisting of a pharmaceutically active ingredient, a veterinary active ingredient, and a cosmetic active ingredient;
p is an integer from 1 to 10;
q is an integer from 2 to 6;
r is an integer from 2 to 4;
the percentage by weight of the monomer of formula A or a salt thereof is from 30% to 100% by weight;
the percentage by weight of the monomer of formula B or a salt thereof is from 0% to 70% by weight;
being the sum of the percentages equal to 100%; and
the aminosquaramide polymer has a weight average molecular weight (Mw) from 2000 to 91000 Da;
an aminosquaramide polymer comprising repeating structural units equal or different of formula (II) or a salt thereof as defined herein above and below wherein: m is 0 or 1; and n is an integer from 10 to 400;
an aminosquaramide polymer comprising repeating structural units equal or different of formula (II) or a salt thereof as defined herein above and below wherein: m is 0 or 1; and n is an integer from 20 to 180; and
an aminosquaramide polymer as defined in the first aspect of the invention;
wherein at least one of Z and Z′ is a pharmaceutically or veterinary active moiety for use in therapy;
or alternatively, a conjugate as defined herein above and below containing the aminosquaramide polymer wherein at least one of Z and Z′ is a pharmaceutically or veterinary active moiety for use in therapy;
or alternatively, a composition as defined herein above and below containing the aminosquaramide polymer wherein at least one of Z and Z′ is a pharmaceutically or veterinary active moiety for use in therapy;
or alternatively, a composition as defined herein above and below containing the conjugate as defined herein above and below containing the aminosquaramide polymer wherein at least one of Z and Z′ is a pharmaceutically or veterinary active moiety for use in therapy;
or alternatively, a conjugate as defined herein above and below wherein the molecule of interest is at least a molecule selected from the group consisting of a pharmaceutically or veterinary active ingredient, an amino acid-containing compound, a nucleic acid-containing compound, or a mixture thereof, for use in therapy;
or alternatively, a composition as defined herein above and below containing the conjugate as defined herein above and below wherein the molecule of interest is at least a molecule selected from the group consisting of a pharmaceutically or veterinary active ingredient, an amino acid-containing compound, a nucleic acid-containing compound, or a mixture thereof, for use in therapy;
or alternatively, a conjugate as defined herein above and below containing the aminosquaramide polymer wherein at least one of Z and Z′ is a pharmaceutically or veterinary active moiety and the molecule of interest is at least a molecule selected from the group consisting of a pharmaceutically or veterinary active ingredient, an amino acid-containing compound, a nucleic acid-containing compound, or a mixture thereof, for use in therapy; and
or alternatively, a composition as defined herein above and below containing the conjugate as defined herein above and below containing an aminosquaramide polymer wherein at least one of Z and Z′ is a pharmaceutically or veterinary active moiety and the molecule of interest is at least a molecule selected from the group consisting of a pharmaceutically or veterinary active ingredient, an amino acid-containing compound, a nucleic acid-containing compound, or a mixture thereof, for use in therapy.

The use in therapy can be also drafted as a method for the prophylaxis and/or treatment of a disease which comprises administering to mammals in need of such treatment an effective amount of the aminosquaramide polymer or the conjugate disclosed herein above in the fifth aspect of the invention, together with one or more appropriate pharmaceutically acceptable excipients or carriers. Further, the use in therapy can be also reformulated as the use of the aminosquaramide polymer as defined herein above and below or the conjugate disclosed herein above and below in the fifth aspect of the invention for the preparation of a medicament for the prophylaxis and/or treatment of a disease or condition. The type of disease or condition to be treated depend on the pharmacological activity of the active ingredient of the polymer and/or the molecule of interest of the molecule.

The sixth aspect of the invention refers to:

An aminosquaramide polymer selected from the group consisting of an aminosquaramide polymer comprising a backbone of repeating structural units, being these repeating structural units equal or different and selected from the group consisting of monomer of formula A or a salt thereof as defined herein above and below, and monomer of formula B or a salt thereof as defined above and below, wherein:
X is selected from the group consisting of —(CH₂)_{q}—, —(CHR₁)_{q}— and —(CR₁R₂)_{q}—;
Y is selected from the group consisting of —(CH₂)ᵣ—, —(CHR₃)ᵣ— and —(CR₃R₄)ᵣ—;
R₁ and R₂ are independently selected from the group consisting of H and Z′;
R₃ and R₄ are independently selected from the group consisting of H and Z′;
each Z and Z′ are independently selected from the group consisting of an inert moiety, a detection moiety, and
an active moiety;
the inert moiety is selected from the group consisting of —(C₁-C₁₂)alkyl; —(C₂-C₁₂)alkenyl; —(C₂-C₁₂)alkylene-O—(Y—O)_{w}—(C₁-C₁₂)alquil, —(C₂-C₁₂)alkylene-O—(Y—O)_{w}—(C₂-C₁₂)alkylene-NR₁₁R₁₃, —(C₂-C₆)alkynyl; -Cy1; —(C₁-C₁₂)alkylene-Cy1; —(C₂-C₁₂)alkenylene-Cy1; —(C₂-C₁₂)alkynylene-Cy1; -Cy2-(C₁-C₁₂)alkyl; -Cy2-(C₂-C₁₂)alkenyl; and -Cy2-(C₂-C₁₂)alkynyl; being Cy1 a known ring system independently selected from the group consisting of 5- to 7-membered saturated or partially unsaturated carbocyclic or heterocyclic ring; and 5- to 7-membered carbocyclic or heterocyclic aromatic ring; Cy2 a bivalent known ring system independently selected from the group consisting of 5- to 7-membered saturated or partially unsaturated carbocyclic or heterocyclic ring; and 5- to 7-membered carbocyclic or heterocyclic aromatic ring; and being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl; being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl;
R₅ and R₆ are independently selected from the group consisting of —(C₁-C₁₂)alkyl and an amine protecting group;
R₁₁ and R₁₃ are independently selected from the group consisting of H, —(C₁-C₁₂)alkyl and an amine protecting group;
Halogen is selected from the group consisting of F, Cl, Br, and I;
the detection moiety is selected from the group consisting of chromophore moiety, fluorescent moiety, phosphorescent moiety, luminescent moiety, light absorbing moiety, radioactive moiety, transition metal and isotope mass tag moiety; and
the active moiety is selected from the group consisting of a pharmaceutically active ingredient, a veterinary active ingredient, and a cosmetic active ingredient;
p is an integer from 1 to 10;
q is an integer from 2 to 6;
r is an integer from 2 to 4;
the percentage by weight of the monomer of formula A or a salt thereof is from 30% to 100% by weight;
the percentage by weight of the monomer of formula B or a salt thereof is from 0% to 70% by weight;
being the sum of the percentages equal to 100%; and
the aminosquaramide polymer has a weight average molecular weight (Mw) from 2000 to 91000 Da;
an aminosquaramide polymer comprising repeating structural units equal or different of formula (II) or a salt thereof as defined herein above and below wherein: m is 0 or 1; and n is an integer from 10 to 400;
an aminosquaramide polymer comprising repeating structural units equal or different of formula (II) or a salt thereof as defined herein above and below wherein: m is 0 or 1; and n is an integer from 20 to 180; and
an aminosquaramide polymer as defined in the first aspect of the invention;
wherein at least one of Z and Z′ is a detection moiety for use in diagnostic;
or alternatively, a conjugate as defined herein above and below containing the aminosquaramide polymer wherein at least one of Z and Z′ is a detection moiety for use in diagnostic; and
or alternatively, a composition as defined herein above and below containing the aminosquaramide polymer wherein at least one of Z and Z′ is a detection moiety for use in diagnostic.

The seventh aspect of the invention refers to:
a conjugate as defined herein above and below wherein the molecule of interest is at least a nucleic acid-containing compound for use as a transfecting agent;
or alternatively a composition as defined herein above and below containing the conjugate wherein the molecule of interest is at least a nucleic acid-containing compound for use as a transfecting agent.

The eighth aspect of the invention refers to the use in cosmetic of:
an aminosquaramide polymer selected from the group consisting of an aminosquaramide polymer comprising a backbone of repeating structural units, being these repeating structural units equal or different and selected from the group consisting of monomer of formula A or a salt thereof as defined herein above and below, and monomer of formula B or a salt thereof as defined above and below,
wherein:
X is selected from the group consisting of —(CH₂)_{q}—, —(CHR₁)_{q}— and —(CR₁R₂)_{q}—;
Y is selected from the group consisting of —(CH₂)ᵣ—, —(CHR₃)ᵣ— and —(CR₃R₄)ᵣ—;
R₁ and R₂ are independently selected from the group consisting of H and Z′;
R₃ and R₄ are independently selected from the group consisting of H and Z′;
each Z and Z′ are independently selected from the group consisting of an inert moiety, a detection moiety, and an active moiety;
the inert moiety is selected from the group consisting of —(C₁-C₁₂)alkyl; —(C₂-C₁₂)alkenyl; —(C₂-C₁₂)alkylene-O—(Y—O)_{w}—(C₁-C₁₂)alquil, —(C₂-C₁₂)alkylene-O—(Y—O)_{w}—(C₂-C₁₂)alkylene-NR₁₁R₁₃, —(C₂-C₆)alkynyl; -Cy1; —(C₁-C₁₂)alkylene-Cy1; —(C₂-C₁₂)alkenylene-Cy1; —(C₂-C₁₂)alkynylene-Cy1; -Cy2-(C₁-C₁₂)alkyl; -Cy2-(C₂-C₁₂)alkenyl; and -Cy2-(C₂-C₁₂)alkynyl; being Cy1 a known ring system independently selected from the group consisting of 5- to 7-membered saturated or partially unsaturated carbocyclic or heterocyclic ring; and 5- to 7-membered carbocyclic or heterocyclic aromatic ring; Cy2 a bivalent known ring system independently selected from the group consisting of 5- to 7-membered saturated or partially unsaturated carbocyclic or heterocyclic ring; and 5- to 7-membered carbocyclic or heterocyclic aromatic ring; and being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl; being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl;
R₅ and R₆ are independently selected from the group consisting of —(C₁-C₁₂)alkyl and an amine protecting group;
R₁₁ and R₁₃ are independently selected from the group consisting of H, —(C₁-C₁₂)alkyl and an amine protecting group;
Halogen is selected from the group consisting of F, Cl, Br and I;
the detection moiety is selected from the group consisting of chromophore moiety, fluorescent moiety, phosphorescent moiety, luminescent moiety, light absorbing moiety, radioactive moiety, transition metal and isotope mass tag moiety; and
the active moiety is selected from the group consisting of a pharmaceutically active ingredient, a veterinary active ingredient and a cosmetic active ingredient;
p is an integer from 1 to 10;
q is an integer from 2 to 6;
r is an integer from 2 to 4;
the percentage by weight of the monomer of formula A or a salt thereof is from 30% to 100% by weight;
the percentage by weight of the monomer of formula B or a salt thereof is from 0% to 70% by weight;
being the sum of the percentages equal to 100%; and
the aminosquaramide polymer has a weight average molecular weight (Mw) from 2000 to 91000 Da;
an aminosquaramide polymer comprising repeating structural units equal or different of formula (II) or a salt thereof as defined herein above and below wherein: m is 0 or 1; and n is an integer from 10 to 400;
an aminosquaramide polymer comprising repeating structural units equal or different of formula (II) or a salt thereof as defined herein above and below wherein: m is 0 or 1; and n is an integer from 20 to 180; and an aminosquaramide polymer as defined in the first aspect of the invention;
wherein at least one of Z and Z′ is a cosmetically active moiety;
or alternatively, a conjugate as defined herein above and below containing the aminosquaramide polymer wherein at least one of Z and Z′ is a cosmetically active moiety;
or alternatively, a composition as defined herein above and below containing the aminosquaramide polymer wherein at least one of Z and Z′ is a cosmetically active moiety;
or alternatively, a composition as defined herein above and below containing the conjugate containing the aminosquaramide polymer wherein at least one of Z and Z′ is a cosmetically active moiety;
or alternatively, a conjugate as defined herein above and below wherein the molecule of interest is at least one cosmetically active ingredient;
or alternatively, a composition as defined herein above and below containing the conjugate wherein the molecule of interest is at least one cosmetically active ingredient;
or alternatively, a conjugate as defined herein above and below containing the aminosquaramide polymer wherein at least one of Z and Z′ is a cosmetically active moiety and the molecule of interest is at least one cosmetically active ingredient;
or alternatively, a composition as defined herein above and below containing the conjugate containing an aminosquaramide polymer wherein at least one of Z and Z′ is a cosmetically active moiety and the molecule of interest is at least one cosmetically active ingredient.

All disclosed above for the aminosquaramide polymer, conjugate and composition, particularly in relation to the active ingredients and the molecules of interest also apply here for their uses in therapy, in diagnostic, in cosmetic and as transfecting agent.

Throughout the description and claims, the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description, or may be learned by practice of the invention. The following examples and drawings are provided by way of illustration, and they are not intended to be limiting of the present invention. Reference signs related to drawings and placed in parentheses in a claim, are solely for attempting to increase the intelligibility of the claim, and shall not be construed as limiting the scope of the claim. Furthermore, the present invention covers all possible combinations of particular and preferred embodiments described herein.

### EXAMPLES

### Abbreviations

aa: Amino acid
A.R grade: grade of analytical reagent
BOC: tert-butoxycarbonyl protecting group
CAR: Chimeric antigen receptor
CPPs: Cell penetrating peptides
CRISPR: Clustered regularly interspaced short palindromic repeats.
Cas9: protein 9 nuclease
CRISPR/cas9: Clustered regularly interspaced short palindromic repeats associated system with nuclease protein 9
decoy DNA: DNA molecular decoy
DES: Diethyl squarate
DNA: Deoxyribonucleic acid
DOSY: Diffusion-Ordered Spectroscopy
EGA: End group analysis
FBS: Fetal Bovine Serum
GFP: Green fluorescent protein
GPC: Gel Permeation Chromatography
GPC-HRMS: Gel Permeation Chromatography-High Resolution Mass Spectra
GPC-UV: Gel Permeation Chromatography-Visible Ultraviolet
gRNA: Guide ribonucleic acid
HEK293: Human embryonic kidney cells
Hela: Human cervix cells
HMW: High molecular weight
LMW: low molecular weight
LNA: Locked nucleic acid
LNPs: Lipid nanoparticles
MA104: Fetal African green monkey kidney epithelial cells
MEM: Minimum Essential Medium
miRNA: Micro ribonucleic acid
MMW: Medium molecular weight
Mn: Number Average Molecular weight
Mw: Weight Average Molecular weight
mNeonGreen gene: Gene that encodes a Green fluorescent protein
MOPS: 3-(N-Morpholino)propane sulfonic acid
mScarlet gene: Gene that encodes a red fluorescent protein
MW: Molecular weight
Neo: Neomycin phosphotransferase
NIH3T3: Non-transformed murine fibroblasts
NMR: Nuclear Magnetic Resonance
pASQUA: Polyaminosquaramide
PC-3: Metastatic prostate cancer (human transformed cells)
PCR: Polymerase chain reaction
pDsRed-Express-C1: Vector for fusing DsRed-Express to the N-terminus of a partner protein
pESQUA: Polioxoethylenesguaramide
pMAX-GFP: plasmid that encodes green fluorescent protein
PNA: Peptide nucleic acid
pTREX-Ngfp: plasmid of 7.1 kb that encodes a Green fluorescent protein in T. cruzi.
RifR primers: Rule Interchange Format
RNA: Ribonucleic acid
RPMI: Roswell Park Memorial Institute medium
sgRNA: Single guide ribonucleic acid
shRNA: Short hairpin ribonucleic acid
siRNA: Small Interfering ribonucleic acid
SW480: Colorectal cancer (human transformed cells)
TCTs: Tissue culture TcCLBr-RedScarlettrypomastigotes
VERO: African green monkey kidney cells
XP2YO: Xeroderma pigmentosum human cell line)

### Material and Methods

Chemicals and solvents are either A.R grade or purified by standard techniques. Diethyl squarate (DES-(VII)) and 3,3′-diamino-N-methyldipropylamine were vacuum distilled just prior their use. Cell lines, primers, nucleic acids, protein nucleic acids, enzymes and plasmids used in the present application are commercially available. Protozoa are freely available. Non-transformed murine fibroblasts (NIH3T3), human embryonic kidney cells (HEK293), and human cancer cell lines (PC-3, metastatic prostate cancer; SW480, colorectal cancer) are available from ATCC (https://www.lgcstandards-atcc.org/en/About/About_ATCC/Who_We_Are.aspx). And XP2YO (xeroderma pigmentosum cells) are available from Coriell Institute for Medical Research (https://www.coriell.org/). Gel Permeation Chromatography (GPC) analysis was performed using a HPLC system from Gilson composed of a liquid handler module 215, a pump module 322-H2 and a diode-array detector 172. Two GPC columns were run in series (Agilent PL Aquagel-OH 20, 5 μm, 300×7.5 mm and Aquagel-OH 40, 8 μm, 300×7.5 mm) with a flow rate 0.5 mL/min and H₂O containing HCOOH (0.1% v/v), pH=2.7-2.8 as the mobile phase.

Nuclear Magnetic Resonance (NMR) for chemical characterization ¹H and ¹³C NMR were performed with a Bruker Avance 600 (600 MHz) spectrometer. The chemical shifts are expressed in δ relative to DSS (δ=0 ppm). The spectra were recorded in H₂O (containing 0.1% formic acid) using 5 mm tubes with a coaxial tube containing D₂O for lock and sodium 2,2-dimethyl-2-silapentane-5-sulfonate (DSS) as internal reference. Water suppression was performed using a zggpw5 pulse sequence.

Determination of the Weight average molecular weight (Mw) and the Number Average Molecular weight (Mn) of the aminosquaramide compounds of the present invention and its fractions were independently assessed by, GPC-UV/GPC-HRMS, 2D NMR (DOSY), and end group analysis (EGA). Gel Permeation Chromatography—High Resolution Mass Spectra (GPC-HRMS), was performed using two Aquagel-OH columns, as described above, running on a UHPLC (Ultimate 3000, Thermo Fisher Scientific) system coupled to a Q-Exactive Hybrid Quadrupole-Orbitrap (Thermo Fisher Scientific) operating with a heated electrospray interface (HESI), the spectra have been recorded positive. This technique allowed the identification of oligomers with the same structural composition of the aminosquaramide compounds of the present invention falling outside the scope of the present invention. After digital filtering, the retention times measured for these oligomers, together with those recorded by GPC-UV for pure samples of isolated oligomers, were used to construct a calibration curve for GPC analysis of the aminosquaramide compounds of the present invention fractions. This method also provided the polydispersity index values.

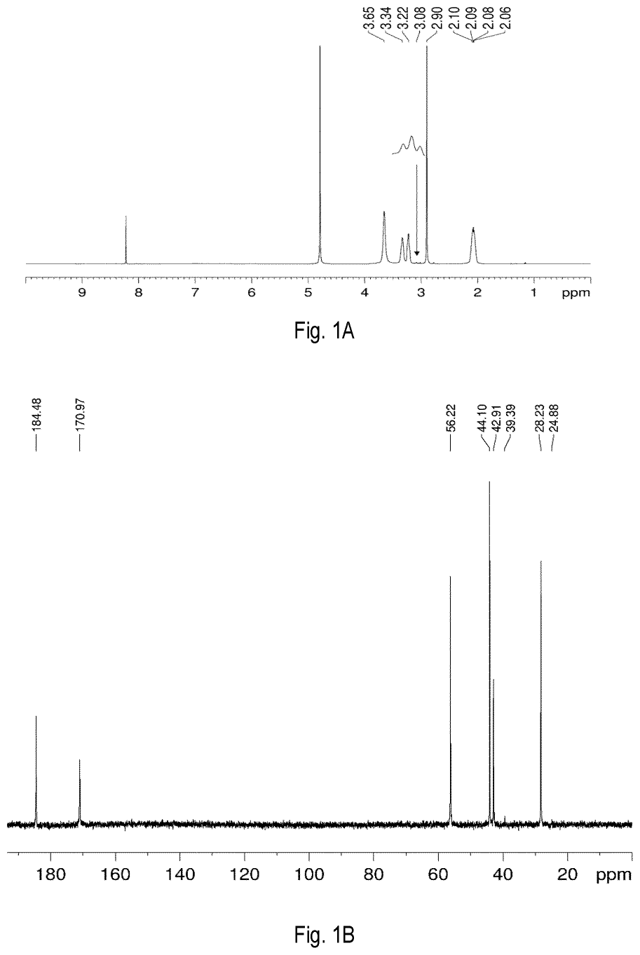

The 2D NMR spectra were recorded on a Bruker Avance 600 MHz. The NMR samples were equilibrated at 298 K for 10 min before data collection. DOSY was performed on samples of the aminosquaramide compound of the invention in D₂O-MeOD (5% v/v) containing 0.1% v/v HCOOH. Each tube was filled with 5 mg of aminosquaramide compound in 0.6 mL of solvent. The spectra were acquired using a ledbpgp2s pulse sequence. Normalized diffusion coefficients were obtained using 2,6-diamino pyridine as the internal reference (cf. Neufeld, R. et al. “Accurate Molecular Weight Determination of Small Molecules via DOSY-NMR by Using External Calibration Curves with Normalized Diffusion Coefficients”. Chem. Sci. 2015, vol. 6 (6), pp. 3354-3364). To further evaluate the molecular weight range of aminosquaramide compounds of the invention, Mn values were evaluated by end group analysis (EGA) by accurate integration of the NMR triplet at 3.09 ppm assigned to a methylene group located next to a terminal amine group (cf. FIG. 1). The values disclosed hereinbelow were obtained by assuming the formation of linear polymeric chains containing on average one terminal amino methylene residue.

### 1. Aminosquaramide Polymers (I) of the Present Invention

### 1.1. Mixture 1 of Compounds of Formula (I)-1A-1 and (I)-1B-1

### 1.1.1. Composition

The mixture 1 of the present invention comprises aminosquaramide polymers of formula (I)-1A-1 and (I)-1B-1 wherein m is 0, X is —(CH₂)₃—, Z is CH₃, R₃ and R₄ are H, n is from 26 to 76.

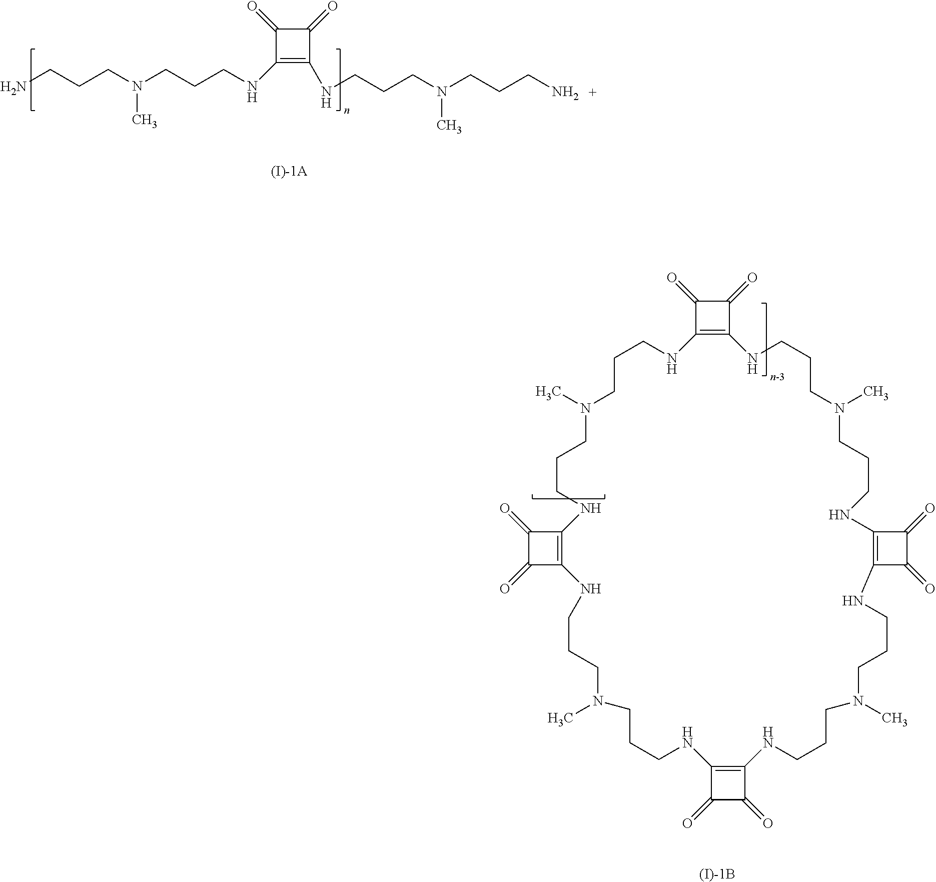

### 1.1.2. Preparation and Purification Process of Mixture 1

Mixture 1 was prepared following the methods disclosed below. In particular, method A allowed a unique fraction of mixture 1 to be obtained having a numeral average molecular weight (Mn) about 24100 Da, determined by GPC-UV. Meanwhile, methods B and C allowed two different fractions of mixture 1 to be obtained, a low molecular weight fraction (having a Mn of 7700 Da by method B, and a Mn of 13300 Da by method C determined by GPC-UV) and a high molecular weight fraction (having a Mn of 16400 Da by method B and a Mn of 20800 Da by method C determined by GPC-UV).

### Method A

Method A comprises reacting the diester (IV) and the diamine (V) to obtain crude mixture 1, which is further purified by repeating cycles of a re-dissolution/precipitation-filtration process. A scheme of the synthesis of mixture 1 prepared by method A is disclosed below:

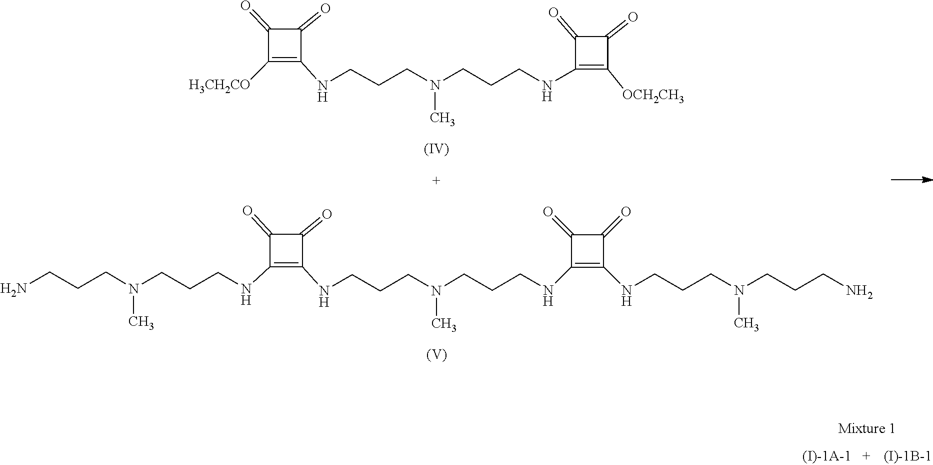

### Synthesis of Diester (IV)

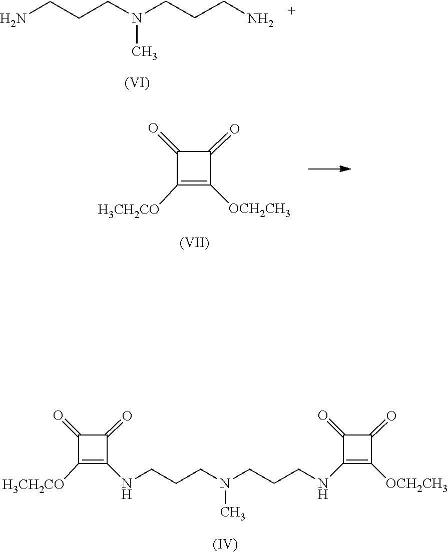

A solution of diamine (VI) (2.54 g, 17.5 mmol) in ethyl ether (235 mL) was added dropwise to a solution of DES (VII) (6.53 g, 38.4 mmol) in 140 mL of diethyl ether, and the mixture was kept under argon atmosphere for an additional 12 h, with stirring at room temperature. The resulting solid was filtered and purified by digesting a slurry of the solid with ethyl ether (3×60 mL) to yield 6.10 g (89%) of diester (IV) as a pale-yellow solid.

### Synthesis of Diamine (V)

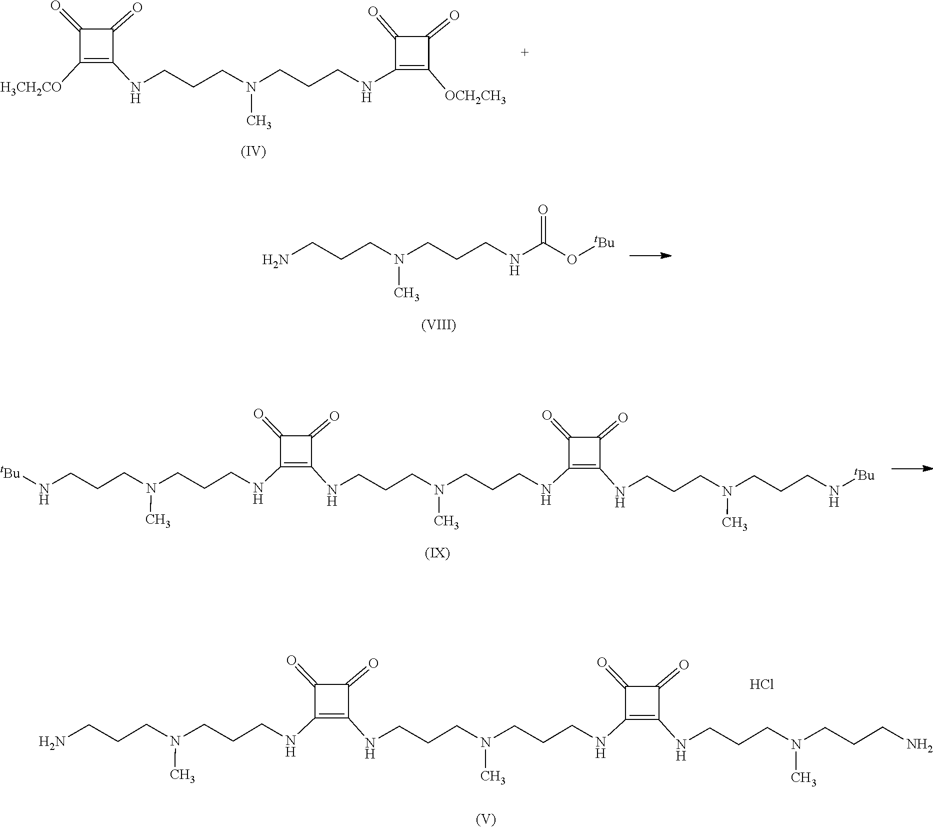

A solution of diester (IV) (2.52 g, 6.4 mmol) in 200 mL ethanol was added dropwise to a solution of tert-butyl [3-[(3-aminopropyl)-methylamino]-propyl]carbamate (VIII) (3.38 g, 13.5 mmol) in ethanol (100 mL). The reaction mixture was kept under argon atmosphere for an additional 12 h, with stirring at room temperature. The solution was concentrated to dryness and the residue was taken up in 100 mL of methylene chloride, washed with water and brine, and the solvent evaporated to afford 4.5 g (90%) of BOC protected diamine (IX) as a white solid. 14.4 mL 3 M HCl was added to a suspension of the BOC-protected diamine (IX) (2.97 g, 3.8 mmol) in water (200 mL), and the solution was kept under argon atmosphere for an additional 12 h, with stirring at 50° C. The solvent was concentrated under vacuum, affording the diamine (V) as the hydrochloride salt that was used immediately in the next step.

### Synthesis and Separation of a Low Molecular Weight Fraction and a High Molecular Weight Fraction of Mixture 1

Solid Cs₂CO₃ (5.2 g) was added to a suspension of the diamine hydrochloride (VI) (1.77 g, 2.3 mmol) in ethanol (15 mL) and the mixture was stirred for 1 h at room temperature. Then, a solution of the diester (IV) (0.90 g, 2.3 mmol) in 15 mL ethanol was added and the reaction mixture was kept under argon atmosphere and stirred for an additional 5 h. The mixture was treated with a dilute solution of HCl (50 mL, 10⁻³ M) and concentrated in a rotary evaporator (pH 6.8). The resulting aqueous solution was basified by adding solid sodium carbonate (pH 8.2) to produce crude mixture 1 (2.5 g).

This crude mixture 1 was purified by five sequential cycles of re-dissolution/precipitation-filtration at pH 3 for the re-dissolution steps and from 7.4-8.1 for the re-precipitation steps. The unique purified fraction of mixture 1 of the present invention obtained by method A has a number average molecular weight (Mn) of 24100 Da determined by GPC-UV.

### Method B

Method B comprises reacting the diester (IV) and the diamine (V) to obtain crude mixture 1, which is further purified by repeating cycles of fractionation and purification to obtain a low molecular weight fraction and a high molecular weight fraction of mixture 1. The starting materials diester (IV) and diamine (V) were prepared following the same process as disclosed for method A.

### Synthesis and Separation of a Low Molecular Weight Fraction and a High Molecular Weight Fraction of Mixture 1

Solid Cs₂CO₃ (7.4 g) was added to a suspension of the diamine hydrochloride (V), (2.91 g, 3.8 mmol) in ethanol (35 mL) and the mixture was stirred for 1 h at room temperature. A solution of the diester (IV) (1.84 g, 4.7 mmol) in 40 mL ethanol was added, and the reaction mixture was kept under argon atmosphere and stirred for an additional 48 h. The resulting solid was filtered and washed with cold ethanol (3×20 mL), cold water (3×20 mL), 30 mL acetone, and dried at room temperature under an active vacuum for 12 h, to afford crude mixture 1 (4.6 g) as a pale-yellow solid.

Fractionation and purification of a re-dissolved crude mixture 1 solution were accomplished through a series of centrifugation steps utilizing an Ortoalresa centrifuge working with Amicon Ultra 15 mL centrifugal filters of 3 and 10 kDa MW cut-offs, respectively (Millipore, MA, USA). Briefly, a solution of the above yellow solid (0.35 g) in 15 mL HCl (10⁻³ M) or formic acid (0.1% v/v) was passed through a 3 kDa filter at 3220 g for 40 min. The filtrate solution was discharged. The concentrate was diluted with the acid solution (12 mL) and spun down again. This cycle of dilution-filtration was repeated five times. Then, the isolated concentrate was collected and subsequently transferred to a centrifugal filter with 10 kDa cut off and spun down again for 40 min, repeating the cycle of dilution-filtration five more times. Once pooled and concentrated to dryness, the solid material isolated from the filtrates affords the low molecular weight fraction of mixture 1. The isolated concentrate was collected to afford the high molecular weight fraction of mixture 1.

Therefore, fractionation of crude mixture by centrifugal filtration produced two fractions (low- and high-molecular weight), indicating an approximate molecular weight distribution between 3 kDa and 10 kDa for the low molecular weight fraction, and greater than 10 kDa for the high molecular weight fraction. Purification and fractionation of crude mixtures were verified using GPC-UV and/or GPC-HRMS analysis. The values of number average molecular weight (Mn) of low- and high-molecular weight fraction of the aminosquaramide compound of the present invention obtained by method B were 7700 and 16400 Da respectively, determined by GPC-UV.

### Method C

Method C comprises reacting the diethyl squarate (VII) and the diamine (VI) to obtain crude mixture 1, which is further purified by repeating cycles of fractionation and purification as disclosed in method B, obtaining a low molecular weight fraction and a high molecular weight fraction of mixture 1. The starting materials were commercially available. A scheme of the synthesis of mixture 1 by method C is disclosed herein.

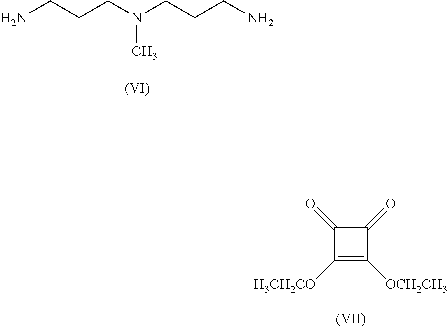

3,3′-diamino-N-methyldipropylamine (VI) (0.50 g, 3.4 mmol) in 7.5 mL ethanol was added with stirring to a solution of diethyl squarate (DES; VII) (0.58 g, 3.4 mmol) in 7.5 mL ethanol, at room temperature. A yellowish precipitate formed in seconds and stirring was continued for 4 h at room temperature. The precipitate was collected by filtration, washed with ethanol, and dried, affording 0.69 g of crude mixture 1. The crude mixture 1 was purified by centrifugal filtration following the procedure described in method B to achieve a low molecular weight fraction and a high molecular weight fraction of mixture 1. The values of number average molecular weight (Mn) of the low- and the high-molecular weight fraction of the aminosquaramide of the present invention obtained by Method C were 13300 Da and 20800 Da respectively, determined by GPC-UV.

### 1.1.3. Physico-Chemical Characterization of Mixture 1

The ¹H NMR and ¹³C NMR spectra (i.e., FIGS. 1A and 1B respectively) for the high molecular weight fraction of mixture 1 obtained by method B and isolated by centrifugal filtration were recorded.

On one hand, the ¹H and ¹³C NMR spectra showed the four relatively broad peaks assigned to the four distinct proton types (i.e., about 2.07 ppm, about 2.90 ppm, about 3.22 ppm and about 3.64 ppm) present in the aliphatic portion of the molecules forming part of mixture 1; and the four corresponding carbon resonances (i.e., 28.23 ppm, 42.91 ppm, 44.10 ppm and 56.22 ppm). On the other hand, the ¹H and ¹³C NMR spectra also showed the structural features of the inner aminosquaramide units and a diagnostic triplet at 3.09 ppm (enlarged area of the ¹H NMR spectrum), assigned to the terminal protonated amino methylene groups and a corresponding peak at 39.39 ppm in the ¹³C NMR spectrum.

### 1.1.4. Thermal and Chemical Stability of Mixture 1

The aminosquaramide compounds of formula (I) of the present invention, particularly the high molecular weight fraction of mixture 1 obtained by Method B were stable on the shelf for extended periods (more than 3 years). Upon heating, thermal decomposition occurred at temperatures higher than 230° C. Further, solutions of the high molecular weight fraction of mixture 1 obtained by method B of the present invention remained unaltered for extended periods (months) at room temperature at pH values in the range from 2-7.

For long-term storage, a solution of mixture 1 was dissolved in water (MilliQ) containing 0.1% v/v HCOOH. A measured volume of the above solution was transferred into pre-weighted 1.5 mL glass vials. Then, the vials were evaporated to dryness by an active vacuum until constant weight. Vials containing 1 to 5 mg of mixture 1 were prepared in this way. This material remains unaltered for years on the shelf.

For biological experiments, a measured volume of PBS buffer (or similar biological medium such as sodium acetate pH 5) was added to the vial and vortex-agitated for 30 s before using.

The aminosquaramide compounds of formula (I) of the present invention were then stable under the reaction, manufacturing, storage and use conditions.

### 1.1.5. Transfection Study

The aminosquaramide of the present invention are suited to deliver biologically active molecules into a wide variety of cells, including, but not limited to, cell lines commonly used in research laboratory settings, cultivable protozoan parasites, free-living protists, bacteria, and in addition, cells lines and lineages that are typically considered difficult to transfect, such as primary cell cultures. The biologically active molecules thus transfected retain activity after delivery. The active molecules that can be delivered into cells may be any biological molecule of interest such as macromolecules, including but not limited to, negatively charged macromolecules. Examples of molecules of interest include peptides and nucleic acids (i.e. DNA, RNA, PNA and other synthetic nucleic acids).

The transfecting method that uses the aminosquaramide of the present invention can facilitate the simultaneous intracellular delivery of up to about 10 nucleic acid expression sequences that encode distinct polypeptides. Or alternatively, non-coding RNA molecules such as siRNA, miRNA, or single guide RNA (sgRNA) molecules used in genome editing experiments can also be introduced. Further, with CRISPR-mediated genome editing, sgRNA/sgDNA can be transfected in combination with the relevant DNA template molecules. This method requires the formation of a conjugate (polyplex) with the aminosquaramide of the present invention and the biologically active molecule to be transfected. As it is shown below, the aminosquaramide-nucleic acid polyplexes formed rapidly, but it is recommended to allow up to 30 min for optimal packaging. These polyplexes are stable for up to 4 hours. Transfection has been achieved with packaging reactions that have been incubated for up to 5 days, however higher efficiency is obtained when the packaging reaction is added to the target cells after 30 min incubation.

### 1.1.5.1. Transfection of Mammalian Cells in Culture

Transfection of established cell lines of diverse origin, as well as primary cultures and obligate intracellular organisms (parasites) (cf. section 2.3) were performed using the aminosquaramide compound (I) of the present invention.

### Mammalian Established Lines (DNA Transfection)

Transfection of established cell lines of diverse origin were performed using the aminosquaramide compound (I) of the present invention, particularly the high molecular weight fraction of mixture 1 obtained by method B. Several widely used mammalian cell lines, including non-transformed murine fibroblasts (NIH3T3) and human embryonic kidney cells (HEK293), together with human xeroderma pigmentosum cells (XP2YO) and human cancer cell lines (PC-3, metastatic prostate cancer; SW480, colorectal cancer) have been successfully transfected.

### Transfection Protocol

A standard protocol with the aminosquaramide compound (I) of the present invention, particularly the high molecular weight fraction of mixture 1 obtained by method B was used. Noteworthy, however, to obtain the highest transfection efficiency this standard protocol was experimentally adapted for each cell line of interest.

Mammalian cells were transfected with 1 μg of pMAX-GFP (Lonza) in 12-well plates using the following standard protocol. A transfection mix was prepared by diluting 1 μg of pMAX-GFP in 125 μl Opti-MEM® (Gibco™) and incubating it together with 12-20 ppm of the above-mentioned mixture 1 of the present invention, diluted in 125 μl of Opti-MEM® for 30 min at room temperature. The resulting 250 μl transfection mix was added to a well within a 12-well plate and then 100,000 cells in antibiotic-free medium containing 10% FBS were added dropwise to a final volume of 1 ml and 2-5 ppm of the above-mentioned mixture 1. 24 h later, the medium was replaced with fresh complete medium. As a negative and positive control, in all experiments, a replicate was left untreated (without transfection mix) and another was transfected with a standard commercial reagent (Lipofectamine2000®, Invitrogen) containing the same plasmid (i.e., pMAX-GFP), following the manufacturer's instructions.

This protocol was performed in HEK293, NIH3T3, SW480, PC-3, and XP2YO cells, the optimal ppm of mixture 1 for each cell line was worked out from the range mentioned before, 4.5 ppm of mixture 1 of the present invention was used for all the cell lines, except for XP2YO cells, which were transfected with 3 ppm. Alternatively, cells were also transfected after being seeded previously, this protocol is specially advantageous to transfect during the seeding process.

### Results

Transfection efficiency and cell viability were assessed 48 h after transfection by quantifying respectively, fluorescence (resulting from expression of the plasmid encoded GFP gene) and luminescence (using the CellTiter Glo® kit, Promega, to detect viable cells) with a spectrophotometric plate reader. The CellTiter Glo® kit reacts with ATP of living (viable) cells, generating a luminescent signal that is detected with a luminescence reader. The reading is proportional to the number of viable cells in the culture. In the case of fluorescence, the values measured can be related directly to the level of translated GFP transcripts and used to estimate the number of transfected cells.

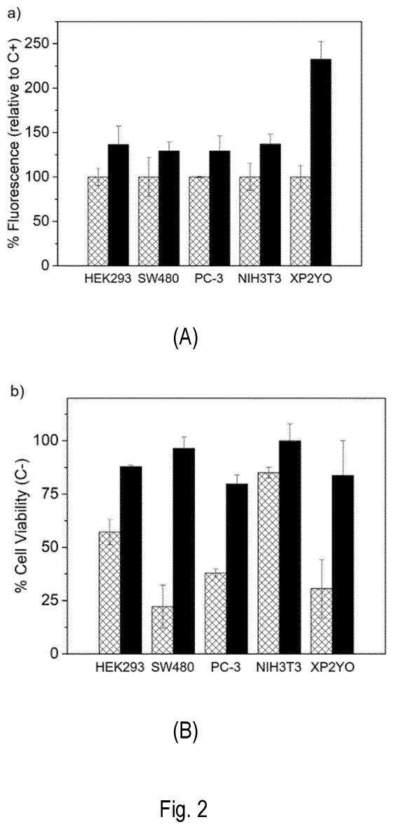

The GFP expression obtained following the transfection protocol as defined above with the aminosquaramide of the invention (mixture 1) in comparison with that obtained with Lipofectamine2000® (standard control) are shown in FIG. 2A. Cell viability data obtained with the transfection protocol with the aminosquaramide of the invention (mixture 1) or the standard positive control (Lipofectamine2000®), related to untreated non-transfected cells (negative control) are shown in FIG. 2B. These results demonstrate superior transfection efficiency, with much less toxicity (superior cell viability) using the polyaminosquaramide of the present invention in comparison with Lipofectamine2000®.

### Mammalian Primary Cultures (DNA Transfection)

Mammalian primary cells were also transfected using a direct transfection protocol. Briefly, cells were previously seeded in plates to allow attachment and development. On the day of transfection, cells were first washed with neuronal transfection buffer (90% buffered salt-glucose-glycine solution, 10% minimum essential medium (Invitrogen), insulin (7.5 μg/ml), transferrin (7.5 μg/ml), sodium selenite (7.5 ng/ml)) and then the aminosquaramide protocol, as described for the mammalian established lines with pMAX-GFP, was applied. As an example, an ex vivo rat hippocampal co-culture containing both cortical neurons and glial cells was transfected.

As with the mammalian established lines, the transfection of mammalian primary cells was more efficient than could be achieved with established methods. We observed an increase from 0.01% (Lipofectamine2000) to 8-13% (the aminosquaramide of the invention; mixture 1) in the percentage of cells transfected.

### Mammalian Established Lines (RNA Transfection)

Transfection of the HEK293T cell line was performed using the aminosquaramide compound (I) of the present invention, particularly the high molecular weight fraction of mixture 1 obtained by method B.

The transfection protocol used in the present application is Transfection protocol A.

### Transfection Protocol A

A standard protocol with the aminosquaramide compound (I) of the present invention, particularly the high molecular weight fraction of mixture 1 obtained by method B was used. A 2.6 Kb mRNA was synthesized using the in vitro transcription method provided by the mMESSAGE mMACHINE™ T7 ULTRA Transcription Kit (Invitrogen) using as a template a PCR product containing the regulatory elements required by the kit in addition to the gene of interest (Luc: Neon) which encodes a fusion protein with luciferase activity and green fluorescence.

Mammalian cells were transfected with 0.2 μg of mRNA in 12-well plates using the following standard protocol. A transfection mix was prepared diluting 0.2 μg of mRNA in 250 μl DMEM® (Gibco™) and incubating it together with 10 ppm of the above-mentioned mixture 1 of the present invention for 30 min at room temperature. The resulting 250 μl transfection mix was added to a well within a 12-well plate and then 100,000 cells in antibiotic-free medium containing 10% FBS were added dropwise up to a final volume of 1 ml. 18 h later, cells were visualized under epifluorescence microscopy. As a negative and positive control, in all experiments, a replicate was left untreated (without transfection mix) and another was transfected with a standard commercial reagent (Lipofectamine2000®, Invitrogen) including the same mRNA of the test and following the manufacturer's instructions.

For the purpose of the present invention, the alternative transfection protocol B may also be used.

### Transfection Protocol B

A standard protocol with the aminosquaramide compound (I) of the present invention, particularly the high molecular weight fraction of mixture 1 obtained by method B was used. A 2.6 Kb mRNA was synthesized using the in vitro transcription method provided by the mMESSAGE mMACHINE™ T7 ULTRA Transcription Kit (Invitrogen) using as a template a PCR product containing the regulatory elements required by the kit in addition to the gene of interest (Luc::Neon) which encodes a fusion protein with luciferase activity and green fluorescence.

Mammalian cells were transfected with 0.2 μg of mRNA in 12-well plates using the following standard protocol. A transfection mix was prepared diluting 0.2 μg of mRNA in 250 μl DMEM® (Gibco™) and incubating it together with 2-10 ppm of the above-mentioned mixture 1 of the present invention for 30 min at room temperature. The resulting 250 μl transfection mix was added to a well within a 12-well plate containing 100,000 cells (seeded from the day before) in antibiotic-free medium containing 10% FBS, after 1 h incubation final volume was topped up to 1 ml of complete medium containing 10% FBS. 18 h later, cells were visualized under epifluorescence microscopy. As a negative and positive control, in all experiments, a replicate was left untreated (without transfection mix) and another was transfected with a standard commercial reagent (Lipofectamine2000®, Invitrogen) including the same mRNA of the test and following the manufacturer's instructions.

### 1.1.5.2. Transfection of Unicellular Organisms

Transfection of Extracellular Forms of Protozoan Parasites: Trypanosoma cruzi

The transfection of replicative extracellular forms (epimastigotes) of the protozoan parasite with exogenous DNA was performed.

As an example, the transfection of replicative extracellular forms (epimastigotes) of the protozoan parasite Trypanosoma cruzi (CL Brener CLBr strain) was achieved with exogenous DNA as defined below. Any Trypanosoma cruzi strain prepared according to the state of the art can also be used. These forms of the parasite are motile and grow in suspension.

### Protozoan Parasite Vector

T. cruzi epimastigotes were cultured in supplemented RPMI-1640 as described previously (with a 0.5% (w/v) trypticase (BBL), 0.5% (w/v) HEPES, 0.03 M hemin and 10% (v/v) fetal calf serum (heat-inactivated)). They were maintained and propagated in non-vented T25 culture flasks at 1×10⁵ to 1×10⁷ epimastigotes/mL in preparation for transfection, parasites were washed twice with PBS. The plasmid pTREXn-GFP was prepared by miniprep and quantified by nanodrop.

pTREXn-GFP: Aminosquaramide Compound (Mixture 1) Complex

The complex formed by pTREXn-GFP and aminosquaramide compound (mixture 1) was prepared as follows: Packaging reactions of 500 μL were prepared in Eppendorf tubes. These contained RPMI-1640 (Sigma-Aldrich) as the diluent and contained 0.1 or 0.5 μg of plasmid DNA and 12-18 ppm of mixture 1 of the invention. The diluted preparations were mixed thoroughly and incubated at room temperature for 30 min to allow complex formation.

### Transfection Protocol

For transfection, 10⁷ parasites were pelleted, resuspended with the packaging reaction, and incubated for 1-2 h at 28° C. Then, the mixture was transferred to a culture flask containing 9.5 mL complete medium. Assessment of transfection was performed by flow cytometry 48 h later, by quantifying GFP expression. For comparison, parasites were transfected by electroporation (physical transfection method) using the Amaxa Nucleofector (program X-014), with the same amounts of DNA.

### Results

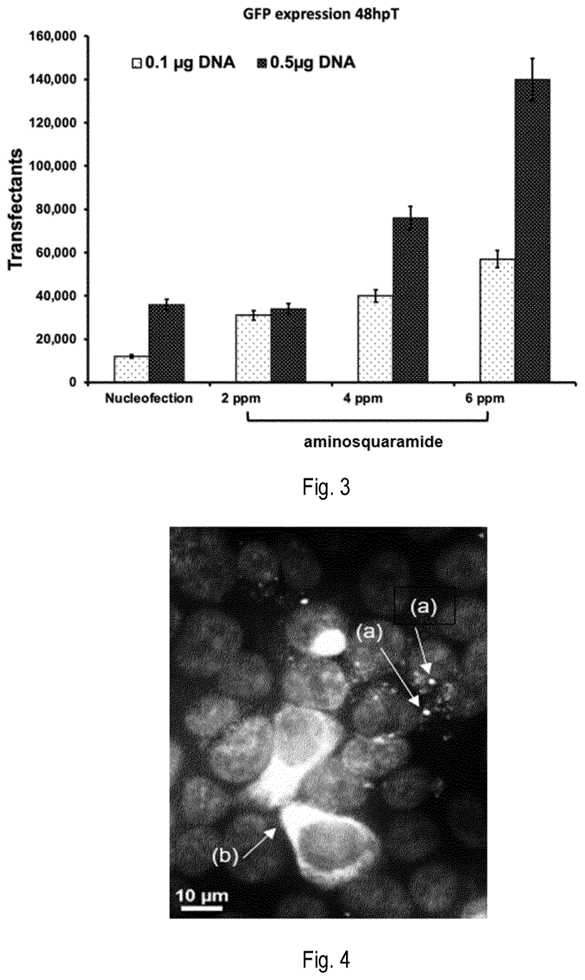

As demonstrated in the results of FIG. 3, superior transfection efficiency of T. cruzi with exogenous DNA using the aminosquaramide compound (mixture 1) of the present invention was achieved, in comparison to the best commercially available procedure.

### Other Protozoan Parasites

The protozoan parasites Trypanosoma brucei, Leishmania, and Plasmodium falciparum were also transfected using the above-standard aminosquaramide protocol disclosed for T. cruzi with exogenous DNA.

### Gene Editing of Extracellular Forms of Protozoan Parasites: Transfection of Nucleic Acids

The gene editing of epimastigote forms of protozoan parasite T. cruzi was performed. The aminosquaramide of the invention (the high molecular weight fraction of mixture 1 obtained by Method B) can be used in combination with the CRISPR/cas9 system to facilitate genome editing using nucleic acids molecules of different length.

A T. cruzi strain that expressed a dual luciferase: mNeonGreen fluorescent protein, in addition to both T7 polymerase and Cas9 proteins was chosen. A gene knock-in strategy was used to switch fluorescence from green to red. With the appropriate gDNA templates and homology donors, it was feasible to integrate the mScarlet gene, replacing mNeonGreen gene. Parasites were transformed with the sgDNA and mScarlet donor PCR products. 70 ng of DNA template and 5 ng of the two gDNAs could complex with 6 ppm of the aminosquaramide of the invention (mixture 1). These amounts are far below those necessary to achieve successful editing with standard commercially available procedures such as electroporation (5 μg of DNA template and 3.5 μg of the two gDNAs).

### Gene Editing of Extracellular Forms of Protozoan Parasites: Transfection of Nucleic Acids and Proteins

The gene editing of epimastigote forms of protozoan parasite T. cruzi was performed. The aminosquaramide of the invention (the high molecular weight fraction of mixture 1 obtained by Method B) can be used in combination with the CRISPR/Cas9 system to facilitate genome editing using nucleic acids molecules of different length and proteins.

Using a T. cruzi strain that expressed constitutively the T7 polymerase, we tagged an endogenous gene with a fluorescent protein (mScarlet). With the appropriate gDNA template for production of the gRNA which will specify the cleavage site to an exogenously delivered Cas9-GFP, and the appropriate DNA template containing the sequence to be used as repair fragment, including the mScarlet tag in frame, we can produce parasites that will incorporate a mScarlet tag into the endogenous gene PF16. Phenotypically, the parasites displayed a red flagellum that can be visualized using epifluorescence microscopy. To achieve this editing, 1 μg of Cas9-GFP (Sigma), 70 ng of DNA template and 5 ng of gDNA, both obtained as PCR products, were allowed to complex with 10 ppm of the aminosquaramide of the invention (the high molecular weight fraction of mixture 1 obtained by Method B) for 30 min in RPMI at room temperature. Then, the mixture was incubated with the parasites in accordance with the transfection protocol shown above. At 72 h post transfection, it was observed that some parasites started to have a red flagellum. The Cas9-GFP protein remained in the parasites for up to 7 days, as it could be visualized by the green fluorescence emission.

### 1.1.5.3. Transfection of Intracellular Pathogens

Transfection of Intracellular Forms of Protozoan Parasite Trypanosoma cruzi

T. cruzi is an obligate intracellular parasite that can infect most mammalian cell types. For this demonstration, MA104 (fetal African green monkey kidney epithelial cells-ATCC CRL-2378.1) was used. These were cultivated to 95-100% confluency in Minimum Essential Medium Eagle (MEM, Sigma), supplemented with 5% Foetal Bovine Serum (FBS), 100 U/ml of penicillin, and 100 μg/ml streptomycin at 37° C. and 5% CO₂.

Tissue culture trypomastigotes (TCTs) of a T. cruzi strain that expressed a mScarlet protein were obtained from previously infected MA104 cells. MA104 cells were infected for 18 h with a multiplicity of infection of 1:10 (cell:parasite). External parasites were then removed by washing in Hank's Balanced Salt Solution (Lonza), and the cells incubated with fresh MEM (Minimum Essential Medium, Sigma-Aldrich), supplemented with 5% FBS) for a further 2-3 days.

On the day of transfection, cells were washed with PBS. The packaging reaction was prepared using RPMI, 500 ng of pTREX-nGFP plasmid and 18 ppm of the aminosquaramide of the invention (mixture 1) and allowed to complex for 30 min at room temperature. Then, the reaction was added to the cells and incubated for 1 h. Afterwards, 2 volumes of complete medium with 155% FBS were added to reach a final concentration of 5%. Transfection was followed up by observation under fluorescent microscopy, where transfected individual amastigotes were expressing simultaneously red and green fluorescent proteins. Selection of transfected parasites was possible by using a host cell line resistant to the drug selectable marker contained in the pTREX-nGFP plasmid (neo—neomycin phosphotransferase).

Transformation of Obligate Intracellular Bacterium Rickettsia canadensis.

Rickettsia canadensis is an obligate intracellular Gram-negative bacterium. They have a short life span (˜20 min) when extracted from host cells (in this case African green monkey kidney—VERO cells). VERO cells were cultivated to 95-100% confluency in RPMI (Sigma), supplemented with 10% Foetal Bovine Serum (FBS), 100 U/ml of penicillin, and 100 μg/ml streptomycin at 37° C. and 5% CO₂. The aminosquaramide of the invention (the high molecular weight fraction of mixture 1 obtained by Method B) protocol can be adapted to transform these microorganisms in a passive manner. Host cells are seeded into 6 well plates and allowed to attach for 4 h, then exposed to a package reaction (plasmid+the high molecular weight fraction of mixture 1 obtained by Method B of the present invention) for 1 h before infection. During this time, bacteria isolated from previously infected cells, can be added to the transfected host cells. After 3 h, the medium (with non-internalized bacteria) is removed and replaced with fresh growth medium. The selective antibiotic (rifampicin) is added 24 h later. Every 5 days, the bacteria were isolated and allowed to re-infected fresh cells, with drug pressure maintained during each passage.

Stable transformation efficiency was assessed by isolating DNA after each passage and monitoring the levels of bacteria (using 16S rDNA primers) and plasmid (using RifR primers) by qPCR. Epifluorescence microscopy was used to visualize GFP-expressing bacteria. Bacteria-specific antibodies was used as a counterstain, and the efficiency of transformation determined by dividing the number of GFP expressing bacterial cells by the total number of bacterial cells within a field of view.

In the absence of aminosquaramide of the invention (the high molecular weight fraction of mixture 1 obtained by Method B) in the package reaction, the plasmid is not detected beyond the second passage.

### 1.1.5.4. In Vivo Transfection of DNA

Transfection of Balb/c mice was performed using the aminosquaramide compound (I) of the present invention, particularly the high molecular weight fraction of mixture 1 obtained by method B.

### Transfection Protocol

A transfection mix was prepared diluting 1 μg of pMAX-GFP in 250 μl DMEM® (Gibco™) and incubating it together with 14 ppm of the above-mentioned mixture 1 of the present invention for 30 min at room temperature.

This protocol was used to prepare the same reaction mentioned above to inoculate into female Balb/c mice (18-20 g of weight and 10 weeks old) by different routes (such as intranasal, intraperitoneal, and intramuscular). 50 μl of transfection mixture were administered 3 times per day during 3 consecutive days. Mice were imaged at different timepoints after the second day of transfection reaction administration using the IVIS Spectrum system (Caliper Life Science), where fluorescence images were obtained using excitation filters set at 465 nm and emission filters at 502 nm for neon green (f-stop: 16, exposure: 8 s). The relative fluorescence was measured from the images by drawing regions of interest (ROIs) using Living Image 4.7.3 software.

### Results

Transfection efficiency was assessed after transfection by fluorescence emission (from the GFP expression). For the in vivo transfection, patches of signal were detected already after the fifth dose and remained stable up to 24 h after last dose.

### 1.2. Mixture 2 of Compounds of Formula (I)-1A-2 and (I)-1B-2

### 1.2.1. Composition

The mixture 2 of the present invention comprises aminosquaramide polymers of formula (I)-1A-2 and (I)-1B-2 as disclosed below:

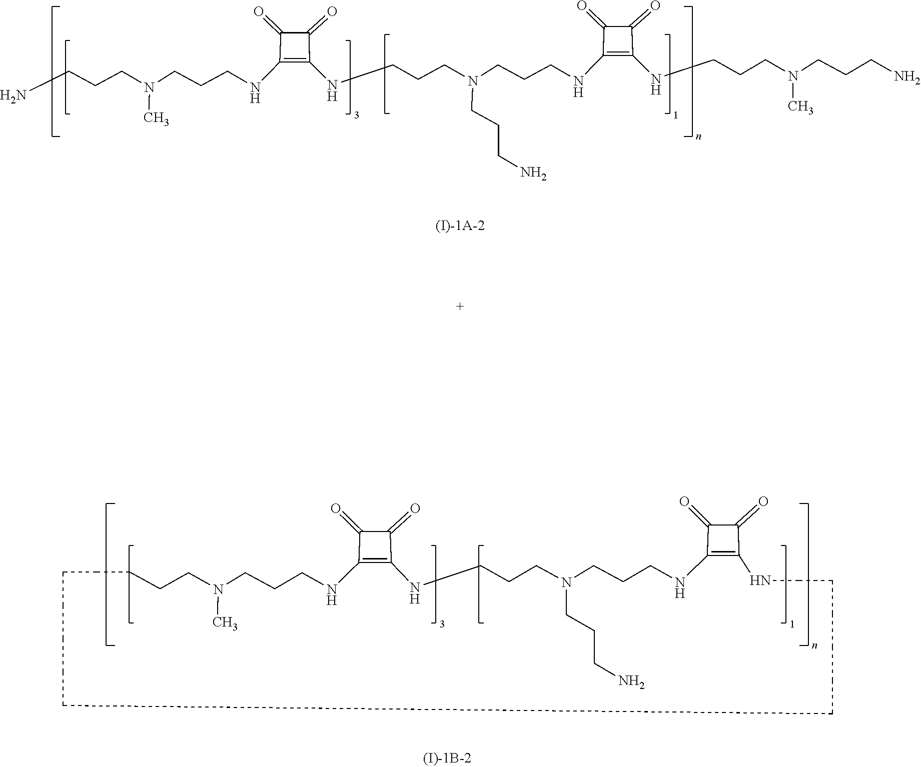

### 1.2.2. Preparation and Purification Process of Mixture 2

Mixture 2 was prepared following the method disclosed below which involves reacting the diamine (V) (obtained as defined above) with the diester (XI):

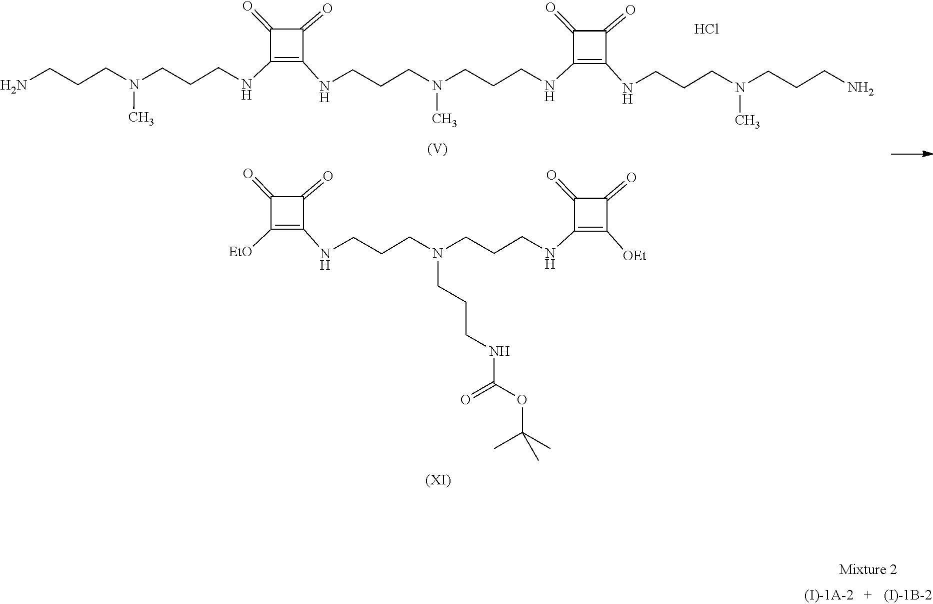

### Synthesis of Diester (XI)

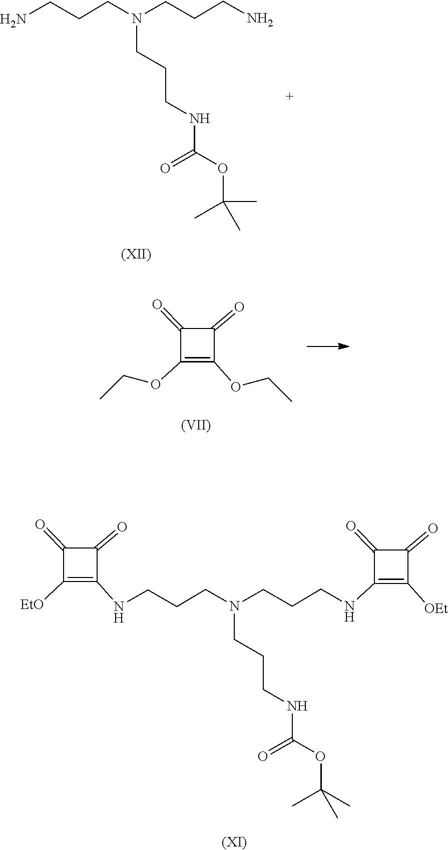

Diester (XI) was synthesized from the corresponding diamine (XII) (diamine XII was prepared following the process disclosed in Bioconjugate Chem., 2014, 25, 1537-1546). A solution of the diamine (XII) (1.04 g, 3.59 mmol) in acetonitrile (45 mL) was added dropwise to a solution of DES (VII) (1.52 g, 8.91 mmol) in 15 mL of acetonitrile, and the mixture was kept under argon atmosphere for an additional 24 h, with stirring at room temperature. The solvent was concentrated under vacuum, affording a brown oil. The resulting oil was purified removing the excess of starting materials by five sequential cycles of dissolution with acetonitrile (5 mL) and precipitation with diisopropyl ether (30 mL) to yield 1.56 g (81%) of diester (XI) as a pale-yellow solid.

### Synthesis and Separation of a Low and a High Molecular Weight Fraction of Mixture 2

Solid Cs₂CO₃ (2.5 g, 7.7 mmol) was added to a suspension of the diamine hydrochloride (V) (1.02 g, 1.31 mmol) in ethanol (12 mL), and the mixture was stirred for 1 h at room temperature. A solution of the diester (XI) (0.87 g, 1.62 mmol) in ethanol (14 mL) was added, and the reaction mixture was kept under argon atmosphere and stirred for an additional 48 h. The resulting solid was filtered and washed with cold ethanol (3×20 mL), cold water (3×20 mL), 30 mL acetone, and dried at room temperature under an active vacuum for 12 h, to afford crude BOC-protected mixture 2 (1.05 g) as a pale-yellow solid. Then, 10 mL 3 M HCl was added to a suspension of the crude BOC-protected mixture 2 that had been obtained (0.97 g) in water (20 mL), and the solution was kept under argon atmosphere for an additional 12 h, with stirring at 50° C. The solvent was concentrated under vacuum, affording the crude mixture 2 as the hydrochloride salt.

Fractionation and purification of a re-dissolved crude mixture 2 as the hydrochloride salt solution obtained above were accomplished through a series of centrifugation filtration steps utilizing an Ortoalresa centrifuge working with Amicon Ultra 15 mL centrifugal filters of 3 kDa MW cut off (Millipore, MA, USA). Briefly, a solution of the above yellow solid (0.53 g) in 30 mL HCl (10⁻³ M) or formic acid (0.1% v/v) was passed through two 3K filters at 3220 g for 40 min (15 mL for each filter). The filtrate solution was discharged and the concentrate was diluted with the acid solution (12 mL) and spun down again. This cycle of dilution-filtration was repeated five times. Then, the isolated concentrate was collected and reduced by rotary evaporation affording the mixture 2 (0.17 g) with a molecular weight distribution >3 kDa.

### 1.2.3. Physico-Chemical Characterization of Mixture 2

The ¹H NMR and ¹³C NMR spectra of the high molecular weight fraction >3 kDa of mixture 2 obtained and purified following the process disclosed in previous section were recorded.

¹H RMN (600 MHz, H₂O/HCOOH 0.1% v/v, coaxial D₂O/DSS): δ (ppm) 1.91, 2.74, 2.93, 3.06, 3.08, 3.17, 3.49

¹³C RMN (150 MHz, H₂O/HCOOH 0.1% v/v, coaxial D₂O/DSS): 5 (ppm 23.4, 27.0, 38.1, 39.1, 39.2, 39.4, 39.5, 39, 67, 36.8, 39.9, 41.6, 42.8, 51.9, 54.9

### 1.3. Mixture 3 of Compounds of Formula (I)-1A-3 and (I)-1B-3

### 1.3.1. Composition

The mixture 3 of the present invention comprises aminosquaramide polymers of formula (I)-1A-3 and (I)-1B-3 as disclosed below:

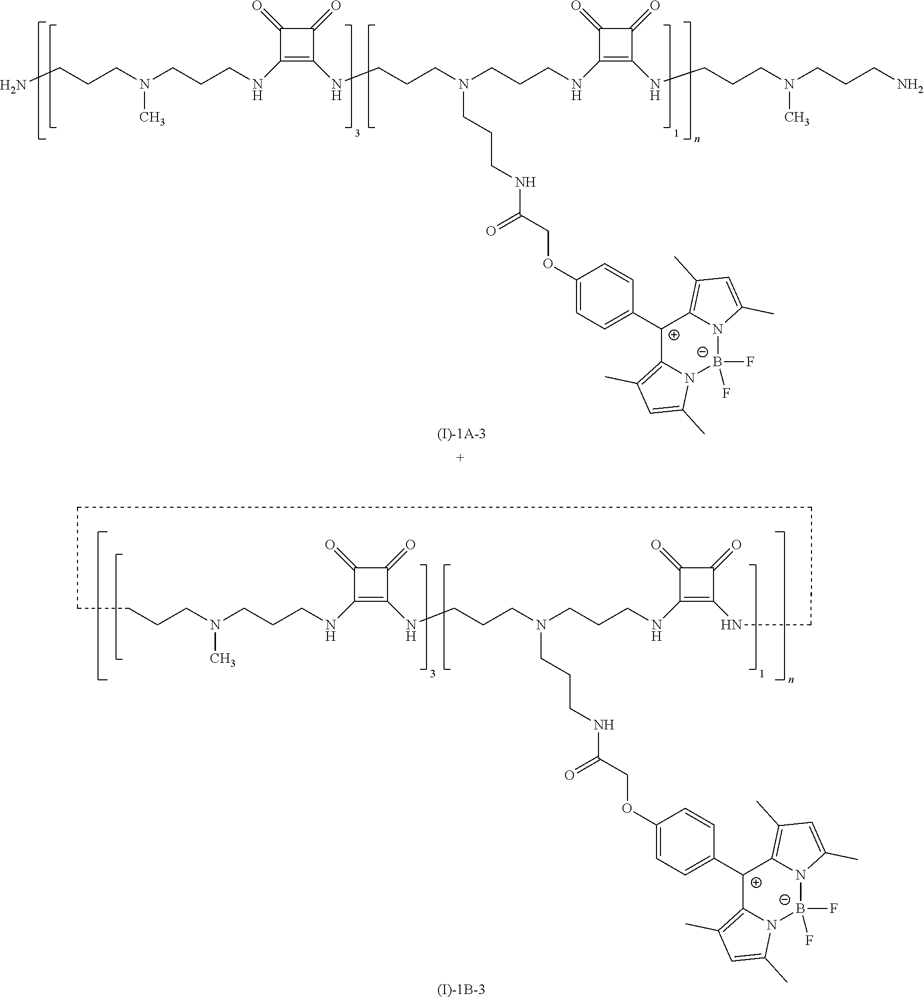

### 1.3.2. Preparation and Purification Process of Mixture 3

Mixture 3 was prepared following the method disclosed below which involved reacting mixture 2 comprising the compounds of formula (I)-1A-2 and (I)-1B-2 as defined above with the compound of formula (XIII):

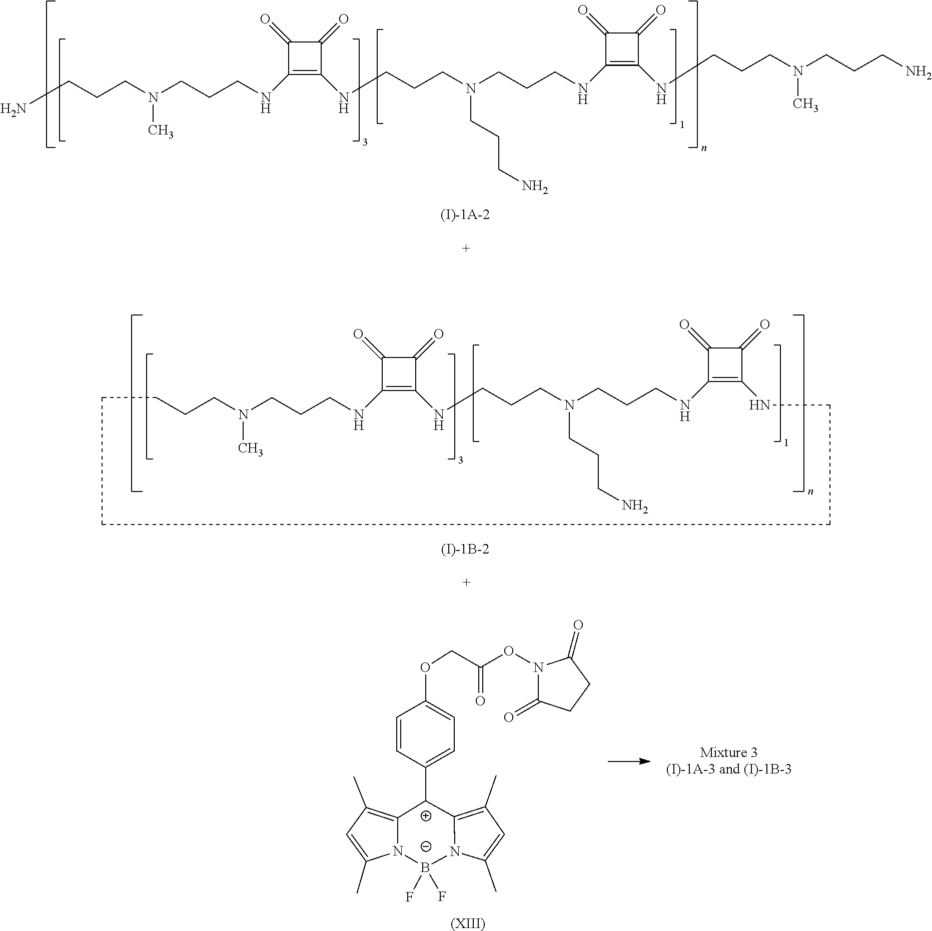

### Synthesis of Compound of Formula (XIII)

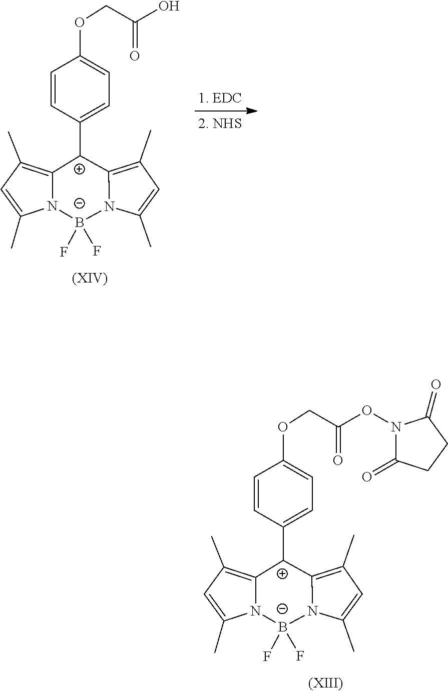

Solid 1-ethyl-3-(3-dimethylaminopropyl)carbodiimide (EDC) (3.56 mg, 0.017 mmol) and N-hydroxy succinimide (NHS) (3.45 mg, 0.030 mmol) were added to the compound of formula (XIV) (6.14 mg, 0.015 mmol; obtained following the process disclosed in Angel Sampedro et al. Bioconjugate Chem., 2014, 25, 1537-1546) dissolved in 600 μL of THE and aqueous buffered solution 3-(N-Morpholino)propane sulfonic acid (MOPS) (0.1 M, pH 6.55) with a ratio of 1:3. The components were mixed and allowed to react for 15 min at room temperature to obtain the compound of formula (XIII) which was directly used in the preparation of mixture 3.

### Synthesis of Mixture 3

Mixture 2 (15 mg) as obtained above dissolved in 1.5 mL of aqueous buffered solution (MOPS 0.1 M, pH 6.88) was added to the mixture obtained in the previous section comprising the compound of formula (XIII) and the reaction was allowed to proceed for 12 h at room temperature in the dark.

The precipitate was removed upon centrifugation and the remain solution was neutralized with NaHCO₃ until the formation of a precipitate. This precipitate was collected by centrifugation, dissolved in H₂O/HCOOH (0.1% v/v), and lyophilized affording mixture 3 as an orange gummy solid (5.97 mg).

### 1.3.3. Physico-Chemical Characterization of Mixture 3

The ¹H NMR and ¹³C NMR spectra of the high molecular weight fraction of mixture 3 obtained and purified following the process disclosed in previous section were recorded. ¹H RMN (600 MHz, H₂O/HCOOH 0.1% v/v, coaxial D₂O/DSS): δ (ppm) 2.6, 2.78, 2.89, 2.91, 3.00, 3.09, 3.20, 3.23, 3.32, 3.33, 3.35, 3.56, 3.58, 3.66, 3.79, 3.81, 3.83, 4.11, 4.14.

The ¹H NMR and ¹³C NMR spectra of mixture 3 were compared with those recorded for starting BOC-protected mixture 2 of the present invention which did not contain the detection moiety. The ¹H and ¹³C NMR spectra of mixture 3 displayed the diagnostic triplet at 3.09 ppm, assigned to the terminal protonated amino methylene groups, and a peak at 39.39 ppm in the ¹³C spectra.

### 1.3.4. Transfection Study

HeK293T cells were transfected with 1 μg pDsRed-Express-C1 (PT3726-5; Cat. No. 632430—(codifying DsRed protein) and 28 ppm of mixture 3 of the present invention obtained above. Mixture 3 and the plasmid were added to 250 μl of RPMI, and after 30 min, the resulting 250 μl transfection mix was added to a well within a 12-well plate, and then 100,000 HEK-293T cells diluted in antibiotic-free medium containing 10% FBS were added dropwise up to a final volume of 1 ml. 24 h later, the medium was replaced with fresh complete medium, and 48 h after the treatment, confocal microscopy images (Inverted Zeiss LSM880 confocal microscope) were taken (cf. FIG. 4).

The images confirmed that the aminosquaramide polymers of formula (I)-1A-3 and (I)-1B-3 according to the present invention, forming part of mixture 3, are capable of transfecting established cell lines and at the same time being detected by fluorescence, due to the presence of detection moieties in the polymeric structure.

### 1.4. Mixture 4 Comprising a Compound of Formula (I)-1A-4 and (I)-1B-4

### 1.4.1. Composition

The mixture 4 comprises a linear aminosquaramide polymer of formula (I)-1A-4 and a cyclic aminosquaramide polymer of formula (I)-1B-4. Specifically, the aminosquaramide polymer of formula (I)-1A-4 of the present invention is a linear polymer where m is 0, X is (CH₂)₃, and where Z is CH₃ in 90% of the repeating units (expressed as monomer A), and (CH₂)₃—NH—CO—O—C(CH₃)₃ in 10% of the repeating units (expressed as monomer B). The aminosquaramide polymer of formula (I)-1B-4 of the present invention comprises a cyclic polymer where m is 0, X is (CH₂)₃, and where Z is CH₃ in the 90% of the repeating units (expressed as monomer A)) and (CH₂)₃—NH—CO—O—C(CH₃)₃ in 10% of the repeating units (expressed as monomer B). Therefore, the aminosquaramide polymer of formula (I)-1A-4 and (I)-1B-4 of the present invention comprises 90% of monomer A and 10% of monomer B randomly distributed.

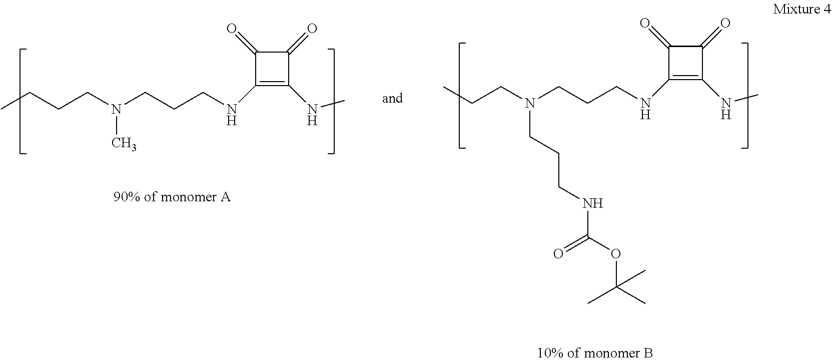

### 1.4.2. Preparation and Purification Process of Mixture 4

3,3′-diamino-N-methyldipropylamine (VI) (0.44 g, 3.1 mmol, 0.9 equivalent) and diamine XII (0.098 g, 0.3 mmol, 0.1 equivalent) in 7.5 mL ethanol were added with stirring to a solution of diethyl squarate (VII) (0.58 g, 3.4 mmol) in 7.5 mL ethanol, at room temperature. A yellowish precipitate formed in seconds and stirring was continued for 4 h at room temperature. The precipitate was collected by filtration, washed with ethanol, and dried. The crude solid was purified by centrifugal filtration following the procedure described in method B above.

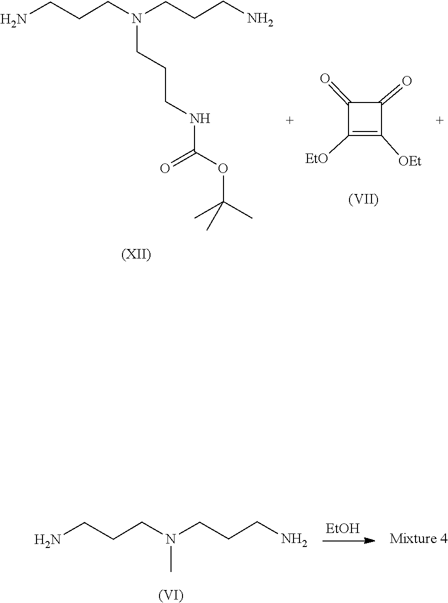

### 1.4.3. Physico-Chemical Characterization of Mixture 4

The ¹H NMR and ¹³C NMR spectra of the mixture 4 obtained and purified following the process disclosed in previous section were recorded.

¹H RMN (600 MHz, H₂O/HCOOH 0.1% v/v, coaxial D₂O/DSS): δ (ppm) 1.39, 1.90, 2.08, 2.90, 3.16, 3.27, 3.66, 8.37

¹³C RMN (150 MHz, H₂O/HCOOH 0.1% v/v, coaxial D₂O/DSS): δ (ppm) 26.8, 27.0, 29.4, 41.7, 42.9, 51.9, 55.0, 64.3, 169.9, 170.8, 183.5—

The ¹H and ¹³C NMR spectra of mixture 4 showed the diagnostic triplet at 3.09 ppm, assigned to the terminal protonated amino methylene groups, and a peak at 39.39 ppm in the ¹³C spectra.

### 2. Aminosquaramide Polymers of Formula (I)-2

### 2.1. Mixture 5 of Compounds of Formula (I)-2A-1 and (I)-2B-1

The mixture 5 of the present invention comprises aminosquaramide polymers of formula (I)-2A-1 and (I)-2B-1 where m is 1; X is —(CH₂)₃—; Z is CH₃; Y is —(CH₂)₂; R₃, R₄, R₅ and R₆ are H, p is 2, and n is from 7 to 25 for (I)-2A-1, and from 7 to 25 for (I)-2B-1.

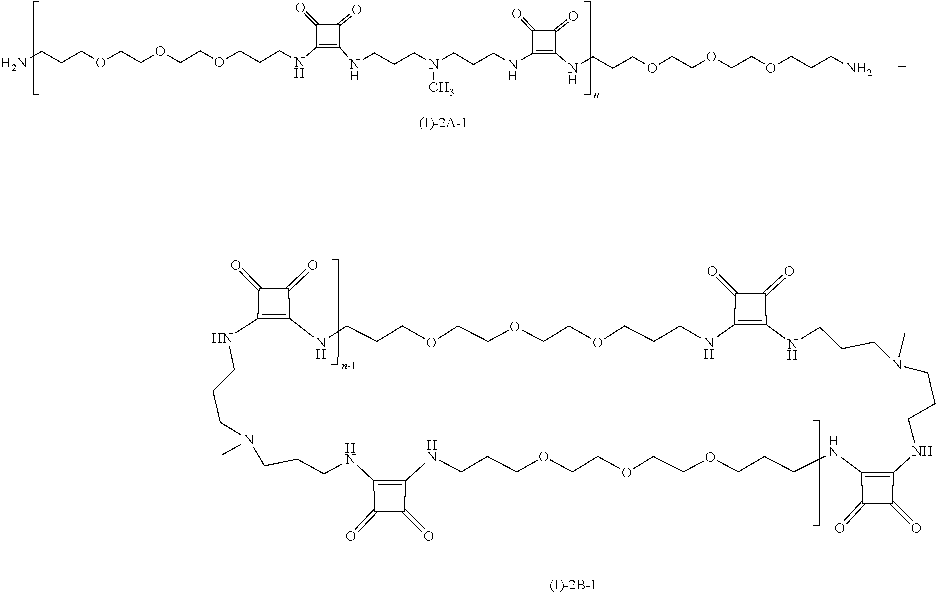

### 2.2. Preparation and Purification Process of Mixture 5

Mixture 5 was prepared and two different fractions (a low molecular weight fraction and a high molecular weight fraction) of 5 were separated.

Mixture 5 was prepared by reacting the diester (IV) (535 mg, 1.4 mmol) and the diamine (XI) (300 mg, 1.4 mmol) in dimethylformamide (DMF) (1.5 mL) and the mixture thus obtained was stirred for 12 hat 115° C. to obtain a precipitate, which was filtered and washed with ethanol.

The precipitate thus obtained was purified by repeating cycles of re-dissolution/precipitation-filtration as disclosed in method B above, using filters Amicon® of 3 and 10 kDa to achieve the low molecular weight fraction of mixture 5 having a numeral average molecular weight of 4100 Da and the high molecular weight fraction having a numeral average molecular weight of 13000 Da. The values of number average weight (Mn) of low- and high-molecular weight fractions of the aminosquaramide compound of mixture 5 the present invention, were determined by GPC-UV. A scheme of the synthesis of mixture 5 is disclosed below:

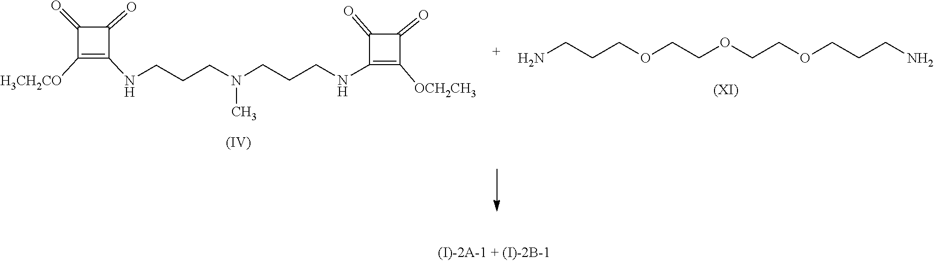

### 2.3. Physico-Chemical Characterization of Mixture 5

The ¹H NMR and ¹³C NMR spectra of the low and high molecular weight fractions of mixture 5 obtained and purified following the process disclosed in the previous section were recorded.

### Low Molecular Weight Fraction of Mixture 5

¹H RMN (600 MHz, H₂O/HCOOH 0.1% v/v, coaxial D₂O/DSS): δ (ppm) 7.72 (b, NH), 3.65 (b, CH₂), 3.28 (b, CH₂), 3.11 (t, CH₂), 2.91 (s, CH₃), 2.09 (b, CH₂), and 1.88 (t, CH₂).

¹³C RMN (150 MHz, H₂O/HCOOH 0.1% v/v, coaxial D₂O/DSS): δ (ppm) 184.5, 171.2, 72.5, 70.8, 56.4, 44.6, 44.2, 42.7, 32.0, and 28.3.

### High Molecular Weight Fraction of Mixture 5

¹H RMN (600 MHz, H₂O/HCOOH 0.1% v/v, coaxial D₂O/DSS): δ (ppm) 7.72 (b, NH), 3.65 (b, CH₂), 3.28 (b, CH₂), 3.11 (t, CH₂), 2.90 (s, CH₃), 2.09 (b, CH₂), 1.88 (t, CH₂).

¹³C RMN (150 MHz, H₂O/HCOOH 0.1% v/v, coaxial D₂O/DSS): δ (ppm) 184.2, 170.6, 72.1, 70.3, 56.0, 44.2, 43.8, 42.3, 32.6, 27.9.

### 2.4. Transfection Study

As mentioned above for aminosquaramides of formula (I)-1 of the present invention, the aminosquaramides of formula (I)-2 of the present invention are also suited to delivery of biologically active molecules into a wide variety of cells, including, but not limited to, cell lines commonly used in research laboratory settings, cultivable protozoan parasites, free-living protists, bacteria, and in addition, cells lines and lineages that are typically considered difficult to transfect. The biologically active transfected molecules retain activity after delivery. The biologically active molecules that can be delivered into cells may be any biological macromolecule molecule of interest, including but not limited to, negatively charged macromolecules. Examples of molecules of interest includes peptides, polypeptides, and nucleic acids (i.e. DNA, RNA, PNA and other synthetic nucleic acids).

### 2.4.1. Transfection of Mammalian Cells in Culture

Preliminary transfection studies have been carried out with the aminosquaramides of formula (I)-2 of the present invention and its low and high molecular weight fractions separately in HEK293 cells. The same transfection protocol used for HEK293 cells with the aminosquaramide compound (I)-1 of the present invention as defined above, were used with the high molecular weight fraction of mixture 5 of the present invention. As positive control, the same transfection test was also performed with the standard commercial reagent Lipofectamine2000® (Invitrogen) including the same corresponding macromolecules to be transfected, following the manufacturer's instructions.

The results of the transfection test in HEK293 cells showed a positive transfection outcome using the high molecular weight fraction of mixture 5 at 4 and 10 ppm. The plate reader allowed quantification of the GFP expression results.

The GFP expression obtained with the use of the high molecular weight fraction of mixture 5 of the present invention (10 ppm) was 74% in comparison with that obtained with the Lipofectamine2000® (standard positive control); and 64% of viable cells in comparison with the one obtained with negative control (not transfecting agent). Treatment with 4 ppm of high molecular weight fraction of mixture 5 afforded 70% of expression in comparison with that obtained with Lipofectamine 2000® (standard positive control) and 90% of viable cells in comparison with the one obtained with negative control (not transfecting agent). Furthermore, all transfected cells obtained using the aminosquaramide polymer of the present invention were viable. Meanwhile, most of the transfected cells obtained using Lipofectamine were not viable. Therefore, these results show that the aminosquaramide polymers of the present invention are appropriate for being used as transfecting agent, without compromising the viability of the transfected cell and the inherent activity of the molecule of interest contained in it.

### 3. Aminosquaramide Polymers Comprising Monomers of Formula a and Monomers of Formula B Randomly Distributed

### Example 1: Aminosquaramide Polymer Comprising 75% Monomer of Formula a and 25% Monomer of Formula B Random

Monomer A: X is —(CH₂)₃—; Z is —CH₃
Monomer B: X is —(CH₂)₃—; Y is —CH₂—; and p is 2

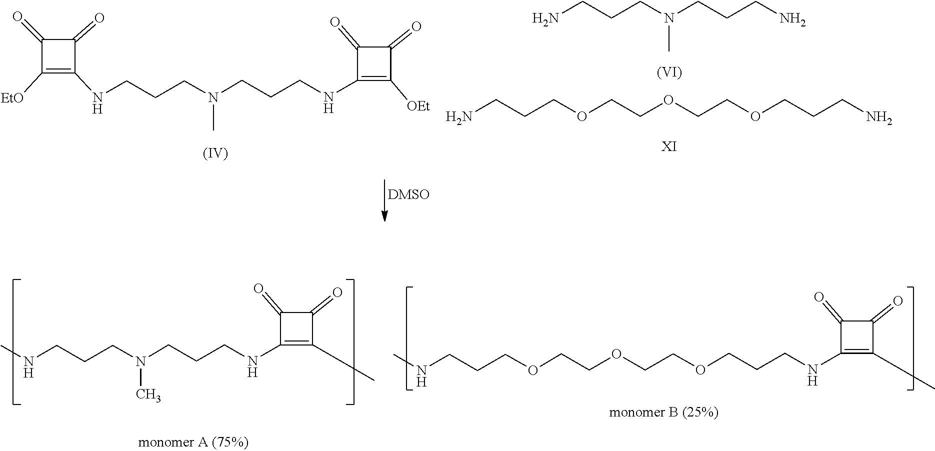

A mixed solution of amines (VI) (98.8 mg, 0.68 mmol) and (XI) (150 mg, 0.68 mmol) in 1.0 mL of DMSO was added to a stirred solution of diester (IV) (535 mg, 1.36 mmol) in 1.5 mL of DMSO at 100° C. Half an hour after the addition, the product precipitate stops the stirring of the reaction. The resulting solid was filtered and washed with 50 mL of ethanol, 30 mL of acetone and dried under vacuum. The high molecular weight (HMW) fraction was obtained after 5 cycles of filtration through 10 kDa cut off AMICON filters using a solution of formic acid 0.1% v/v in water.

¹H-NMR spectrum (D₂O/0.1% HCOOH 600 MHz) δ (ppm): 8.39, 4.89, 3.76, 3.70, 3.42, 3.33, 3.01, 2.18, 1.98. ¹³C-NMR spectrum (D₂O/0.1% HCOOH 150 MHz) δ (ppm): 183.1, 171.5, 171.4, 169.6, 71.2, 70.9, 69.4, 55.0, 54.9, 54.8, 43.1, 42.7, 41.5, 41.4, 41.3, 31.6, 26.9.

### Example 2: Aminosquaramide Polymer Comprising 50% Monomer of Formula a and 50% Monomer of Formula B Random

Monomer A: X is —(CH₂)₃—; Z is —CH₃
Monomer B: X is —(CH₂)₃—; Y is —CH₂—; and p is 2

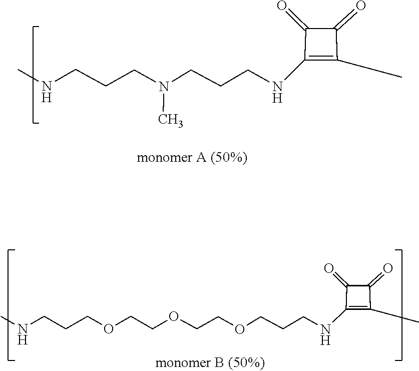

A solution of the amine (XI) (300 mg, 1.37 mmol) in 1.0 mL of DMSO was added to a stirred solution of diester (IV) (535 mg, 1.36 mmol) in 1.5 mL of DMSO at 90° C. After 12 hours the mixture was poured into 25 mL of ethanol to force the precipitation of the polymer. The resulting solid was filtered and washed with 50 mL of ethanol, 30 mL of acetone and dried under vacuum. The high molecular weight (HMW) fraction was obtained after 5 cycles of filtration through 10 kDa cut off AMICON filters using a solution of formic acid 0.1% v/v in water.

¹H-NMR spectrum (D₂O/0.1% HCOOH 600 MHz) δ (ppm): 8.40, 4.89, 3.75, 3.70, 3.48, 3.34, 3.02, 2.20, 1.98. ¹³C-NMR spectrum (D₂O/0.1% HCOOH 150 MHz) δ (ppm): 182.8, 169.7, 169.0, 71.1, 70.9, 69.4, 55.0, 43.1, 42.7, 41.3, 31.6, 26.9.

### Example 3. Aminosquaramide Polymers Comprising Monomer of Formula a and Monomer of Formula B Randomly Distributed

Monomer A9: X is —(CH₂)₃—; Z is —CH₃
Monomer A10: X is —(CH₂)₃—; Z is —(C₂-C₁₂)alkylene-O—(Y—O)—(C₁-C₁₂)alkyl; —(C₂-C₁₂)alkylene is —(CR₃R₄)—; Y is —(CH₂)₂—; R₃ and R₄ are —CH₃ and p is 1.

The preparation process of the aminosquaramide polymer is disclosed herein below:

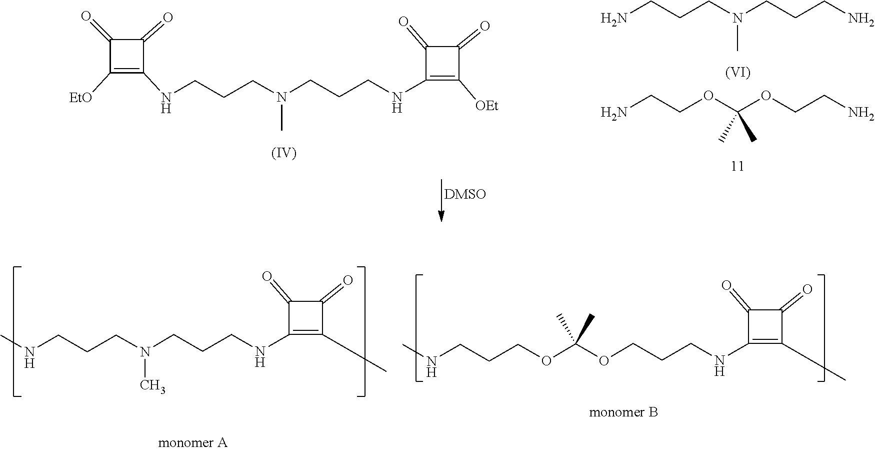

A mixed solution of amines (VI) (158 mg, 1.09 mmol) and (11) (44 mg, 0.27 mmol) in 1.0 mL of DMSO was added to a stirred solution of diester (IV) (535 mg, 1.36 mmol) in 1.5 mL of DMSO at 100° C. Half an hour after the addition, the precipitation of the product makes the stirring impossible. The resulting solid was filtered and washed with 50 mL of ethanol, 30 mL of acetone and dried under vacuum (0.576 g) to obtain a aminosquaramide polymer of Example 3-A comprising 90% of monomer A and 10% of monomer B.

¹H-NMR spectrum (DMSO-d, 600 MHz) δ (ppm): 7.57, 3.71, 3.55, 2.74, 2.70, 2.50, 2.30, 1.84, 1.54, 1.47.

The same process as mentioned above for the preparation of the aminosquaramide of Example 3-A was followed for the preparation of the aminosquaramide polymer Example 3-B having 95% of the monomer A and 55 of the monomer B, but using the amount of compounds VI, VI, and (11) specified in the Table below:

| Aminosquaramide | | | | | |
|---|---|---|---|---|---|
| | Monomer | Monomer | | | |
| | A | B | | | |
| Ex- | % by | % by | Starting material | | |
| ample | weight | weight | VI | 11 | IV |
| Ex. | 95 | 5 | 177.8 mg | 22 mg | 535 mg |
| 3-B | | | (1.22 mmol) | (0.136 mmol) | (1.36 mmol) |

### 4. Aminosquaramide Polymers Comprising 100% of Different Monomer of Formula A

### Example 4. Aminosquaramide Polymers Comprising 25% of Monomer A1 and 75% of Monomer of Formula A2 Block Copolymer Distributed

Monomer A1: X is —(CH₂)₃—; Z is —CH₃
Monomer A2: X is —(CH₂)₃—; Z is —(C₂-C₁₂)alkenyl-O—(Y—O)—(C₂-C₁₂)alkenyl-NR₁₁R₁₃; —(C₂-C₁₂)alkenyl is —(CH₂)₃—; Y is —(CH₂)₂—; R₁₁ is H and R₁₃ is H.

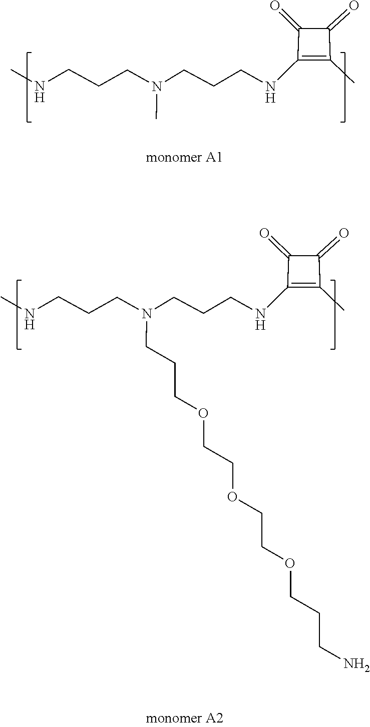

The preparation process is disclosed herein below:

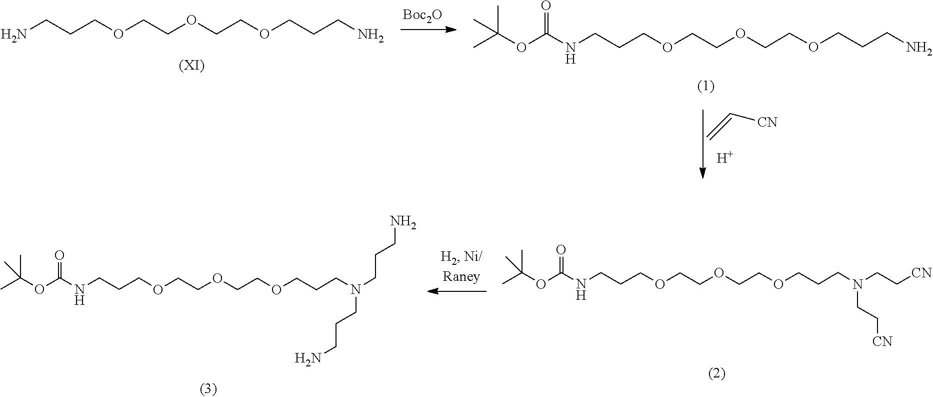

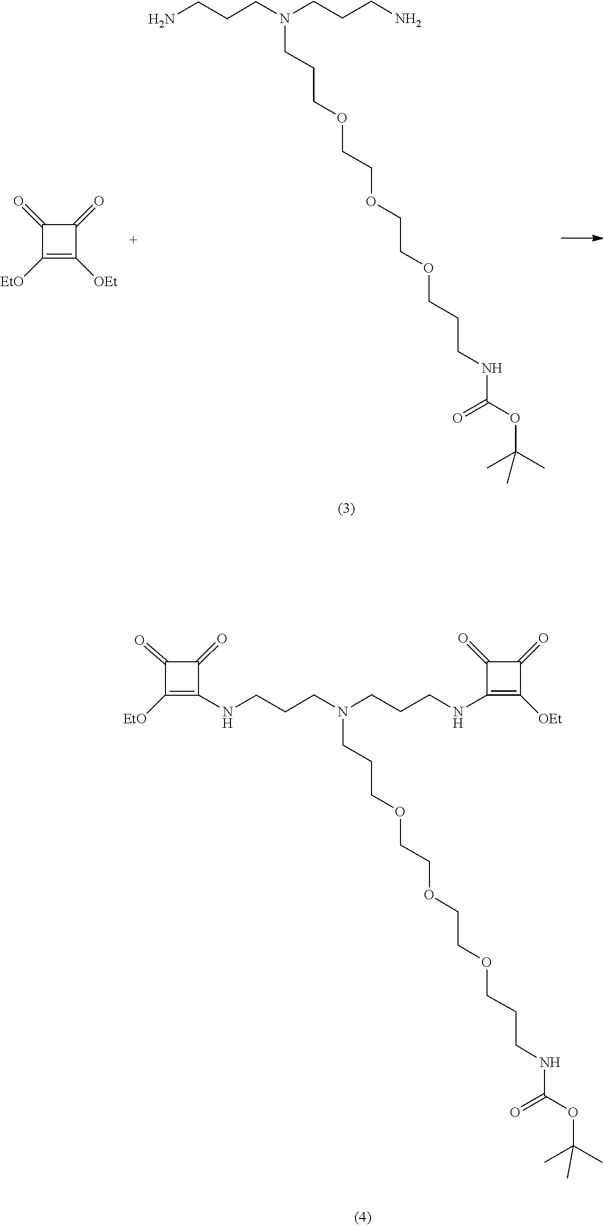 [SMILES: CCOc1c(OCC)c(=O)c1=O ; CC(C)(C)OC(=O)NCCCOCCOCCOCCCN(CCCN)CCCN ; CCOc1c(NCCCN(CCCNc2c(OCC)c(=O)c2=O)CCCOCCOCCOCCCNC(=O)OC(C)(C)C)c(=O)c1=O]

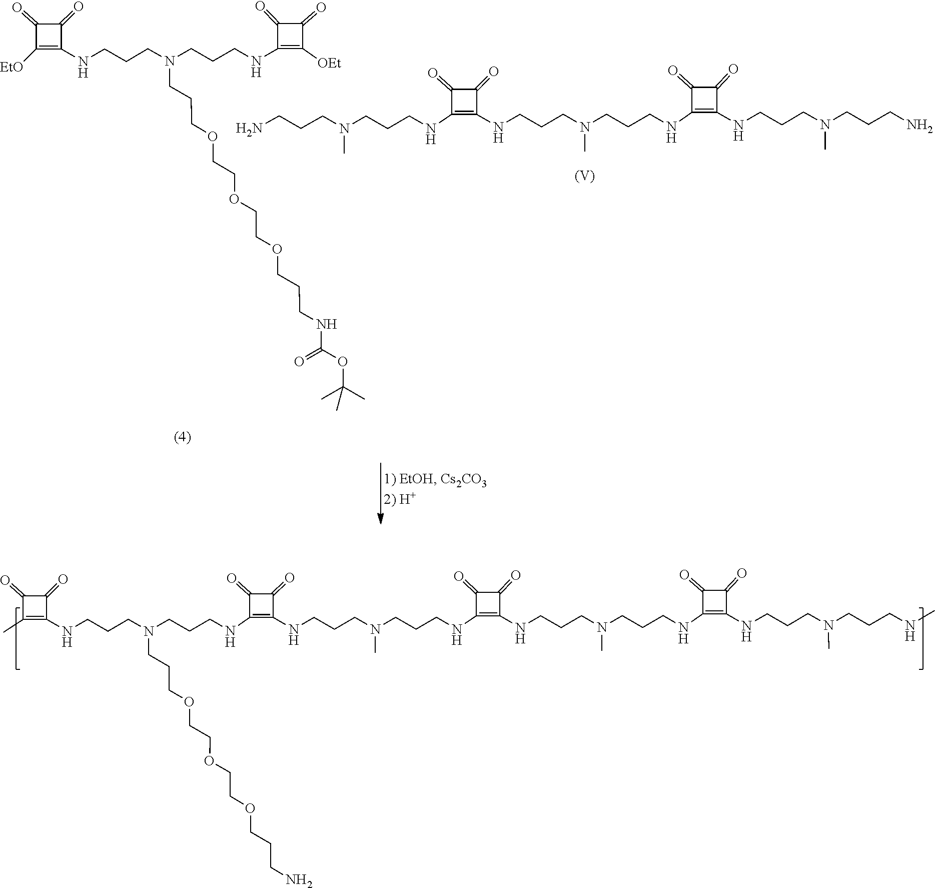

### Synthesis of the Compound (2)

The amine (1) was synthesized according to the process disclosed in Macromolecules, 2018, 51, 12, 4688-4693. A solution of the amine (1) (3.0 g, 9.4 mmol) in acrylonitrile (25 mL, 20.25 g, 382 mmol) and acetic acid (0.54 mL, 0.56 g, 9.36 mmol) was heated at reflux for 24 hours. Then, the solvent was removed under vacuum and the raw material was dissolved in chloroform (25 mL) and poured into concentrated ammonia (5 mL). The organic layer was extracted and washed three times with water, and it was finally dried over anhydrous Na₂SO₄. After filtration, solvent was evaporated to give the (2) nitrile as a yellow oil (3.9 g, 98%).

¹H-NMR spectrum (CDCl₃, 300 MHz) δ (ppm): 7.26, 3.63, 3.21, 2.84, 2.64, 2.49, 1.76, 1.69, 1.44.

HRMS: Calculated C₂₁H₃₈N₄O₅Na⁺ [M+Na]⁺=449.2740. Found: [M+Na]⁺=449.2734.

### Synthesis of the Amine Compound (3)

The nitrile (2) (1.2 g, 2.9 mmol) was dissolved in ethanolic NaOH (1.4 M) and introduced into a 500 mL hydrogenation vessel containing Raney-Ni catalyst (2.4 g). The mixture was hydrogenated at 45 psi overnight at room temperature. The catalyst was filtered through Celite®521 and washed with 95% EtOH. After diluting the filtrate with water (5 mL), EtOH was evaporated and the residue extracted with CH₂Cl₂ (3×30 mL). The organic layers were dried with Na₂SO₄ and the solvent evaporated in vacuo yielding the amine (3) (1.3 g) as a yellow oil (74%).

¹H-NMR spectrum (CDCl₃, 300 MHz) δ (ppm): 7.26, 5.07, 3.48, 3.21, 2.69, 2.47, 1.78, 1.71, 1.58.

HRMS: Calculated C₂₁H₄₇N₄O₅⁺ [M+H]⁺=435.3541. Found: [M+H]⁺=435.3543.

### Synthesis of Diester Compound (4)

A solution of the diamine (3) (0.6 g, 1.38 mmol) in acetonitrile (15 mL) was added dropwise to a solution of DES (VII) (0.7 g, 4.14 mmol) in 5 mL of acetonitrile, and the mixture was kept under argon atmosphere for an additional 24 h, with stirring at room temperature. The solvent was concentrated under vacuum, affording a brown oil. The resulting oil was purified by Al₂O₃ column chromatography (100:0 DCM:MeOH to 95:5 DCM:MeOH) yielding 0.41 g (43%) of diester (4) as a brown oil.

¹H-NMR spectrum (CDCl₃, 300 MHz) δ (ppm): 7.26, 4.78, 3.63, 3.60, 3.20, 1.73, 1.65, 1.43.

HRMS: Calculated C₃₃H₅₅N₄O₁₁+[M+H]⁺=683.3862. Found: [M+H]⁺=683.3864.

### Synthesis of Aminosquaramide of Example 4

Solid Cs₂CO₃ (0.955 g) was added to a suspension of the diamine hydrochloride (V), (0.355 g, 0.497 mmol) in ethanol (5 mL) and the mixture was stirred for 1 h at room temperature. A solution of the BOC-diester (4) (0.407 g, 0.596 mmol) in 5 mL ethanol was added, and the reaction mixture was kept under argon atmosphere and stirred for an additional 48 h. The resulting solid was filtered and washed with cold ethanol (3×20 mL), cold water (3×20 mL), 30 mL acetone, and dried at room temperature under an active vacuum for 12 h, to afford crude BOC-protected mixture (0.397 g) as a pale-yellow solid. Then, 0.78 mL 3 M HCl was added to a suspension of the crude BOC-protected mixture that had been obtained (0.397 g) in water (10 mL), and the solution was kept under argon atmosphere for an additional 12 h, with stirring at 50° C. The solution was treated with concentrated ammonia until pH 5 is reached and then it was passed through a 3 kDa filter at 3220 g for 40 min. The filtrate solution was discharged, and the concentrate was diluted with the acid solution (12 mL) and spun down again. This cycle of dilution-filtration was repeated five times. Then, the isolated concentrate was collected, and it was passed through a 10 kDa filter at 3220 g for 40 min thus repeating the same procedure than with 3 kDa filter. Finally, the product was obtained by lyophilization of the concentrated solution affording a polymeric mixture with a molecular weight distribution over 10 kDa.

### Spectrum of the BOC-Protected Raw Material

¹H-NMR (D₂O/0.1% HCOOH 600 MHz) δ (ppm): 8.39, 4.66, 3.64, 3.54, 3.26, 3.10, 2.90, 2.06, 1.72, 1.38

¹³C-NMR (D₂O/0.1% HCOOH 150 MHz) δ (ppm): 183.4, 171.9, 169.9, 71.2, 71.0, 70.1, 69.4, 55.0, 52.0, 42.8, 41.6, 29.4, 27.0.

Spectrum after Deprotection and Filtration

¹H-NMR (D₂O/0.1% HCOOH 600 MHz) δ (ppm): 8.45, 4.89, 3.78, 3.42, 3.34, 3.21, 3.01, 2.18, 2.05.

¹³C-NMR (D₂O/0.1% HCOOH 150 MHz) δ (ppm): 183.2, 170.3, 170.1, 169.7, 71.1, 71.0, 70.9, 69.8, 69.2, 54.8, 51.7, 42.7, 41.5, 39.1, 28.1, 26.9, 25.0.

### Example 5. Aminosquaramide Polymers Comprising 90% of Monomer A3 and 10% of Monomer of Formula A4 Randomly Distributed

Monomer A3: X is —(CH₂)₃—; Z is —CH₃
Monomer A4: X is —(CH₂)₃—; Z is —(C₁-C₁₂)alkyl optionally substituted with —NH₂.

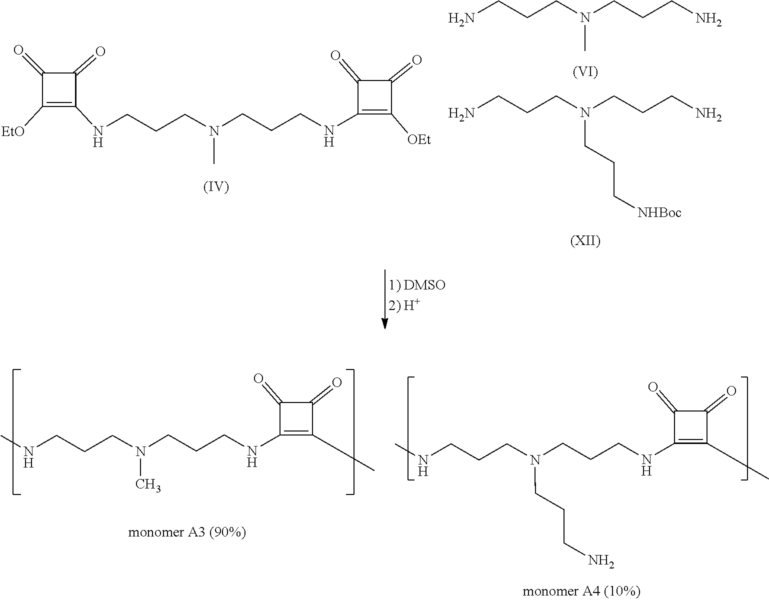

A mixed solution of amines (VI) (178 mg, 1.224 mmol) and (XII) (39 mg, 0.136 mmol) in 1.0 mL of DMSO was added to a stirred solution of diester (VI) (535 mg, 1.36 mmol) in 1.5 mL of DMSO at 100° C. Half an hour after the addition, the precipitation of the product makes the stirring impossible. The resulting solid was filtered and washed with 50 mL of ethanol, 30 mL of acetone and dried under vacuum (0.559 g). Then, 0.60 mL of 3 M HCl was added to a suspension of the crude BOC-protected mixture that had been obtained (0.299 g) in water (10 mL), and the solution was kept under argon atmosphere for an additional 12 h, with stirring at 50° C. The solution was treated with concentrated ammonia until pH 5 is reached and then it was passed through a 10 kDa filter at 3220 g for 40 min. The filtrate solution was discharged, and the concentrate was diluted with the acid solution (12 mL) and spun down again. This cycle of dilution-filtration was repeated five times. Finally, the product was obtained by lyophilization of the concentrated solution affording a polymeric mixture with a molecular weight distribution over 10 kDa.

¹H RMN (600 MHz, H₂O/HCOOH 0.1% v/v, coaxial D₂O/DSS): δ (ppm) 1.91, 2.74, 2.93, 3.06, 3.08, 3.17, 3.49

¹³C RMN (150 MHz, H₂O/HCOOH 0.1% v/v, coaxial D₂O/DSS): 5 (ppm 23.4, 27.0, 38.1, 39.1, 39.2, 39.4, 39.5, 39, 67, 36.8, 39.9, 41.6, 42.8, 51.9, 54.9

### Example 6. Aminosquaramide Polymers Comprising 75% of Monomer A5 and 25% of Monomer of Formula A6 Randomly Distributed

Monomer A5: X is —(CH₂)₃—; Z is —CH₃
Monomer A6: X is —(CH₂)₃—; Z is —(C₁-C₁₂)alkyl

The preparation process of the starting material (7) and the aminosquaramide polymer of Example 5 is disclosed herein below:

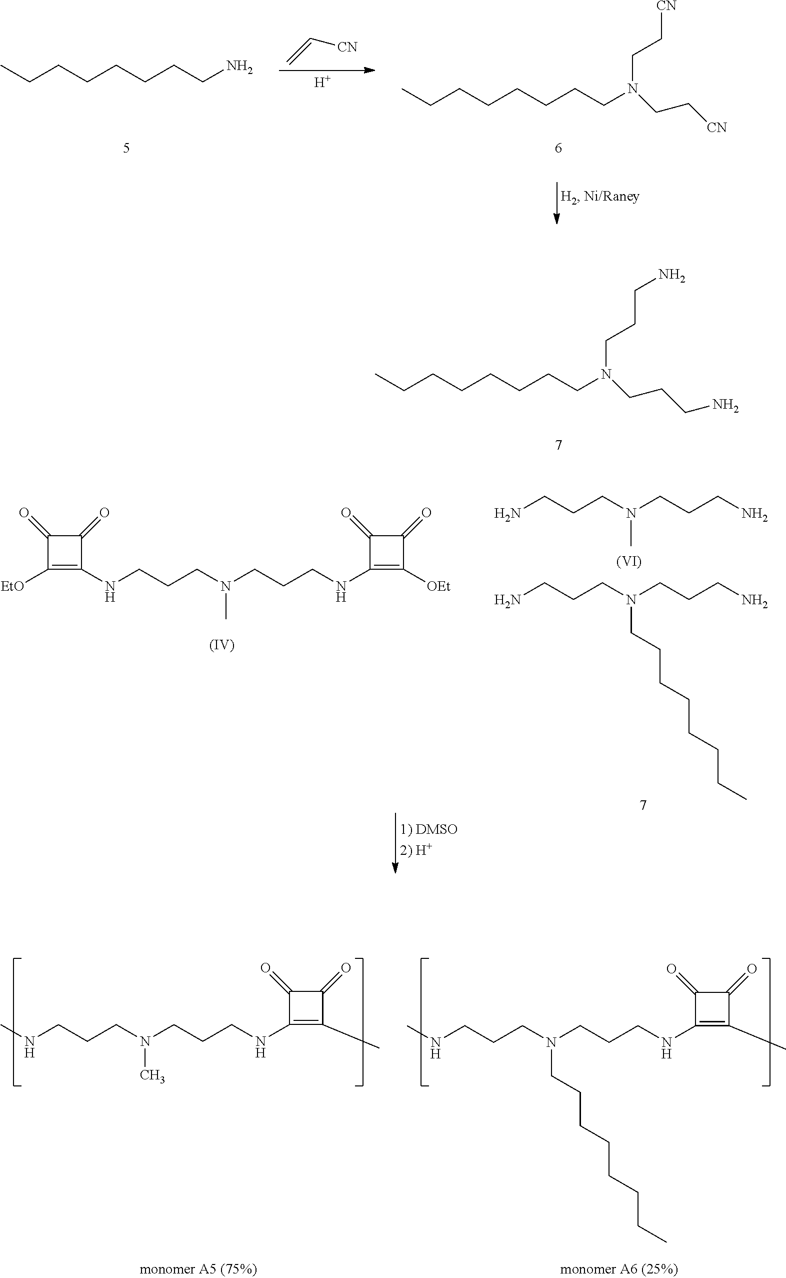

### Synthesis of Nitrile (6)

A solution of the 1-octilamine (1.5 g, 11.6 mmol) in acrylonitrile (25 mL, 20.25 g, 382 mmol) and acetic acid (1 mL, 1.05 g, 17.5 mmol) was heated at reflux for 24 hours. Then, the solvent was removed under vacuum and the raw material was dissolved in chloroform (25 mL) and poured into concentrated ammonia (5 mL). The organic layer was extracted and washed three times with water, and it was finally dried over anhydrous Na₂SO₄. After filtration, solvent was evaporated to give the (6) nitrile as a yellow oil (2.7 g, 98%).

¹H-NMR spectrum (CDCl₃, 300 MHz) δ: 2.861, 2.522, 2.463, 1.447, 1.277, 0.883 HRMS calculated for C₁₄H₂₆N₃⁺ [M+H]⁺=236.2121. Found: [M+H]⁺=236.2115.

### Synthesis of Amine (7)

The nitrile (6 (2.7 g, 11.3 mmol) was dissolved in ethanolic NaOH (1.4 M) and introduced into a 500 mL hydrogenation vessel containing Raney-Ni catalyst (2 g). The mixture was hydrogenated at 45 psi overnight at room temperature. The catalyst was filtered through Celite®521 and washed with 95% EtOH. After diluting the filtrate with water (5 mL), EtOH was evaporated and the residue extracted with CH₂Cl₂ (3×30 mL). The organic layers were dried with Na₂SO₄ and the solvent evaporated in vacuo yielding the amine (7) (2.0 g, 8.4 mmol) as a yellow oil (74%).

¹H-NMR spectrum (CDCl₃, 300 MHz) δ (ppm): 2.749, 2.444, 2.374, 1.581, 1.463, 1.263, 0.878

¹³C-NMR spectrum (CDCl₃, 150 MHz) δ (ppm): 55.8, 53.5, 42.4, 33.4, 32.6, 31.2, 30.9, 29.2, 28.6, 24.3, 15.7 HRMS calculated for C₁₄H₃₄N₃⁺ [M+H]⁺=244.2747. Found: [M+H]⁺=244.2744.

### Synthesis of Aminosquaramide Polymer of Example 6

A mixed solution of amines (VI) (98.8 mg, 0.68 mmol) and (7) (165.5 mg, 0.68 mmol) in 1.0 mL of DMSO was added to a stirred solution of diester (VI) (535 mg, 1.36 mmol) in 1.5 mL of DMSO at 100° C. Half an hour after the addition, the precipitation of the product makes the stirring impossible. The resulting solid was filtered and washed with 50 mL of ethanol, 30 mL of acetone and dried under vacuum (0.565 g). The high molecular weight (HMW) fraction was obtained after 5 cycles of filtration through 10 kDa cut off AMICON filters using a solution of formic acid 0.1% v/v in water.

¹H-NMR (D₂O/0.1% HCOOH 600 MHz) δ (ppm): 8.45, 4.89, 3.75, 3.38, 3.26, 3.00, 2.17, 1.75, 1.41, 1.31, 0.92.

¹³C-NMR (D₂O/0.1% HCOOH 150 MHz) δ (ppm): 183.2, 170.2, 169.4, 54.8, 51.5, 42.7, 41.5, 32.6, 29.7, 27.2, 26.9, 26.6, 24.5, 23.6, 15.0.

### Example 7. Aminosquaramide Polymers Comprising Monomer of Formula A7 and Monomer of Formula A8 Randomly Distributed

Monomer A7: X is —(CH₂)₃—; Z is —CH₃
Monomer A8: X is —(CH₂)₃—; Z is —(C₂-C₁₂)alkylene-O—(Y—O)—(C₁-C₁₂)alkyl; —(C₂-C₁₂)alkylene is —(CH₂)₂—; Y is —(CH₂)₂—; and —(C₁-C₁₂)alkyl is —CH₃

The preparation process of the starting material (10) and the aminosquaramide polymer is disclosed herein below:

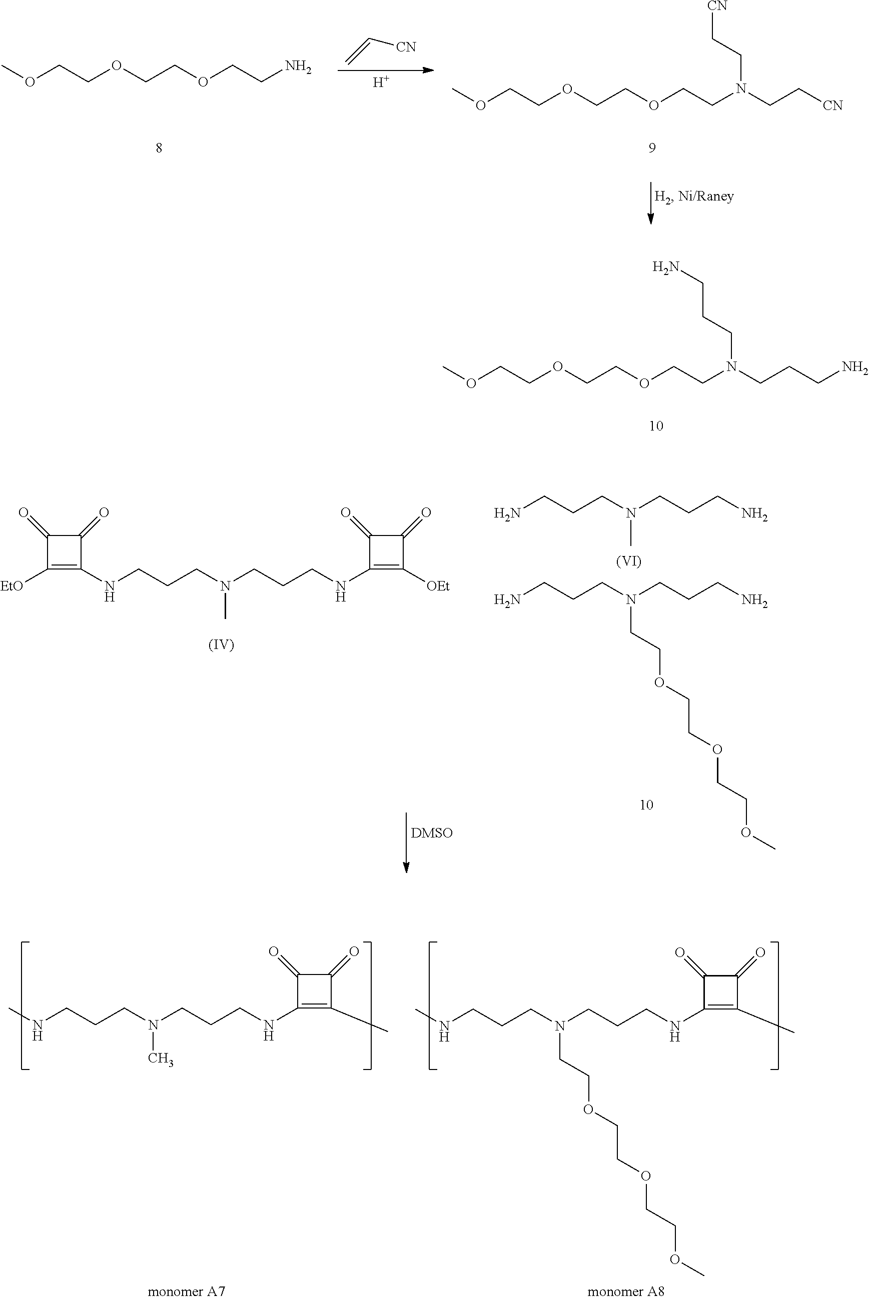

### Synthesis of Nitrile 9

1.90 g (9.2 mmol) of amine 8, 0.63 g (10.53 mmol) of acetic acid were dissolved in a round bottom flask in 18 mL of acrylonitrile and the mixture was heated at 95° C. for 24 h. Then, the solvent was evaporated under reduced pressure. The residue was mixed with 8 ml of dichloromethane, 3 ml of concentrated NH₃ and 5 ml of H₂O. The organic phase was decanted and aqueous phase was extracted with dichloromethane (3×10 ml).

The organic extracts were combined, and dried with NaSO₄ and filtered. The solvent was removed under reduced pressure to afford the dinitrile 9 (1.98 g) as an orange oil. Yield 69%

¹H RMN (300 MHz, CDCl3, ppm) δ; 2.51; 2.81; 2.97; 3.38; 3.65.

¹³C RMN (300 MHz, CDCl3, ppm) δ; 155.5, 118.1, 77.4, 70.1, 70.0, 69.7, 67.4, 49.4, 29.2, 27.9, 27.0, 16.5. (IR, cm-1): 2866.82; 2246.14; 1454.86; 1350.74; 1099.83.

HRMS-ESI(+) calculated for [9+Na]⁺ 336.18938. Found [16+Na]⁺ 336.18907 uma.

### Synthesis of Amine 10

The nitrile 9 (1.77 g, 5.65 mmol) was dissolved in a 15 ml of ethanolic NaOH (1.4 M) and introduced into a 500 mL hydrogenation vessel containing Raney-Ni catalyst (2 g). The mixture was hydrogenated at 45 psi overnight at room temperature. The catalyst was filtered through Celite®521 and washed with 95% EtOH. After diluting the filtrate with water (5 mL), EtOH was evaporated and the residue extracted with CH₂Cl₂ (3×70 mL). The organic layers were dried with Na₂SO₄ and the solvent evaporated in vacuo yielding the amine (10) (0.956 g) as a pale orange oil (53%).

¹H RMN (300 MHz, CDCl3, ppm) δ; 1.60; 2.52; 2.63; 2.73; 3.37; 3.65. ¹³C RMN (300 MHz, CDCl3, ppm) δ; 17.9; 29.7; 39.9; 51.9; 52.7; 56.5; 58.3; 69.1; 69.8; 69.9; 71.2; 76.4; 76.8; 77.3.

¹³C-NMR (CDCl₃, 150 MHz) δ (ppm): 157.7, 78.9, 78.6, 78.4, 72.2, 72.2, 72.1, 71.8, 71.8, 71.7, 71.5, 71.1, 53.7, 52.6, 42.3, 40.0, 31.8, 31.2, 30.1, 28.7

(IR, cm⁻¹): 2863.85; 1458.25; 1099.40 HRMS-ESI(+): Calculated for [10+H]+322.27004 uma. Found [10+H]⁺ 322.26974 uma

### Synthesis of Aminosquaramide Polymer of Example 7

A mixed solution of amines (VI) (165 mg, 1.13 mmol) and (10) (39 mg, 0.14 mmol) in 1.0 mL of DMSO was added to a stirred solution of diester (IV) (497 mg, 1.26 mmol) in 1.5 mL of DMSO at 100° C. Half an hour after the addition, the precipitation of the product makes the stirring impossible. The resulting solid was filtered and washed with 50 mL of ethanol, 30 mL of acetone and dried under vacuum. The high molecular weight (HMW) fraction was obtained after 5 cycles of filtration through 10 kDa cut off AMICON filters using a solution of formic acid 0.1% v/v in water to obtain a aminosquaramide polymer of Example 7-A comprising 90% of monomer A7 and 10% of monomer A8.

¹H-NMR spectrum (D₂O/0.1% HCOOH 600 MHz, 600 MHz) δ (ppm): 8.42, 4.89, 3.76, 3.45, 3.42, 3.33, 3.00, 2.81, 2.18.

¹³C-NMR spectrum (D₂O/0.1% HCOOH 600 MHz, 150 MHz) δ (ppm): 183.2, 169.7, 169.6, 54.8, 42.7, 41.5, 26.9.

The same process as disclosed above for the preparation of the aminosquaramide polymer of Example 7-A was followed for the preparation of the following aminosquaramide polymers of the present invention Ex. 7-B-E having the percentages of monomers of formula A and of formula B specified in Table below, but using the amount of compounds VI, (10) and IV disclosed herein below:

| Aminosquaramide | | | | | |
|---|---|---|---|---|---|
| | Mono- | Mono- | | | |
| | mer | mer | | | |
| | A7 | A8 | | | |
| | % by | % by | Starting material | | |
| number | weight | weight | VI | 10 | IV |
| Ex. | 95 | 5 | 99.20 mg | 10.0. mg | 281.65 mg |
| 7-B | | | (0.68 mmol) | (0.04 mmol) | (0.72 mmol) |
| Ex. | 80 | 20 | 151.71 mg | 75.68 mg | 508.38 mg |
| 7-C | | | (1.04 mmol) | (0.27 mmol) | (1.31 mmol) |
| Ex. | 70 | 30 | 131.12 mg | 105.30 mg | 503.96 mg |
| 7-D | | | (190 mmol) | (0.38 mmol) | (1.28 mmol) |
| Ex. | 50 | 50 | 92.73 mg | 172.82 mg | 496.75 |
| 7-E | | | (0.64 mmol) | (0.62 mmol) | (1.26 mmol) |

### 5. Aminosquaramide Polymers Comprising 100% of One Monomer of Formula A

Example 8. Aminosquaramide polymers of Example 8 comprise aminosquaramide polymers of formula (I)-1A-1 and (I)-1B-1 wherein m is 0, X is —(CH₂)₃—, Z is CH₃, R₃ and R₄ are H, having a Mw equal to or higher 30 KDa.

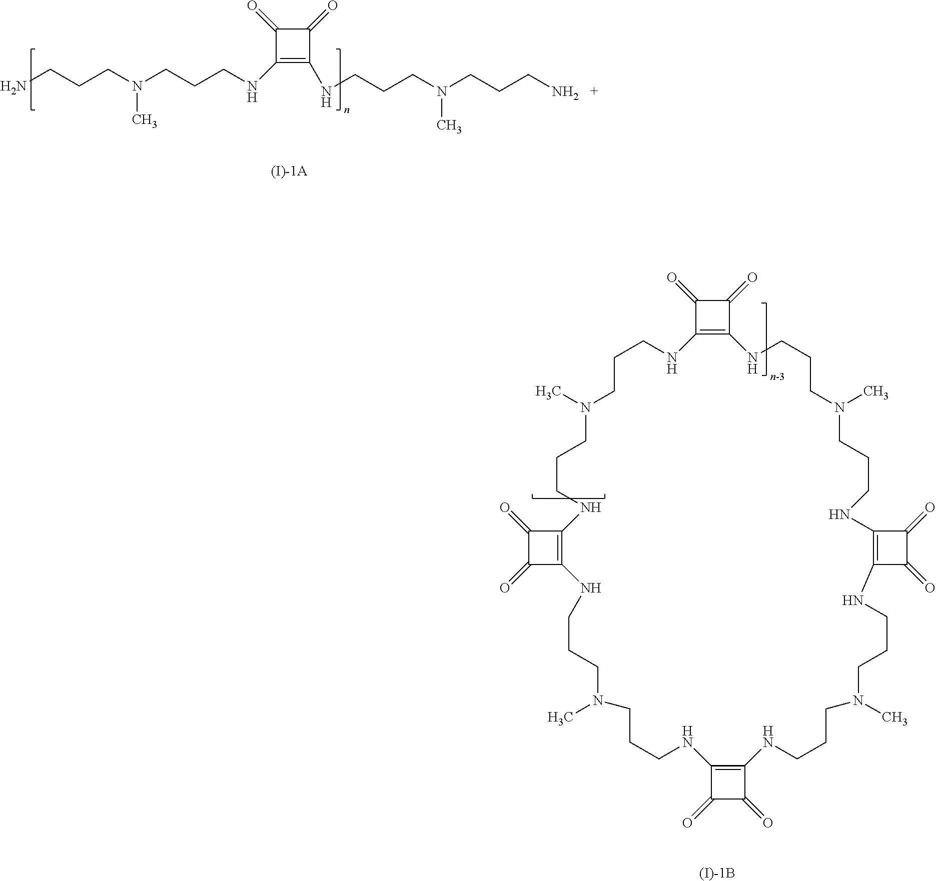

### Method D:

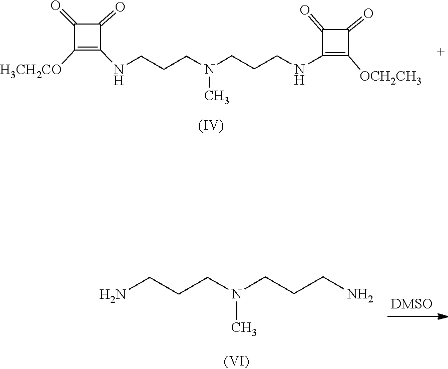

A solution of the amine (IV) (300 mg, 1.37 mmol) in 1.0 mL of DMSO was added to a stirred solution of diester (VI) (535 mg, 1.36 mmol) in 1.5 mL of DMSO at 100° C. After half an hour after the addition, the precipitation of the product makes the stirring impossible. The resulting pale-yellow solid was filtered and washed with 100 mL of ethanol, 30 mL of acetone and dried under vacuum (0.570 g). The high molecular weight (HMW) fraction was obtained after 5 cycles of filtration through 10 kDa cut off AMICON filters using a solution of formic acid 0.1% v/v in water. Fractionation and purification of a re-dissolved crude mixture solution were accomplished through a series of centrifugation steps with Amicon Ultra 15 mL centrifugal filters of 10 kDa MW cut-offs and 50 kDa, respectively (Millipore, MA, USA). Briefly, a solution of the above yellow solid (0.201 g) in 15 mL formic acid (0.3% v/v) was passed through a 10 kDa filter at 3220 G for 40 min. The filtrate solution was discharged. The concentrate was diluted with the formic acid (0.1% v/v) solution (12 mL) and spun down again. This cycle of dilution-filtration was repeated five times. Lyophilization of the isolated concentrate affords the fraction over 10 kDa as a pale-yellow solid (0.136 g, 67%). Then, a part of the isolated 10 kDa fraction (121.58 mg) was dissolved in 15 mL formic acid (0.3% v/v) and was passed through a 50 kDa filter at 3220 G for 40 min. The filtrate solution was discharged. The concentrate was diluted with the formic acid (0.1% v/v) solution (12 mL) and spun down again. This cycle of dilution-filtration was repeated five times. Lyophilization of the isolated concentrate (Example 8) affords the fraction over 10 kDa as a pale-yellow solid.

¹H-NMR spectrum of fraction over 10 kDa (D₂O/0.1% HCOOH, 600 MHz) δ (ppm): 8.21, 4.69, 3.57, 3.21, 3.14, 3.01, 2.99, 2.81, 1.99.

¹³C-NMR spectrum of fraction over 10 kDa (D₂O/0.1% HCOOH, 150 MHz) δ (ppm): 183.2, 169.3, 54.8, 42.7, 41.5, 26.9.

Three batches were performed using the process as disclosed in Example 8 and the following aminosquaramide polymers of Example 8-A/B/C were obtained. The Mw of these polymers are disclosed in Table below:

| | | yield | |
|---|---|---|---|
| Aminosquaramide | | % yield Raw | % yield polymer |
| number | Mw kDa | material | >10 KDa |
| Ex. 8-A | 51.0 | 94 | 67 |
| Ex. 8-B | 39.3 | 95 | 62 |
| Ex. 8-C | 31.5 | 89 | 57 |

### 6. Transfection Activity of the Aminosquaramide Polymers of Examples 1-8

The transfection activity of the aminosquaramide polymers of Examples 1-8 was evaluated following the transfection protocol 1 disclosed below and measuring the fluorescence in arbitrary units (following the measuring protocol 2) or measuring the transfected cell (following the measuring protocol 3).

### Transfection Protocol: Protocol 1

The same transfection protocol used for HEK293 cells with the aminosquaramide compound (I)-1 of the present invention as defined above, were used with the squaramide polymers of Examples 1-8 of the present invention. As positive control, the same transfection test was also performed with the standard commercial reagent Lipofectamine2000® (Invitrogen) including the same corresponding macromolecules to be transfected, following the manufacturer's instructions.

### Measurement of the Transfection Activity

### Protocol 2

The measurement of the transfection was performed by the fluorescence generated (expressed as arbitrary units) by the Green fluorescent protein synthetized in cultured Hela cells 48 h after the transfection of 1 μg of pmax-GFP packed with varying concentrations from 2-12 ppm of the tested polymers of the invention was performed. Fluorescence values are expressed in terms of comparison with the background detection of green fluorescence in the negative control after the cultured of Hela cells without the use of any transfection agent.

### Results

The arbitrary units of fluorescence obtained 48 h after the transfection of 1 μg of pmax-GFP packed with varying concentrations from 2-12 ppm of the aminosquaramide polymers of the present invention are disclosed in Table below:

| Aminosquaramide | Fluorescence (arbitrary units) | | | | | |
|---|---|---|---|---|---|---|
| polymer Example | 2 ppm | 4 ppm | 6 ppm | 8 ppm | 10 ppm | 12 ppm |
| Example 1 | 6111 | 26392 | 35540 | 29387 | 28031 | 22361 |
| Example 2 | 683 | 8621 | 13302 | 12554 | 14543 | 10270 |
| Example 3-B | 4281 | 20234 | 5586 | 4075 | 9616 | 9360 |
| Example 3-C | ND | 3970 | 2168 | 2786 | 158 | 863 |
| Example 4 | ND | 602 | 2504 | 969 | 209 | ND |
| Example 5 | 26232 | 31640 | 50925 | 42185 | 51899 | 34769 |
| Example 6 | 4511 | 5814 | 4920 | 9652 | 20441 | 7320 |
| Example 7-A | 8595 | 20884 | 21884 | 27145 | 27862 | 34149 |
| Example 7-C | 2376 | 17293 | 11662 | 9891 | 16357 | 29063 |

ND: fluorescence not detected

### Protocol 3

The transfection efficiency (%) was measured as a number of transfected Hella cells after 48 h of the treatment of 10⁵ cells per well with 100 ng of pmax-GFP packed with 6 ppm of the aminosquaramide polymers of Example 8. DNA packing was made in 500 μl of PBS buffer using 500 ng of pmax-GFP and 30 ppm of aminosquaramide polymer. After that, 100 μl of the packing reaction was added to each well and the efficiency was calculated from triplicates (n=3). The transfection efficiency was calculated by comparison of the total green fluorescence of each well with the maximum green fluorescence signal in arbitrary units provided by 106 sorted transfected cells obtained with a fixed spectrofluorometer gain. The gain was fixed to 1100 to give a lecture of maximum fluorescence of 260.000 a.u. corresponding at 100% of transfected cells.

### Results

The % of transfection efficiency after the transfection of 100 ng of pmax-GFP packed with 6 ppm of the aminosquaramide polymers of the present invention are disclosed in Table below:

| Aminosquaramide polymer Example | % transfection |
|---|---|
| Ex. 8-A | 66.0 ± 4.5 |
| Ex. 8-B | 85.0 ± 5.9 |

### CONCLUSION

These results show a positive transfection outcome using aminosquaramide polymers of the present invention having a huge variety of backbones and moieties (attached to it) including polymers having only monomers of formula A equal or different (random located) and polymers having monomer A and monomer B, in block or randomly distributed; having molecular weights (Mw) falling within the scope of the present invention (from 2000 to 91000). In fact, the transfection was observed for all tested concentrations (from 2-12 ppm) Therefore, these results confirmed that the aminosquaramide polymers of the present invention are appropriate for being use as transfecting agents and at the same time being detected by fluorescence.

### CITATION LIST

1. Greene and Wuts, Protecting Groups in Organic Synthesis, chapter: “Protection of amines”. Third Edition, John Wiley & Sons (1999) pages 494-653).
2. Neufeld, R. et al. “Accurate Molecular Weight Determination of Small Molecules via DOSY-NMR by Using External Calibration Curves with Normalized Diffusion Coefficients”. Chem. Sci. 2015, vol. 6 (6), pp. 3354-3364.
3. Angel Sampedro et al. Bioconjugate Chem., 2014, 25, 1537-1546
4. US2011294772
5. Rotger et al. “Efficient macrocyclization preorganized palindromic oliosquaramides”, Angewandte Chemie International Edition, Verlag Chemie, 2006, vol. 45 (41), pp. 6844-6848
6. Chasák et al. discloses (cf. Squaric acid analogues in medicinal chemistry”, European journal of Medicinal Chemistry. 2020, vol. 209.
7. Macromolecules, 2018, 51, 12, 4688-4693

For reasons of completeness, various aspects of the invention are set out in the following numbered clauses:

Clause 1. An aminosquaramide polymer comprising a backbone of repeating structural units, being these repeating structural units equal or different of formula (II) or a salt thereof,

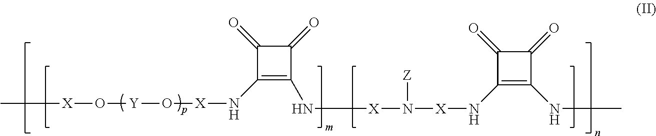

wherein:
X is selected from the group consisting of —(CH₂)_{q}—, —(CHR₁)_{q}— and —(CR₁R₂)_{q}—;
Y is selected from the group consisting of —(CH₂)ᵣ—, —(CHR₃)ᵣ— and —(CR₃R₄)ᵣ—;
R₁ and R₂ are independently selected from the group consisting of H and Z′;
R₃ and R₄ are independently selected from the group consisting of H and Z′;
each Z and Z′ are independently selected from the group consisting of an inert moiety, a detection moiety, and an active moiety;
the inert moiety is selected from the group consisting of —(C₁-C₁₂)alkyl; —(C₂-C₁₂)alkenyl; —(C₂-C₆)alkynyl; -Cy1; —(C₁-C₁₂)alkylene-Cy1; —(C₂-C₁₂)alkenylene-Cy1; —C₂-C₁₂)alkynylene-Cy1; -Cy2-(C₁-C₁₂)alkyl; -Cy2-(C₂-C₁₂)alkenyl; and -Cy2-(C₂-C₁₂)alkynyl; being Cy1 a known ring system independently selected from the group consisting of 5- to 7-membered saturated or partially unsaturated carbocyclic or heterocyclic ring; and 5- to 7-membered carbocyclic or heterocyclic aromatic ring; Cy2 a bivalent known ring system independently selected from the group consisting of 5- to 7-membered saturated or partially unsaturated carbocyclic or heterocyclic ring; and 5- to 7-membered carbocyclic or heterocyclic aromatic ring; and being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl; being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl;
R₅ and R₆ are independently selected from the group consisting of —(C₁-C₁₂)alkyl and an amine protecting group;
Halogen is selected from the group consisting of F, Cl, Br, and I;
the detection moiety is selected from the group consisting of chromophore moiety, fluorescent moiety, phosphorescent moiety, luminescent moiety, light absorbing moiety, radioactive moiety, transition metal and isotope mass tag moiety; and
the active moiety is selected from the group consisting of a pharmaceutically active ingredient, a veterinary active ingredient, and a cosmetic active ingredient;
n is an integer from 10 to 180;
m is 0 or 1;
p is an integer from 1 to 10;
q is an integer from 2 to 6; and
r is an integer from 2 to 4.

Clause 2. The aminosquaramide polymer according to clause 1, wherein:
(a) in the repeating structural units, being these repeating structural units equal or different of formula (II) or a salt thereof, m is 0 and thereby the aminosquaramide polymer is an aminosquaramide polymer (I) selected from the group consisting of:
a polymer of formula (I)-1A, or a salt thereof;

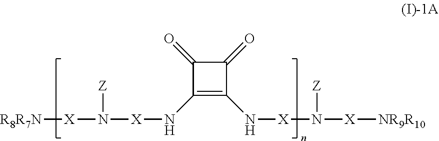

a polymer of formula (I)-1B, or a salt thereof;

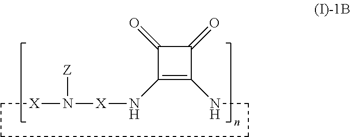

and
a mixture of one or more polymers of formula (I)-1A, or a salt thereof; and one or more polymers of formula (I)-1B, or a salt thereof;
or alternatively,
(b) in the repeating structural units, being these repeating structural units equal or different of formula (II) or a salt thereof, m is 1 and thereby the aminosquaramide polymer is an aminosquaramide polymer (I) selected from the group consisting of:
a polymer of formula (I)-2A, or a salt thereof;

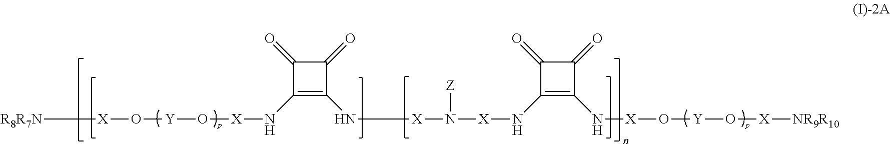

a polymer of formula (I)-2B, or a salt thereof;

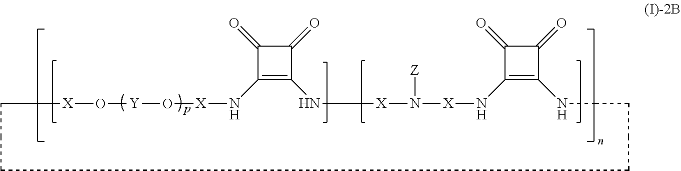

and
a mixture of one or more polymers of formula (I)-2A, or a salt thereof; and one or more polymers of formula (I)-2B, or a salt thereof;
wherein:
X, Y, Z, Z′, R₁, R₂, R₃, R₄, R₅, R₆, halogen, n, p, q, and r are as defined in clause 1; dashed line [symbol] is a single bond that bonds X and NH;
R₇, R₈, R₉ and R₁₀ are independently selected from the group consisting of H, an amine protecting group, a detection moiety, an active moiety, and a moiety of formula (III):

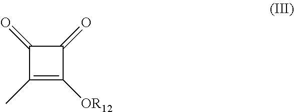

being:
the detection moiety and the active moiety as defined in clause 1;
R₁₂ is selected from the group consisting of (C₁-C₆)alkyl, H and an alcohol protecting group;
with the proviso that:
when one of R₇ and R₈ is a detection moiety or an active moiety, the other is selected from H and alcohol protecting group; and
when one of R₉ and R₁₀ is a detection moiety or an active moiety, the other is selected from H and alcohol protecting group.

Clause 3. The aminosquaramide polymer according to clauses 1 or 2, wherein in the repeating structural units, being these repeating structural units equal or different of formula (II) or a salt thereof, n is an integer from 20 to 100.

Clause 4. The aminosquaramide polymer according to any of the clauses 1-3, wherein the aminosquaramide polymer is a aminosquaramide polymer of formula (I) having a number average molecular weight (Mn) from 6000 to 30000 Da measured by Gel Permeation Chromatography-Visible Ultraviolet (GPC-UV).

Clause 5. The aminosquaramide polymer according to any of the clauses 1-4, wherein all repeating structural units are equal of formula (II) or a salt thereof.

Clause 6. The aminosquaramide polymer according to clause 5; which is:
a compound of formula (I)-1A, wherein: X is —(CH₂)_{q}—; or
a compound of formula (I)-1B, wherein: X is —(CH₂)_{q}—; or
a compound of formula (I)-2A, wherein: X is —(CH₂)_{q}—; Y is —(CH₂)ᵣ—; or
a compound of formula (I)-2B, wherein: X is —(CH₂)_{q}—; Y is —(CH₂)ᵣ—; and
being R₅, R₆, q and r as defined in clause 1;
and particularly,
a compound of formula (I)-1A, wherein: X is —(CH₂)₃—; or
a compound of formula (I)-1B, wherein: X is —(CH₂)₃—; or
a compound of formula (I)-2A, wherein: X is —(CH₂)₃—; Y is —(CH₂)₂—; R₅ and R₆ are H, and p is 2; or
a compound of formula (I)-2B, wherein: X is —(CH₂)₃—; Y is —(CH₂)₂—; R₅ and R₆ are H, and p is 2; and
being R₅ and R₆ as defined in clause 1.

Clause 7. The aminosquaramide polymer according to any of the clauses 1-4, wherein the repeating structural units of formula (II) or a salt thereof are differents.

Clause 8. The aminosquaramide polymer according to clause 7, wherein:
Z is —CH₃ in a percentage of repeating structural units from 50 to 90% in relation to the total weight of the polymer or a salt; and
Z is other than —CH₃ in a percentage of repeating structural units from 10 to 50% in relation to the total weight of the polymer or a salt.

Clause 9. The aminosquaramide polymer according to any of the clauses 7-8, which is:
a compound of formula (I)-1A, wherein: X is —(CH₂)_{q}—; or
a compound of formula (I)-1B, wherein: X is —(CH₂)_{q}—; or
a compound of formula (I)-2A, wherein: X is —(CH₂)_{q}—; Y is —(CH₂)ᵣ—; or
a compound of formula (I)-2B, wherein: X is —(CH₂)_{q}—; Y is —(CH₂)ᵣ—; and
being R₅, R₆, q and r as defined in clause 1;
and particularly,
a compound of formula (I)-1A, wherein: X is —(CH₂)₃—; or
a compound of formula (I)-1B, wherein: X is —(CH₂)₃—; or
a compound of formula (I)-2A, wherein: X is —(CH₂)₃—; Y is —(CH₂)₂—; R₅ and R₆ are H, and p is 2; or
a compound of formula (I)-2B, wherein: X is —(CH₂)₃—; Y is —(CH₂)₂—; R₅ and R₆ are H, and p is 2; and
being R₅ and R₆ as defined in clause 1.

Clause 10. A conjugate comprising the polymer as defined in any of the clauses 1-9, and one or more molecules of interest; particularly the molecule of interest is selected from the group consisting of an active ingredient, an amino acid-containing compound, a nucleic acid-containing compound, and a mixture thereof.

Clause 11. A composition comprising the polymer as defined in any of the clauses 1-9; or alternatively, the conjugate as defined in clause 10, together with one or more appropriate excipients or carriers.

Clause 12. Use of the polymer as defined in any of the clauses 1-9 wherein Z and Z′ are inert moieties; or alternatively a composition as defined in clause 11 containing it, as a carrier;

Clause 13. A polymer as defined in any of the clauses 1-9, wherein at least one of Z and Z′ is a pharmaceutically or veterinary active moiety, or alternatively, a conjugate as defined in clause 10 containing it, or alternatively, a composition as defined in clause 11 containing the polymer or the conjugate, for use in therapy;
or alternatively, a polymer as defined in any of the clauses 1-9, wherein at least one of Z and Z′ is a detection moiety; or alternatively, a conjugate as defined in clause 10 containing it, or alternatively, a composition as defined in clause 11 containing the polymer or the conjugate, for use in diagnostic;
or alternatively, use in cosmetics of a polymer as defined in any of the clauses 1-9, wherein at least one of Z and Z′ is a cosmetically active moiety; or alternatively, a conjugate as defined in clause 10 containing it, or alternatively, a composition as defined in clause 11 containing the polymer or the conjugate,

Clause 14. A conjugate as defined in clause 10; wherein the molecule of interest is at least a molecule selected from the group consisting of a pharmaceutically or veterinary active ingredient, an amino acid-containing compound, a nucleic acid-containing compound, or a mixture thereof, for use in therapy;
or alternatively, a conjugate as defined in clause 10; wherein the molecule of interest is at least a nucleic acid-containing compound, or alternatively a composition as defined in clause 11 containing it, for use as a transfecting agent;
or alternatively, the use in cosmetics of the conjugate as defined in clause 10, wherein the molecule of interest is at least one cosmetically active ingredient.

Clause 15. A kit comprising:
the polymer as defined in any of the clauses 1-9; or alternatively the composition as defined in clause 11 containing the polymer; optionally means to prepare the conjugate, and optionally means to administrate the conjugate;
or alternatively,
the conjugate as defined in clause 10; or alternatively the composition as defined in clause 11 containing the conjugate, and optionally means to administrate the conjugate.

1. An aminosquaramide polymer comprising a backbone of repeating structural units, being these repeating structural units equal or different and selected from the group consisting of:
a monomer of formula A, or a salt thereof; 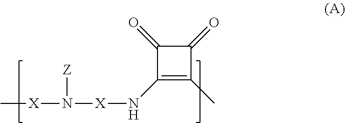
and a monomer of formula B, or a salt thereof 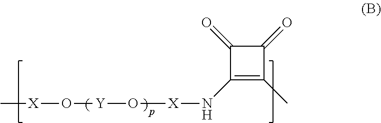
wherein:
X is selected from the group consisting of —(CH₂)_{q}—, —(CHR₁)_{q}— and —(CR₁R₂)_{q}—;
Y is selected from the group consisting of —(CH₂)ᵣ—, —(CHR₃)ᵣ— and —(CR₃R₄)ᵣ—;
R₁ and R₂ are independently selected from the group consisting of H and Z′;
R₃ and R₄ are independently selected from the group consisting of H and Z′;
each Z and Z′ are independently selected from the group consisting of an inert moiety, a detection moiety, and an active moiety;
the inert moiety is selected from the group consisting of —(C₁-C₁₂)alkyl; —(C₂-C₁₂)alkenyl; —(C₂-C₁₂)alkylene-O—(Y—O)_{w}—(C₁-C₁₂)alquil, —(C₂-C₁₂)alkylene-O—(Y—O)_{w}—(C₂-C₁₂)alkylene-NR₁₁R₁₃, —(C₂-C₆)alkynyl; -Cy1; —(C₁-C₁₂)alkylene-Cy1; —(C₂-C₁₂)alkenylene-Cy1; —(C₂-C₁₂)alkynylene-Cy1; -Cy2-(C₁-C₁₂)alkyl; -Cy2-(C₂-C₁₂)alkenyl;
and -Cy2-(C₂-C₁₂)alkynyl; being Cy1 a known ring system independently selected from the group consisting of 5- to 7-membered saturated or partially unsaturated carbocyclic or heterocyclic ring; and 5- to 7-membered carbocyclic or heterocyclic aromatic ring; Cy2 a bivalent known ring system independently selected from the group consisting of 5- to 7-membered saturated or partially unsaturated carbocyclic or heterocyclic ring; and
5- to 7-membered carbocyclic or heterocyclic aromatic ring; and being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl; being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl;
R₅ and R₆ are independently selected from the group consisting of —(C₁-C₁₂)alkyl and an amine protecting group;
R₁₁ and R₁₃ are independently selected from the group consisting of H, —(C₁-C₁₂)alkyl and an amine protecting group;
Halogen is selected from the group consisting of F, Cl, Br, and I;
the detection moiety is selected from the group consisting of chromophore moiety, fluorescent moiety, phosphorescent moiety, luminescent moiety, light absorbing moiety, radioactive moiety, transition metal and isotope mass tag moiety; and
the active moiety is selected from the group consisting of a pharmaceutically active ingredient, a veterinary active ingredient, and a cosmetic active ingredient;
p is an integer from 1 to 10;
q is an integer from 2 to 6;
r is an integer from 2 to 4;
the percentage by weight of the monomer of formula A, or a salt thereof, is from 30% to 100%;
the percentage by weight of the monomer of formula B, or a salt thereof, is from 0% to 70%;
being the sum of the percentages equal to 100%; and
the aminosquaramide polymer has a weight average molecular weight (Mw) from 2000 to 91000 Da;
with the proviso that when the percentage by weight of monomer A is 100%, then the weight average molecular weight (Mw)) of the polymer is from 4500 to 91000 Da;
being:
the weight average molecular weight (Mw) from 2000 to 30000 Da measured by Gel Permeation Chromatography-Visible Ultraviolet (GPC-UV); and
the weight average molecular weight (Mw) from higher than 30000 to 91000 Da measured by analytical size-exclusion chromatography coupled with multi-angle light scattering and refractive index detector (SEC/MALS/RI).

2. The aminosquaramide polymer according to claim 1, wherein the repeating structural units are equal or different of formula (II) or a salt thereof, 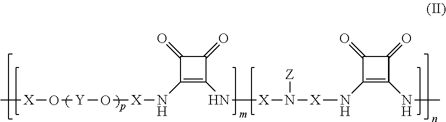
wherein:
m is 0 or 1;
with the proviso that;
when m is 0, then the % by weight of the monomer of formula A, or a salt thereof, is 100% and n is an integer from 20 to 400; and
when m is 1, then the percentage of formula A, or a salt thereof, is 50%, and the percentage of formula B or a salt thereof is 50% and n is an integer from 10 to 400.

3. The aminosquaramide polymer according to claim 2, where:
(a) in the repeating structural units, being these repeating structural units equal or different of formula (II) or a salt thereof, m is 0 and thereby the aminosquaramide polymer is an aminosquaramide polymer (I) selected from the group consisting of:
a polymer of formula (I)-1A, or a salt thereof; 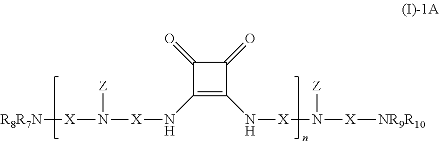
a polymer of formula (I)-1B, or a salt thereof; 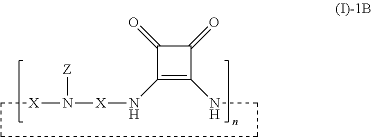
and
a mixture of one or more polymers of formula (I)-1A, or a salt thereof; and one or more polymers of formula (I)-1B, or a salt thereof;
or alternatively,
(b) in the repeating structural units, being these repeating structural units equal or different of formula (II) or a salt thereof, m is 1 and thereby the aminosquaramide polymer is an aminosquaramide polymer (I) selected from the group consisting of:
a polymer of formula (I)-2A, or a salt thereof; 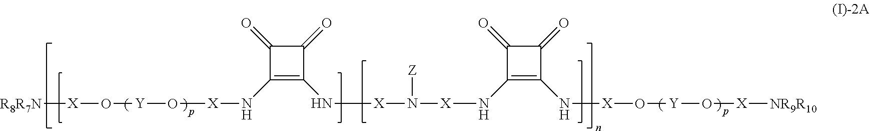
a polymer of formula (I)-2B, or a salt thereof; 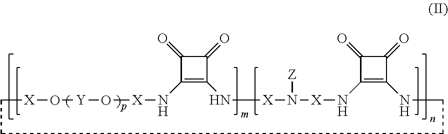
and
a mixture of one or more polymers of formula (I)-2A, or a salt thereof; and one or more polymers of formula (I)-2B, or a salt thereof;
wherein:
X, Y, Z, Z′, R₁, R₂, R₃, R₄, R₅, R₆, halogen, n, p, q, and r are as defined in claim 1;
dashed line [symbol] is a single bond that bonds X and NH;
R₇, R₈, R₉ and R₁₀ are independently selected from the group consisting of H, an amine protecting group, a detection moiety, an active moiety, and a moiety of formula (III): 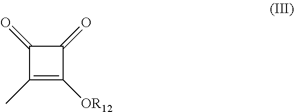
being:
the detection moiety and the active moiety as defined in claim 1;
R₁₂ is selected from the group consisting of (C₁-C₆)alkyl, H and an alcohol protecting group;
with the proviso that:
when one of R₇ and R₈ is a detection moiety or an active moiety, the other is selected from H and alcohol protecting group; and
when one of R₉ and R₁₀ is a detection moiety or an active moiety, the other is selected from H and alcohol protecting group.

4. The aminosquaramide polymer according to any of the claims 2-3, wherein all repeating structural units are equal of formula (II) or a salt thereof.

5. The aminosquaramide polymer according to claim 4; which is:
a compound of formula (I)-1A, wherein: X is —(CH₂)_{q}—; or
a compound of formula (I)-1B, wherein: X is —(CH₂)_{q}—; or
a compound of formula (I)-2A, wherein: X is —(CH₂)_{q}—; Y is —(CH₂)ᵣ—; or
a compound of formula (I)-2B, wherein: X is —(CH₂)_{q}—; Y is —(CH₂)ᵣ—; and
being R₅, R₆, q and r as defined in claim 1;
and particularly,
a compound of formula (I)-1A, wherein: X is —(CH₂)₃—; or
a compound of formula (I)-1B, wherein: X is —(CH₂)₃—; or
a compound of formula (I)-2A, wherein: X is —(CH₂)₃—; Y is —(CH₂)₂—; R₅ and R₆ are H, and p is 2; or
a compound of formula (I)-2B, wherein: X is —(CH₂)₃—; Y is —(CH₂)₂—; R₅ and R₆ are H, and p is 2; and
being R₅ and R₆ as defined in claim 1.

6. The aminosquaramide polymer according to any of the claims 2-3, wherein the repeating structural units of formula (II) or a salt thereof are different.

7. The aminosquaramide polymer according to claim 6, wherein:
Z is —CH₃ in a percentage of repeating structural units from 50 to 90% in relation to the total weight of the polymer or a salt; and
Z is other than —CH₃ in a percentage of repeating structural units from 10 to 50% in relation to the total weight of the polymer or a salt.

8. The aminosquaramide polymer according to any of the claims 6-7 which is:
a compound of formula (I)-1A, wherein: X is —(CH₂)_{q}—; or
a compound of formula (I)-1B, wherein: X is —(CH₂)_{q}—; or
a compound of formula (I)-2A, wherein: X is —(CH₂)_{q}—; Y is —(CH₂)ᵣ—; or
a compound of formula (I)-2B, wherein: X is —(CH₂)_{q}—; Y is —(CH₂)ᵣ—; and
being R₅, R₆, q and r as defined in claim 1;
and particularly,
a compound of formula (I)-1A, wherein: X is —(CH₂)₃—; or
a compound of formula (I)-1B, wherein: X is —(CH₂)₃—; or
a compound of formula (I)-2A, wherein: X is —(CH₂)₃—; Y is —(CH₂)₂—; R₅ and R₆ are H, and p is 2; or
a compound of formula (I)-2B, wherein: X is —(CH₂)₃—; Y is —(CH₂)₂—; R₅ and R₆ are H, and p is 2; and
being R₅ and R₆ as defined in claim 1.

9. The aminosquaramide polymer according to any of the claims 2-8, wherein the repeating structural units are equal or different of formula (II) or a salt thereof, and n is an integer from 20 to 400.

10. The aminosquaramide polymer according to any of the claims 2-9, wherein the repeating structural units are equal or different of formula (II) or a salt thereof, and n is an integer from 20 to 230.

11. The aminosquaramide polymer according to any of the claims 2-10, wherein the repeating structural units are equal or different of formula (II) or a salt thereof, and n is an integer from 20 to 180.

12. The aminosquaramide polymer according to any of the claims 2-11, wherein the repeating structural units are equal or different of formula (II) or a salt thereof, and n is an integer from 20 to 133.

13. The aminosquaramide polymer according to any of the claims 2-12, wherein the repeating structural units are equal or different of formula (II) or a salt thereof, and n is an integer from 26 to 133.

14. The aminosquaramide polymer according to any of the claims 2-12, wherein the repeating structural units are equal or different of formula (II) or a salt thereof, and n is an integer from 20 to 100.

15. The aminosquaramide polymer according to any of the claims 1-10, wherein the aminosquaramide polymer has a weight average molecular weight (Mw) from 5000 to 70000 Da; particularly from 5000 to 50000 Da.

16. The aminosquaramide polymer according to any of the claims 1-13, wherein the aminosquaramide polymer has a weight average molecular weight (Mw) from 5000 to 30000 Da.

17. The aminosquaramide polymer according to any of the claims 1-13, wherein the aminosquaramide polymer has a weight average molecular weight (Mw) from 6000 to 30000 Da.

18. The aminosquaramide polymer according to any of the claims 1-14, wherein the aminosquaramide polymer has a weight average molecular weight (Mw) from 6000 to 20000 Da.

19. The aminosquaramide polymer according to claim 1, wherein:
the percentage of monomer of formula B, or a salt thereof, is different than zero, and
the monomers of formula A, or a salt thereof, and of formula B, or a salt thereof, are randomly distributed.

20. The aminosquaramide polymer according to claim 19, wherein:
the percentage of monomer of formula B, or a salt thereof, is different than zero; and
the percentage by weight of the monomer of formula A, or a salt thereof, is equal to or higher than 80%;
being the sum equal to 100%.

21. The aminosquaramide polymer according to any of the claims 19-20, wherein:
The percentage of monomer of formula B, or a salt thereof, is different than zero; and
the percentage by weight of the monomer of formula A, or a salt thereof, is equal to or higher than 90%;
being the sum equal to 100%.

22. The aminosquaramide polymer according to any of the claims 19-21, wherein:
the percentage of monomer of formula B, or a salt thereof, is different than zero; and
the percentage by weight of the monomer of formula A, or a salt thereof, is equal to or higher than 95%;
being the sum equal to 100%.

23. A conjugate comprising an aminosquaramide polymer comprising a backbone of repeating structural units, being these repeating structural units equal or different and selected from the group consisting of:
a monomer of formula A, or a salt thereof; 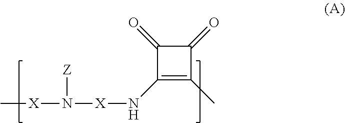
and a monomer of formula B, or a salt thereof; 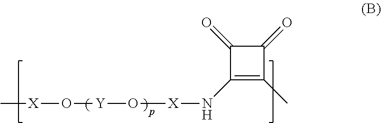
wherein:
X is selected from the group consisting of —(CH₂)_{q}—, —(CHR₁)_{q}— and —(CR₁R₂)_{q}—;
Y is selected from the group consisting of —(CH₂)ᵣ—, —(CHR₃)ᵣ— and —(CR₃R₄)ᵣ—;
R₁ and R₂ are independently selected from the group consisting of H and Z′;
R₃ and R₄ are independently selected from the group consisting of H and Z′;
each Z and Z′ are independently selected from the group consisting of an inert moiety, a detection moiety, and
an active moiety;
the inert moiety is selected from the group consisting of —(C₁-C₁₂)alkyl; —(C₂-C₁₂)alkenyl; —(C₂-C₁₂)alkylene-O—(Y—O)_{w}—(C₁-C₁₂)alquil, —(C₂-C₁₂)alkylene-O—(Y—O)—(C₂-C₁₂)alkylene-NR₁₁R₁₃, —(C₂-C₆)alkynyl; -Cy1; —(C₁-C₁₂)alkylene-Cy1; —(C₂-C₁₂)alkenylene-Cy1; —(C₂-C₁₂)alkynylene-Cy1; -Cy2-(C₁-C₁₂)alkyl; -Cy2-(C₂-C₁₂)alkenyl;
and -Cy2-(C₂-C₁₂)alkynyl; being Cy1 a known ring system independently selected from the group consisting of 5- to 7-membered saturated or partially unsaturated carbocyclic or heterocyclic ring; and 5- to 7-membered carbocyclic or heterocyclic aromatic ring; Cy2 a bivalent known ring system independently selected from the group consisting of 5- to 7-membered saturated or partially unsaturated carbocyclic or heterocyclic ring; and
5- to 7-membered carbocyclic or heterocyclic aromatic ring; and being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl; being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl;
R₅ and R₆ are independently selected from the group consisting of —(C₁-C₁₂)alkyl and an amine protecting group;
R₁₁ and R₁₃ are independently selected from the group consisting of H, —(C₁-C₁₂)alkyl and an amine protecting group;
Halogen is selected from the group consisting of F, Cl, Br, and I;
the detection moiety is selected from the group consisting of chromophore moiety, fluorescent moiety, phosphorescent moiety, luminescent moiety, light absorbing moiety, radioactive moiety, transition metal and isotope mass tag moiety; and
the active moiety is selected from the group consisting of a pharmaceutically active ingredient, a veterinary active ingredient, and a cosmetic active ingredient;
p is an integer from 1 to 10;
q is an integer from 2 to 6;
r is an integer from 2 to 4;
the percentage by weight of the monomer of formula A or a salt thereof is from 30% to 100% by weight;
the percentage by weight of the monomer of formula B or a salt thereof is from 0% to 70% by weight;
being the sum of the percentages equal to 100%; and
the aminosquaramide polymer has a weight average molecular weight (Mw) from 2000 to 91000 Da;
and one or more molecules of interest;
being:
the weight average molecular weight (Mw) from 2000 to 30000 Da measured by Gel Permeation Chromatography-Visible Ultraviolet (GPC-UV); and
the weight average molecular weight (Mw) from higher than 30000 to 91000 Da measured by analytical size-exclusion chromatography coupled with multi-angle light scattering and refractive index detector (SEC/MALS/RI).

24. The conjugate according to claim 23, wherein the aminosquaramide polymer is one comprising repeating structural units equal or different of formula (II) or a salt thereof, 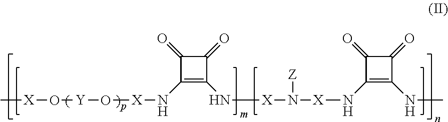
wherein:
m is 0 or 1;
n is an integer from 10 to 400.

25. The conjugate according to claim 24, wherein the aminosquaramide polymer is one as defined in any of the claims 1-22.

26. The conjugate according to any of the claims 23-25, wherein the molecule of interest is selected from the group consisting of an active ingredient, an amino acid-containing compound, a nucleic acid-containing compound, and a mixture thereof.

27. A composition comprising an aminosquaramide polymer comprising a backbone of repeating structural units, being these repeating structural units equal or different and selected from the group consisting of:
a monomer of formula A, or a salt thereof 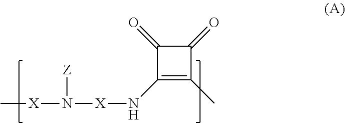
and a monomer of formula B, or a salt thereof 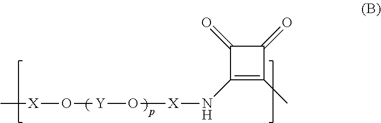
wherein:
X is selected from the group consisting of —(CH₂)_{q}—, —(CHR₁)_{q}— and —(CR₁R₂)_{q}—;
Y is selected from the group consisting of —(CH₂)ᵣ—, —(CHR₃)ᵣ— and —(CR₃R₄)ᵣ—;
R₁ and R₂ are independently selected from the group consisting of H and Z′;
R₃ and R₄ are independently selected from the group consisting of H and Z′;
each Z and Z′ are independently selected from the group consisting of an inert moiety, a detection moiety, and an active moiety;
the inert moiety is selected from the group consisting of —(C₁-C₁₂)alkyl; —(C₂-C₁₂)alkenyl; —(C₂-C₁₂)alkylene-O—(Y—O)_{w}—(C₁-C₁₂)alquil, —(C₂-C₁₂)alkylene-O—(Y—O)—(C₂-C₁₂)alkylene-NR₁₁R₁₃, —(C₂-C₆)alkynyl; -Cy1; —(C₁-C₁₂)alkylene-Cy1; —(C₂-C₁₂)alkenylene-Cy1; —(C₂-C₁₂)alkynylene-Cy1; -Cy2-(C₁-C₁₂)alkyl; -Cy2-(C₂-C₁₂)alkenyl;
and -Cy2-(C₂-C₁₂)alkynyl; being Cy1 a known ring system independently selected from the group consisting of 5- to 7-membered saturated or partially unsaturated carbocyclic or heterocyclic ring; and 5- to 7-membered carbocyclic or heterocyclic aromatic ring; Cy2 a bivalent known ring system independently selected from the group consisting of 5- to 7-membered saturated or partially unsaturated carbocyclic or heterocyclic ring; and
5- to 7-membered carbocyclic or heterocyclic aromatic ring; and being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl; being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl;
R₅ and R₆ are independently selected from the group consisting of —(C₁-C₁₂)alkyl and an amine protecting group;
R₁₁ and R₁₃ are independently selected from the group consisting of H, —(C₁-C₁₂)alkyl and an amine protecting group;
Halogen is selected from the group consisting of F, Cl, Br, and I;
the detection moiety is selected from the group consisting of chromophore moiety, fluorescent moiety, phosphorescent moiety, luminescent moiety, light absorbing moiety, radioactive moiety, transition metal and isotope mass tag moiety; and
the active moiety is selected from the group consisting of a pharmaceutically active ingredient, a veterinary active ingredient, and a cosmetic active ingredient;
p is an integer from 1 to 10;
q is an integer from 2 to 6;
r is an integer from 2 to 4;
the percentage by weight of the monomer of formula A, or a salt thereof is from 30% to 100% by weight;
the percentage by weight of the monomer of formula B, or a salt thereof is from 0% to 70% by weight;
being the sum of the percentages equal to 100%; and
the aminosquaramide polymer has a weight average molecular weight (Mw) from 2000 to 91000 Da;
being:
the weight average molecular weight (Mw) from 2000 to 30000 Da measured by Gel Permeation Chromatography-Visible Ultraviolet (GPC-UV); and
the weight average molecular weight (Mw) from higher than 30000 to 91000 Da measured by analytical size-exclusion chromatography coupled with multi-angle light scattering and refractive index detector (SEC/MALS/RI);
or alternatively, the conjugate as defined in any of the claims 23-26,
together with one or more appropriate excipients or carriers.

28. The composition according to claim 27, wherein the aminosquaramide polymer is one comprising repeating structural units equal or different of formula (II) or a salt thereof, 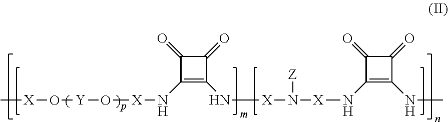
wherein:
m is 0 or 1; and
n is an integer from 10 to 400.

29. The composition according to any of the claims 27-28, wherein the aminosquaramide polymer is as defined in any of the claims 1-22.

30. Use of an aminosquaramide polymer comprising a backbone of repeating structural units, being these repeating structural units equal or different and selected from the group consisting of:
a monomer of formula A, or a salt thereof 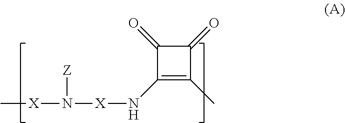
and a monomer of formula B, or a salt thereof 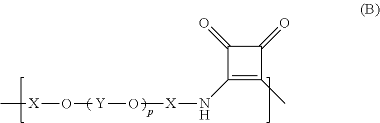
wherein:
X is selected from the group consisting of —(CH₂)_{q}—, (CHR₁)_{q}— and —(CR₁R₂)_{q}—;
Y is selected from the group consisting of —(CH₂)ᵣ—, —(CHR₃)ᵣ— and —(CR₃R₄)ᵣ—;
R₁ and R₂ are independently selected from the group consisting of H and Z′;
R₃ and R₄ are independently selected from the group consisting of H and Z′;
each Z and Z′ are independently selected from the group consisting of an inert moiety, a detection moiety, and an active moiety;
the inert moiety is selected from the group consisting of —(C₁-C₁₂)alkyl; —(C₂-C₁₂)alkenyl; —(C₂-C₁₂)alkylene-O—(Y—O)_{w}—(C₁-C₁₂)alquil, —(C₂-C₁₂)alkylene-O—(Y—O)—(C₂-C₁₂)alkylene-NR₁₁R₁₃, —(C₂-C₆)alkynyl; -Cy1; —(C₁-C₁₂)alkylene-Cy1; —(C₂-C₁₂)alkenylene-Cy1; —(C₂-C₁₂)alkynylene-Cy1; -Cy2-(C₁-C₁₂)alkyl; -Cy2-(C₂-C₁₂)alkenyl;
and -Cy2-(C₂-C₁₂)alkynyl; being Cy1 a known ring system independently selected from the group consisting of 5- to 7-membered saturated or partially unsaturated carbocyclic or heterocyclic ring; and 5- to 7-membered carbocyclic or heterocyclic aromatic ring; Cy2 a bivalent known ring system independently selected from the group consisting of 5- to 7-membered saturated or partially unsaturated carbocyclic or heterocyclic ring; and
5- to 7-membered carbocyclic or heterocyclic aromatic ring; and being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl; being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl;
R₅ and R₆ are independently selected from the group consisting of —(C₁-C₁₂)alkyl and an amine protecting group;
R₁₁ and R₁₃ are independently selected from the group consisting of H, —(C₁-C₁₂)alkyl and an amine protecting group;
Halogen is selected from the group consisting of F, Cl, Br, and I;
the detection moiety is selected from the group consisting of chromophore moiety, fluorescent moiety, phosphorescent moiety, luminescent moiety, light absorbing moiety, radioactive moiety, transition metal and isotope mass tag moiety; and
the active moiety is selected from the group consisting of a pharmaceutically active ingredient, a veterinary active ingredient, and a cosmetic active ingredient;
p is an integer from 1 to 10;
q is an integer from 2 to 6;
r is an integer from 2 to 4;
the percentage by weight of the monomer of formula A, or a salt thereof is from 30% to 100% by weight;
the percentage by weight of the monomer of formula B, or a salt thereof is from 0% to 70% by weight;
being the sum of the percentages equal to 100%; and
the aminosquaramide polymer has a weight average molecular weight (Mw) from 2000 to 91000 Da;
being:
the weight average molecular weight (Mw) from 2000 to 30000 Da measured by Gel Permeation Chromatography-Visible Ultraviolet (GPC-UV); and
the weight average molecular weight (Mw) from higher than 30000 to 91000 Da measured by analytical size-exclusion chromatography coupled with multi-angle light scattering and refractive index detector (SEC/MALS/RI);
wherein Z and Z′ are inert moieties;
or alternatively a composition as defined in any of the claims 27-30 containing it, as a carrier.

31. The use of the aminosquaramide polymer according to claim 30, wherein the aminosquaramide polymer is one comprising repeating structural units equal or different of formula (II) or a salt thereof, 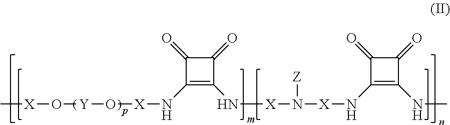
wherein:
m is 0 or 1; and
n is an integer from 10 to 400.

32. The composition according to any of the claims 27-29, wherein the aminosquaramide polymer is as defined in any of the claims 1-22.

33. A polymer selected from the group consisting of an aminosquaramide polymer comprising a backbone of repeating structural units, being these repeating structural units equal or different and selected from the group consisting of:
a monomer of formula A, or a salt thereof; 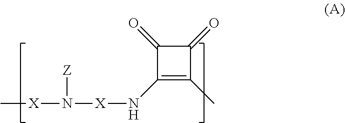
and a monomer of formula B, or a salt thereof; 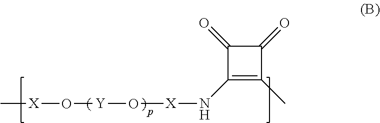
wherein:
X is selected from the group consisting of —(CH₂)_{q}— —(CHR₁)_{q} and —(CR₁R₂)_{q}—;
Y is selected from the group consisting of —(CH₂)ᵣ—, —(CHR₃)ᵣ— and —(CR₃R₄)ᵣ—;
R₁ and R₂ are independently selected from the group consisting of H and Z′;
R₃ and R₄ are independently selected from the group consisting of H and Z′;
each Z and Z′ are independently selected from the group consisting of an inert moiety, a detection moiety, and an active moiety;
the inert moiety is selected from the group consisting of —(C₁-C₁₂)alkyl; —(C₂-C₁₂)alkenyl; —(C₂-C₁₂)alkylene-O—(Y—O)_{w}—(C₁-C₁₂)alquil, —(C₂-C₁₂)alkylene-O—(Y—O)_{w}—(C₂-C₁₂)alkylene-NR₁₁R₁₃, —(C₂-C₆)alkynyl; -Cy1; —(C₁-C₁₂)alkylene-Cy1; —(C₂-C₁₂)alkenylene-Cy1; —(C₂-C₁₂)alkynylene-Cy1; -Cy2-(C₁-C₁₂)alkyl; -Cy2-(C₂-C₁₂)alkenyl;
and -Cy2-(C₂-C₁₂)alkynyl; being Cy1 a known ring system independently selected from the group consisting of 5- to 7-membered saturated or partially unsaturated carbocyclic or heterocyclic ring; and 5- to 7-membered carbocyclic or heterocyclic aromatic ring; Cy2 a bivalent known ring system independently selected from the group consisting of 5- to 7-membered saturated or partially unsaturated carbocyclic or heterocyclic ring; and
5- to 7-membered carbocyclic or heterocyclic aromatic ring; and being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl; being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl;
R₅ and R₆ are independently selected from the group consisting of —(C₁-C₁₂)alkyl and an amine protecting group;
R₁₁ and R₁₃ are independently selected from the group consisting of H, —(C₁-C₁₂)alkyl and an amine protecting group;
Halogen is selected from the group consisting of F, Cl, Br, and I;
the detection moiety is selected from the group consisting of chromophore moiety, fluorescent moiety, phosphorescent moiety, luminescent moiety, light absorbing moiety, radioactive moiety, transition metal and isotope mass tag moiety; and
the active moiety is selected from the group consisting of a pharmaceutically active ingredient, a veterinary active ingredient, and a cosmetic active ingredient;
p is an integer from 1 to 10;
q is an integer from 2 to 6;
r is an integer from 2 to 4;
the percentage by weight of the monomer of formula A or a salt thereof is from 30% to 100% by weight;
the percentage by weight of the monomer of formula B or a salt thereof is from 0% to 70% by weight;
being the sum of the percentages equal to 100%; and
the aminosquaramide polymer has a weight average molecular weight (Mw) from 2000 to 91000 Da;
being:
the weight average molecular weight (Mw) from 2000 to 30000 Da measured by Gel Permeation Chromatography-Visible Ultraviolet (GPC-UV); and
the weight average molecular weight (Mw) from higher than 30000 to 91000 Da measured by analytical size-exclusion chromatography coupled with multi-angle light scattering and refractive index detector (SEC/MALS/RI);
an aminosquaramide polymer comprising repeating structural units equal or different of formula (II) or a salt thereof, 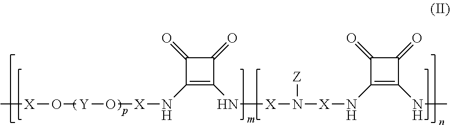
wherein:
m is 0 or 1; and
n is an integer from 10 to 400;
and a polymer as defined in any of the claims 1-22,
wherein at least one of Z and Z′ is a pharmaceutically or veterinary active moiety,
or alternatively, a conjugate as defined in any of the claims 23-26 containing it,
or alternatively, a composition as defined in any of the claims 27-29 containing the polymer or the conjugate, for use in therapy.

34. A polymer selected from the group consisting of an aminosquaramide polymer comprising a backbone of repeating structural units, being these repeating structural units equal or different and selected from the group consisting of:
a monomer of formula A, or a salt thereof; 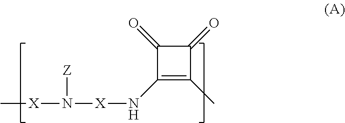
and a monomer of formula B, or a salt thereof; 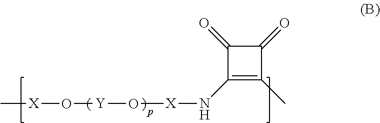
wherein:
X is selected from the group consisting of —(CH₂)_{q}—, —(CHR₁)_{q}— and —(CR₁R₂)_{q}—;
Y is selected from the group consisting of —(CH₂)ᵣ—, —(CHR₃)ᵣ— and —(CR₃R₄)ᵣ—;
R₁ and R₂ are independently selected from the group consisting of H and Z′;
R₃ and R₄ are independently selected from the group consisting of H and Z′;
each Z and Z′ are independently selected from the group consisting of an inert moiety, a detection moiety, and an active moiety;
the inert moiety is selected from the group consisting of —(C₁-C₁₂)alkyl; —(C₂-C₁₂)alkenyl; —(C₂-C₁₂)alkylene-O—(Y—O)_{w}—(C₁-C₁₂)alquil, —(C₂-C₁₂)alkylene-O—(Y—O)—(C₂-C₁₂)alkylene-NR₁₁R₁₃, —(C₂-C₆)alkynyl; -Cy1; —(C₁-C₁₂)alkylene-Cy1; —(C₂-C₁₂)alkenylene-Cy1; —(C₂-C₁₂)alkynylene-Cy1; -Cy2-(C₁-C₁₂)alkyl; -Cy2-(C₂-C₁₂)alkenyl;
and -Cy2-(C₂-C₁₂)alkynyl; being Cy1 a known ring system independently selected from the group consisting of 5- to 7-membered saturated or partially unsaturated carbocyclic or heterocyclic ring; and 5- to 7-membered carbocyclic or heterocyclic aromatic ring; Cy2 a bivalent known ring system independently selected from the group consisting of 5- to 7-membered saturated or partially unsaturated carbocyclic or heterocyclic ring; and
5- to 7-membered carbocyclic or heterocyclic aromatic ring; and being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl; being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl;
R₅ and R₆ are independently selected from the group consisting of —(C₁-C₁₂)alkyl and an amine protecting group;
R₁₁ and R₁₃ are independently selected from the group consisting of H, —(C₁-C₁₂)alkyl and an amine protecting group;
Halogen is selected from the group consisting of F, Cl, Br, and I;
the detection moiety is selected from the group consisting of chromophore moiety, fluorescent moiety, phosphorescent moiety, luminescent moiety, light absorbing moiety, radioactive moiety, transition metal and isotope mass tag moiety; and
the active moiety is selected from the group consisting of a pharmaceutically active ingredient, a veterinary active ingredient, and a cosmetic active ingredient;
p is an integer from 1 to 10;
q is an integer from 2 to 6;
r is an integer from 2 to 4;
the percentage by weight of the monomer of formula A or a salt thereof is from 30% to 100% by weight;
the percentage by weight of the monomer of formula B or a salt thereof is from 0% to 70% by weight;
being the sum of the percentages equal to 100%; and
the aminosquaramide polymer has a weight average molecular weight (Mw) from 2000 to 91000 Da;
being:
the weight average molecular weight (Mw) from 2000 to 30000 Da measured by Gel Permeation Chromatography-Visible Ultraviolet (GPC-UV); and
the weight average molecular weight (Mw) from higher than 30000 to 91000 Da measured by analytical size-exclusion chromatography coupled with multi-angle light scattering and refractive index detector (SEC/MALS/RI);
an aminosquaramide polymer comprising repeating structural units equal or different of formula (II) or a salt thereof, 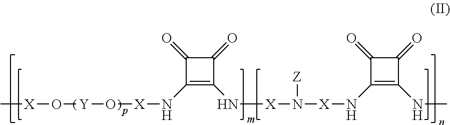
wherein:
m is 0 or 1; and
n is an integer from 10 to 400;
and a polymer as defined in any of the claims 1-22,
wherein at least one of Z and Z′ is a detection moiety;
or alternatively, a conjugate as defined in any of the claims 23-26 containing it, or alternatively, a composition as defined in any of the claims 27-29 containing the polymer or the conjugate, for use in diagnostic

35. Use in cosmetics of a polymer selected from the group consisting of aminosquaramide polymer comprising a backbone of repeating structural units, being these repeating structural units equal or different and selected from the group consisting of:
a monomer of formula A, or a salt thereof; 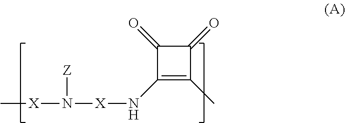
and a monomer of formula B, or a salt thereof; 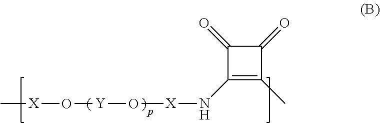
wherein:
X is selected from the group consisting of —(CH₂)_{q}—, —(CHR₁)_{q}— and —(CR₁R₂)_{q}—;
Y is selected from the group consisting of —(CH₂)ᵣ—, —(CHR₃)ᵣ— and —(CR₃R₄)ᵣ—;
R₁ and R₂ are independently selected from the group consisting of H and Z′;
R₃ and R₄ are independently selected from the group consisting of H and Z′;
each Z and Z′ are independently selected from the group consisting of an inert moiety, a detection moiety, and an active moiety;
the inert moiety is selected from the group consisting of —(C₁-C₁₂)alkyl; —(C₂-C₁₂)alkenyl; —(C₂-C₁₂)alkylene-O—(Y—O)_{w}—(C₁-C₁₂)alquil, —(C₂-C₁₂)alkylene-O—(Y—O)_{w}—(C₂-C₁₂)alkylene-NR₁₁R₁₃, —(C₂-C₆)alkynyl; -Cy1; —(C₁-C₁₂)alkylene-Cy1; —(C₂-C₁₂)alkenylene-Cy1; —(C₂-C₁₂)alkynylene-Cy1; -Cy2-(C₁-C₁₂)alkyl; -Cy2-(C₂-C₁₂)alkenyl;
and -Cy2-(C₂-C₁₂)alkynyl; being Cy1 a known ring system independently selected from the group consisting of 5- to 7-membered saturated or partially unsaturated carbocyclic or heterocyclic ring; and 5- to 7-membered carbocyclic or heterocyclic aromatic ring; Cy2 a bivalent known ring system independently selected from the group consisting of 5- to 7-membered saturated or partially unsaturated carbocyclic or heterocyclic ring; and
5- to 7-membered carbocyclic or heterocyclic aromatic ring; and being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl; being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl;
R₅ and R₆ are independently selected from the group consisting of —(C₁-C₁₂)alkyl and an amine protecting group;
R₁₁ and R₁₃ are independently selected from the group consisting of H, —(C₁-C₁₂)alkyl and an amine protecting group;
Halogen is selected from the group consisting of F, Cl, Br, and I;
the detection moiety is selected from the group consisting of chromophore moiety, fluorescent moiety, phosphorescent moiety, luminescent moiety, light absorbing moiety, radioactive moiety, transition metal and isotope mass tag moiety; and
the active moiety is selected from the group consisting of a pharmaceutically active ingredient, a veterinary active ingredient, and a cosmetic active ingredient;
p is an integer from 1 to 10;
q is an integer from 2 to 6;
r is an integer from 2 to 4;
the percentage by weight of the monomer of formula A or a salt thereof is from 30% to 100% by weight;
the percentage by weight of the monomer of formula B or a salt thereof is from 0% to 70% by weight;
being the sum of the percentages equal to 100%; and
the aminosquaramide polymer has a weight average molecular weight (Mw) from 2000 to 91000 Da;
being:
the weight average molecular weight (Mw) from 2000 to 30000 Da measured by Gel Permeation Chromatography-Visible Ultraviolet (GPC-UV); and
the weight average molecular weight (Mw) from higher than 30000 to 91000 Da measured by analytical size-exclusion chromatography coupled with multi-angle light scattering and refractive index detector (SEC/MALS/RI);
an aminosquaramide polymer comprising repeating structural units equal or different of formula (II) or a salt thereof, 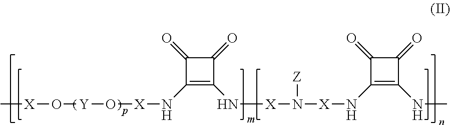
wherein:
m is 0 or 1; and
n is an integer from 10 to 400;
and one polymer as defined in any of the claims 1-22,
wherein at least one of Z and Z′ is a cosmetically active moiety;
or alternatively, a conjugate as defined in any of the claims 23-26 containing it,
or alternatively, a composition as defined in any of the claims 27-29 containing the polymer or the conjugate.

36. A conjugate as defined in any of the claims 23-26; wherein the molecule of interest is at least a molecule selected from the group consisting of a pharmaceutically or veterinary active ingredient, an amino acid-containing compound, a nucleic acid-containing compound, or a mixture thereof, for use in therapy.

37. A conjugate as defined in any of the claims 23-26; wherein the molecule of interest is at least a nucleic acid-containing compound.

38. A composition as defined in any of the claims 27-29 containing it, for use as a transfecting agent.

39. Use in cosmetics of the conjugate as defined in any of the claims 23-26, wherein the molecule of interest is at least one cosmetically active ingredient.

40. A kit comprising:
the polymer selected from the group consisting of aminosquaramide polymer comprising a backbone of repeating structural units, being these repeating structural units equal or different and selected from the group consisting of:
a monomer of formula A, or a salt thereof; 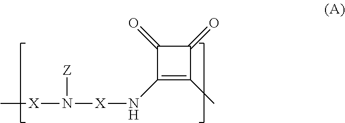
and a monomer of formula B, or a salt thereof; 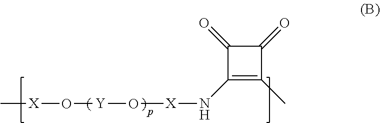
wherein:
X is selected from the group consisting of —(CH₂)_{q}—, —(CHR₁)_{q}— and —(CR₁R₂)_{q}—;
Y is selected from the group consisting of —(CH₂)ᵣ—, —(CHR₃)ᵣ— and —(CR₃R₄)ᵣ—;
R₁ and R₂ are independently selected from the group consisting of H and Z′;
R₃ and R₄ are independently selected from the group consisting of H and Z′;
each Z and Z′ are independently selected from the group consisting of an inert moiety, a detection moiety, and an active moiety;
the inert moiety is selected from the group consisting of —(C₁-C₁₂)alkyl; —(C₂-C₁₂)alkenyl; —(C₂-C₁₂)alkylene-O—(Y—O)_{w}—(C₁-C₁₂)alquil, —(C₂-C₁₂)alkylene-O—(Y—O)—(C₂-C₁₂)alkylene-NR₁₁R₁₃, —(C₂-C₆)alkynyl; -Cy1; —(C₁-C₁₂)alkylene-Cy1; —(C₂-C₁₂)alkenylene-Cy1; —(C₂-C₁₂)alkynylene-Cy1; -Cy2-(C₁-C₁₂)alkyl; -Cy2-(C₂-C₁₂)alkenyl;
and -Cy2-(C₂-C₁₂)alkynyl; being Cy1 a known ring system independently selected from the group consisting of 5- to 7-membered saturated or partially unsaturated carbocyclic or heterocyclic ring; and 5- to 7-membered carbocyclic or heterocyclic aromatic ring; Cy2 a bivalent known ring system independently selected from the group consisting of 5- to 7-membered saturated or partially unsaturated carbocyclic or heterocyclic ring; and
5- to 7-membered carbocyclic or heterocyclic aromatic ring; and being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl; being each one optionally substituted by one or more groups consisting of OH, halogen, NH₂, NHR₅, NR₅R₆, —(C₁-C₆)alkyl, NO₂, N₃, —(C₁-C₁₂)alkyl, —O—(C₁-C₁₂)alkyl, —CO—(C₁-C₁₂)alkyl and —CO—O—(C₁-C₁₂)alkyl;
R₅ and R₆ are independently selected from the group consisting of —(C₁-C₁₂)alkyl and an amine protecting group;
R₁₁ and R₁₃ are independently selected from the group consisting of H, —(C₁-C₁₂)alkyl and an amine protecting group;
Halogen is selected from the group consisting of F, Cl, Br, and I;
the detection moiety is selected from the group consisting of chromophore moiety, fluorescent moiety, phosphorescent moiety, luminescent moiety, light absorbing moiety, radioactive moiety, transition metal and isotope mass tag moiety; and
the active moiety is selected from the group consisting of a pharmaceutically active ingredient, a veterinary active ingredient, and a cosmetic active ingredient;
p is an integer from 1 to 10;
q is an integer from 2 to 6;
r is an integer from 2 to 4;
the percentage by weight of the monomer of formula A or a salt thereof is from 30% to 100% by weight;
the percentage by weight of the monomer of formula B or a salt thereof is from 0% to 70% by weight;
being the sum of the percentages equal to 100%; and
the aminosquaramide polymer has a weight average molecular weight (Mw) from 2000 to 91000 Da;
being:
the weight average molecular weight (Mw) from 2000 to 30000 Da measured by Gel Permeation Chromatography-Visible Ultraviolet (GPC-UV); and
the weight average molecular weight (Mw) from higher than 30000 to 91000 Da measured by analytical size-exclusion chromatography coupled with multi-angle light scattering and refractive index detector (SEC/MALS/RI);
an aminosquaramide polymer comprising repeating structural units equal or different of formula (II) or a salt thereof, 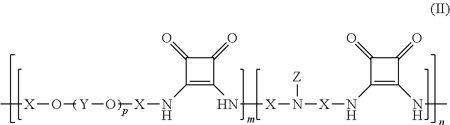
wherein:
m is 0 or 1; and
n is an integer from 10 to 400;
and a polymer as defined in any of the claims 1-22;
or alternatively the composition as defined in any of the claims 27-29 containing the polymer;
optionally means to prepare the conjugate, and
optionally means to administrate the conjugate;
or alternatively, the conjugate as defined in any of the claims 23-26; and optionally means to administrate the conjugate;
or alternatively, the composition as defined in any of the claims 27-29 containing the conjugate, and optionally means to administrate the conjugate.
